(19) United States
(12) Patent Application Publication
(10) Pub. No.: US 2022/0220457 A1
(43) Pub. Date: Jul. 14, 2022

(54) NUCLEIC ACIDS ENCODING IMPROVED TRANSAMINASE PROTEINS

(71) Applicant: BAYER AKTIENGESELLSCHAFT, Leverkusen (DE)

(72) Inventors: Nina BOHLKE, Falkensee (DE); Wayne COCO, Pulheim (DE); Mark James FORD, Niedernhausen (DE); Saskia FUNK, Köln (DE); Ulrike KELLER, Wuppertal (DE); Oliver KENSCH, Pulheim (DE); Ksenia NIESEL, Frankfurt am Main (DE); Nilkolaus PAWLOWSKI, Köln (DE); Moritz SCHON, Köln (DE); Cindy SCHULENBURG, Zürich (CH); Andreas Karl STEIB, Wuppertal (DE); Christina THIES, Dormagen (DE)

(21) Appl. No.: 17/263,885

(22) PCT Filed: Jul. 30, 2019
(86) PCT No.: PCT/EP2019/070431

§ 371 Date: Jan. 27, 2021

(30) Foreign Application Priority Data
Jul. 31, 2018 (EP) 18186729.2

(51) Int. Cl. C12N 9/10; C12N 1/20; C12N 15/70; C12P 13/00; C12P 41/00; C12P 13/04; C12P 13/06; C12P 13/22; C12P 9/00

(52) U.S. Cl. CPC C12N 9/1096; C12N 1/20; C12N 15/70; C12Y 206/01018; C12P 13/001; C12N 2800/101; C12P 13/04; C12P 13/06; C12P 13/22; C12P 9/00; C12P 41/00

(57) ABSTRACT

The present invention concerns proteins having improved omega-transaminase (ω-TA) activity, nucleic acid molecules encoding respective proteins having improved ω-TA activity and methods for stereo selective synthesis of chiral amines and amino acids or increasing of chiral amines isomers in enantiomer mixtures.

### INCORPORATION OF SEQUENCE LISTING

A computer readable form of the Sequence Listing is filed herewith by electronic submission. The Sequence Listing is incorporated by reference in its entirety, is contained in the file created on Sep. 13, 2021, having the file name “BRCS002US-corrected_ST25” and which is 230,391 bytes in size (as measured in MS-Windows operating system).

The present invention concerns proteins having improved omega-transaminase (ω-TA) activity, nucleic acid molecules encoding respective proteins having improved ω-TA activity and methods for stereo selective synthesis of chiral amines and amino acids or increasing of chiral amines isomers in enantiomer mixtures.

Biocatalysis can be based on enzymes available in nature. More often a desire to produce a specific product creates demand for a specific enzyme, which is adapted to economically feasible production of the desired product in large scale. Enzyme engineering is one option for optimizing enzymes towards the economical production of a given product.

Amines and amino acids are ubiquitous in nature not only as parts of proteins and nucleic acids but have also great importance as neurotransmitters (e.g. adrenaline and histamine), as precursor of coenzymes (e.g. cysteamine of coenzyme A) or of complex lipids (e.g. ethanolamine of phosphatidylethanolamine). Especially the higher substituted amines pharmaceutically classified as alkaloids show an enormous variety of structures as well as biological effects found in various forms of life. Biological activities of amines, such as antibiotic, analgesic or neurotoxic activity raises their potential as pharmaceuticals and thus makes them very promising candidates in the search for new drugs. The absolute configuration of the stereocenters of chiral amines is crucial for the interaction with biomolecules and thus for the type of effect on biological systems. For production of a desired target molecule, generation of correct chirality is often a challenge. (Schaetzle, 2011, Inaugural Dissertation, Ernst-Moritz-Arndt-University of Greifswald, Germany, “Identification, characterization and application of novel (R)-selective amine transaminases”).

Many of active compounds in the pipeline of pharmaceutical companies are chiral. Optically active amines belong to important classes of compounds for the synthesis of many active pharmaceutical and agricultural products. L-phenylalanine, e.g. is an important additive in animal feed. No commercially viable process for chemical synthesis of enantiomerically pure amino acids available. Nevertheless, chemical synthesis of racemic amino acids is still of importance, as resolving of the racemic mixtures into pure isomers is possible in some cases by means of biocatalytic processes. (Breuer et al., 2004, Angewandte Chemie International Edition 43, 788-824)

Amine transaminases or ω-transaminases (ω-TAs) are biocatalysts of great importance for the production of chiral primary amines. ω-TAs catalyse the transfer of an amino group from an amino donor onto a carbonyl moiety, utilizing pyridoxal-5′-phosphate (PLP) as cofactor. Thereby the reaction mixture consists of two amines (an amino donor and a product) and two carbonyl compounds (a ketone substrate and a by-product). Both, (S)-selsective and (R)-selective transaminases have been found and well described so far. The enzymes are highly stereo-selective and, thus, have great potential for the direct asymmetric amination, where chiral amines are generated with high enantiomeric excesses directly from an achiral ketone using inexpensive amino donors. (Fesko et al., 2013, J. Molecular Catalysis B, Enzymatic 96, 103-110)

Transaminases have gained attention in biocatalytic synthesis of a wide variety of chiral amines and amino acids. Transaminases can be applied either in the kinetic resolution of racemic amino acids (removing one isomer form a mixture) or in asymmetric synthesis starting from the corresponding pro-chiral keto-substrate. The reaction catalysed by transaminases can be considered a redox reaction with the oxidative deamination of the donor in conjunction with the reductive amination of the acceptor. (Rudat et al., 2012, AMB Express 2:11).

Cann et al. (2012, Org. Process Res. Dev. 16, 1953-1966) disclose the successful use of ω-transaminases for stereoselective production of an α-aminoester, a precursor for production of a migraine headache pharmaceutical. Advantages and disadvantages of the enzymatic versus chemical synthesis are discussed.

U.S. Pat. No. 4,950,606 describes processes for production of optically active amines. In this process ω-transaminases from Bacillus megaterium and Pseudomonas putida convert pro-chiral ketones or keto acids into amines by enantioselective transfer of an amino group from an amino donor. (R)- and (S)-configuration of amines can be obtained.

Park et al. (2013, Organic & Biomolecular Chemistry 11, 6929-6933) discloses the behaviour of different transaminases in enantioselective synthesis of unnatural amino acids from keto acids by using isopropylamine and various other compounds as amine donors.

Park et al. (2013, ChemCatChem 5, 1734-1738) demonstrate the feasibility of using (R)- or (S)-selective ω-transaminases for thermodynamically favourable asymmetric amination of pro-chiral alkyl ketones by using racemic arylalkylamines as an amino donor in a one pot reaction. The reaction does not require addition of excessive amounts of the amino donor or removal of co-products.

Using 2-propylamine, 1-propylamine and racemic-2-butylamine as amino donor ω-transaminase catalysed reactions have been demonstrated to lead to up to three times higher conversions compared to reactions wherein alanine was used as amino donor. The amino acids β-alanine and asparagine were poor amino donors. For some methyl ketones containing an aromatic residue, the optically pure amines were obtained with high yields when an excess of 2-butylamine or 1-phenylethylamine was used as amino donor. No further steps for shifting the equilibrium were required. (Fesko et al., 2013, J. Molecular Catalysis B, Enzymatic 96, 103-110)

Shin & Kim (2001, Biosci. Biotechno. Biochem. 65(8), 1782-1788) disclose the isolation of ω-transaminases using aryl amines including (S)-α-methylbenzylamine ((S)-α-MBA), 1-methyl-3-phenylpropyl-amine, 1-aminotetralin or 1-aminoindan as amine donor. Good amino acceptors were found to be the ketoacids pyruvate and glyoxylate or the aldehydes propionaldehyde and butaraldehyde.

U.S. Pat. No. 6,133,018 discloses the production of (S)-1-methoxy-2-aminopropane by bringing methoxyacetone and the achiral amino donor 2-aminopropane into contact with ω-transaminase.

A four enzyme system for production of D-amino acids by conversion of a keto acid into the respective D-amino acid catalysed by a D-amino acid aminotransferase (transaminase) using D-alanine as an amino donor is described in Galkin et al. (1997, J. Fermentation and Bioengeneering 83(3), 299-300) as. For driving the reaction equilibrium in direction of D-amino acids further reactions were coupled to the D-amino acid aminotransferase. Pyruvate and ammonia are converted into L-alanine by alanine dehydrogenase simultaneously reducing NADH to NAD. L-alanine is converted by alanine racemase into D-alanine. Recycling of NADH from NAD is established by formation of carbon dioxide from formic acid catalysed by formate dehydrogenase. Pyruvate is recycled from alanine by the D-amino acid aminotransferase reaction. D-enantiomers of glutamate, leucine, norleucine and methionine could be produced in high yield, whereas D-phenylalanine and D-tyrosine were synthesized at low yields, D-Norvaline could only be produced in access of 30% and aminobutyrate was produced only as a racemic mixture.

WO 2010/089171 A2 discloses methods for ammonizing at least one keto group in an at least one keto group comprising multi-cyclical ring system into an amino group in reactions catalysed by enzymes having transaminase activity.

WO 2015/195707 A1 (US2015361468 A1) discloses the production of five carbon polymer building blocks by transgenic bacteria. Bacterial biosynthetic pathways are manipulated by introduction of multiple enzymes including ω-transaminases. ω-transaminases are demonstrated to catalyse reactions of glutarate semi-aldehyde to 5-aminopentanoate and the reverse reaction, 5-aminopentanol to 5-oxopentanol, cadaverine to 5-aminopentanal, N5-acetyl-1,5-diaminopentane to N5-acetyl-5-aminopentanal. L-glutamate/2-oxoglutarate or L-alanine/pyruvate were used as amino donor/acceptor, respectively.

KR 20030072067 discloses isolation of a thermophyllic Bacillus sp. T30 strain comprising an L-selective aromatic amino acid transferase (transaminase) and the use of this strain as a biocatalyst for the production of aromatic L-amino acids at high reaction temperatures thereby increasing solubility of the keto acid substrate.

Koszelewski et al. (2010, ChemCat Chem 2(1), 73-77, including “Supporting Information”) discloses the use of whole cell catalysts for the synthesis of enantiomerically pure amines from corresponding pro-chiral amines and the resolution of racemic amines. Different ω-transaminases from Bacillus megaterium SC6394, Alcaligenes denitrificans Y2k-2, Chromobacterium violaceum DSM30191, the W57G mutant of the ω-transaminase of Vibrio fluvialis and a mutant termed CNB05-01, originating from an Arthrobacter species are expressed in Escherichia coli cells. Lyophylized Escherichia coli cells were used for kinetic resolution and stereoselective amination reactions.

The product range accessible by the use of transaminases is limited by the characteristics of most natural occurring ω-transaminases to not accept substrates bulkier than an ethyl group at a position adjacent to the ketone (Savile et al., 2010, Science 329, 305-309, including “Supporting Information”). Park et al. (2014, Adv. Synth.Catal. 356, 212-220) discovered an (S)-selective ω-transaminase from Paracoccus denitrificans which does accept substrates with substituents up to an n-butyl group (i.e. 2-oxohexanoate n-hexyl) but did not accept branched chain α-keto acids. A variant (V153A) of the (S)-selective ω-transaminase from Paracoccus denitrificans did show improved activity towards the linear keto acid (S)-1-phenylbutylamine but did not accept branched keto acids.

Variants of a mesophilic Arthrobacter citreus ω-transaminase comprising seventeen amino acid substitutions compared to the amino acid sequence of the respective wild-type sequence show improved thermostability and significantly improved specific activity in reactions producing substituted (S)-aminotetralin from substituted tetralone in presence of isopropylamine as amine donor. (Martin et al., 2007, Biochemical Engineering Journal 37, 246-255)

Savile et al. (2010, Science 329, 305-309, including “Supporting Information”) disclose the manufacture of the complex antidiabetic pharmaceutical sitagliptin by a biocatalytic process involving an ω-transaminase. Various variants of an Arthrobacter sp. (R)-selective ω-transaminase (ATA-117) were produced. The enzymes show a broad substrate range, increased tolerance to isopropylamine and organic solvents. Various trifluoromethhyl-substituted amines and phenylamines could be produced by these enzymes. An optimized variant of an Arthrobacter sp. (R)-selective ω-transaminase (ATA-117) containing 27 amino acid substitutions compared to the wild-type enzyme was used to produce sitagliptin by amination of prositagliptin ketone in presence of isopropylamine as amine donor.

WO 2006/06339 (U.S. Pat. No. 7,247,460) discloses Arthrobacter citerus ω-transaminase variants which are thermostable, have an increased reaction rate and tolerance to high amine donor concentrations, in each case when compared to the respective wild-type enzyme.

Although several improvements of transaminases have been achieved so far, limitations arising during the asymmetric synthesis of amines or resolution of racemic amines, such as unfavourable equilibrium, substrate and product inhibition, poor thermostability, insufficient substrate specificity and sometimes low enantioselectivity of the transaminase, still have to be overcome for an efficient production of a wide range of amines on industrial scale.

Thus, there is a need for further improvement of ω-transaminases. In particular in respect to production of desired aminated, enantiomerically enriched or pure products, preferably under specific and/or economically viable production processes improved further ω-transaminases are needed.

The present invention provides ω-transaminases (ω-TAs) variants comprising modifications in their amino acid sequence or further modified variants of ω-TAs comprising additional modifications in their amino acid sequence, these variants and further modified variants comprising further amino acid modifications having improved reaction kinetics, improved substrate acceptance and improved specific activity in comparison to respective wild-type ω-TAs. The variants and variants comprising further amino acid modifications of the invention therefore enable the development of economically efficient production processes for aminated products in production methods of new aminated products or precursors of respective products not achievable by the use of the respective wild-type ω-TAs.

The variants or further modified variants of ω-TAs described herein have advantages over known wild-type and other already known ω-TAs. In particular the modified or variant ω-TAs described herein have the advantage that they can produce enantiomerically enriched or enantiomerically nearly pure or pure compounds, like e.g. branched or aromatic amino acids which cannot be produced with respective wild-type ω-transaminases. The further modified variants of ω-TAs described herein have the advantage that they can produce enantiomerically enriched, nearly pure or pure compounds of phospho-amino acids.

Positions 1 to 477 in SEQ ID NO 3 represent the amino acid sequence of a wild-type ω-transaminase (ω-TA) from Bacillus megaterium derivable from GenPept (PDB) under accession No 5G09_A.

Positions 1 to 479 in SEQ ID NO 6 represent the amino acid sequence of a wild-type ω-TA from Arthrobacter sp. derivable from GenPept (PDB) under accession No 5G2P_A.

Positions 1 to 476 in SEQ ID NO 9 represent the amino acid sequence of a wild-type ω-TA from Bacillus sp. (soil 76801 D1 derivable from GenPept (PDB) under accession No. KRF52528.1.

Positions 1 to 476 in SEQ ID NO 12 represent the amino acid sequence of an ω-TA variant from Arthrobacter sp. derivable from SEQ ID NO 16 in WO 2006/06336 A2.

Positions 1 to 476 in SEQ ID NO 15 represent the amino acid sequence of a wild-type ω-TA from Arthrobacter sp. derivable from SEQ ID NO 2 in WO 2006/06336 A2.

Described herein are proteins having the activity of an ω-TA, wherein the amino acid sequences of these proteins represent variants of known proteins having the activity of an ω-TA. In particular, the amino acid sequence of proteins having the activity of an ω-TA described herein represent variants of the amino acid sequences represented by amino acids from positions 1 to 477 in SEQ ID NO 3 and/or represented by amino acids from positions 1 to 479 in SEQ ID NO 6 and/or represented by amino acids from positions 1 to 476 in SEQ ID NO 9 and/or represented by amino acids from positions 1 to 476 in SEQ ID NO 12 and/or represented by amino acids from positions 1 to 476 in SEQ ID NO 15, wherein in each of the amino acid sequences shown under SEQ ID NO 3, SEQ ID NO 6, SEQ ID NO 9, SEQ ID NO 12 and SEQ ID NO 15 at least 5 the amino acids at positions 25, 64, 88, 157, 165, 169, 174, 187, 197, 239, 327, 328, 384, 389, 391, 396, 410 and 414 are different from those amino acids given at the respective amino acid position in each of the sequences shown under SEQ ID NO 3, SEQ ID NO 6, SEQ ID NO 9, SEQ ID NO 12 and SEQ ID NO 15, respectively.

The abbreviation “ω-TA” is used and meaning herein “ω-transaminase”.

The term “variant” as used herein means subject-matter which is different from subject-matter known in the art. In respect with nucleic acid molecules and proteins variants are understood to comprise a nucleic acid sequence or an amino acid sequence, respectively, which deviates from accordingly known sequences but encode a protein having the same function or catalysing the same reaction e.g. the function of encoding a protein having the activity of an ω-TA. Deviation of nucleic acid molecule sequences and amino acid sequences from known nucleic acid sequences and protein sequences means that the sequences comprise substitutions (replacements) and/or deletions and/or insertions of nucleotides or amino acids, respectively, in comparison to the correspondingly known nucleic acid sequences or amino acid sequences.

A first embodiment of the invention concerns proteins having the activity of an ω-TA, wherein the proteins are selected from the group consisting of
a) proteins comprising the amino acid sequence from positions 1 to 477 as shown under SEQ ID NO 3 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
b) proteins comprising the amino acid sequence from positions 1 to 479 as shown under SEQ ID NO 6 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from T and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
c) proteins comprising the amino acid sequence from positions 1 to 476 as shown under SEQ ID NO 9 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
d) proteins comprising the amino acid sequence from positions 1 to 476 as shown under SEQ ID NO 12 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 and the amino acid at position 187 is different from S is different from E and the amino acid at position 197 is different from T the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
e) proteins comprising the amino acid sequence from positions 1 to 476 as shown under SEQ ID NO 15 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 and the amino acid at position 187 is different from S is different from E and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
f) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequence's shown under a), b), c), d), e) or f), given that in each case the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid corresponding to position 197 is different from T or M and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P.

The meaning of amino acid abbreviations A, C, D, E, F, G, H, I, K, L, M, N, P, Q, R, S, T V, W, Y is derivable herein below Table 4 under the paragraph sub-titled “Description of the Sequences”.

An “amino acid corresponding to position x” in a first amino acid sequence (e.g. positions 64 in SEQ ID NO 3) means herein that an amino acid of a second amino acid sequence when compared with a first amino acid sequence appears at position x of the first amino acid sequence in a pairwise sequence alignment of the first amino acid sequence with the second amino acid sequence in case the numbering of the amino acids of the second amino acid sequence differs from the amino acid numbering of the first amino acid sequence.

In the context of the present invention, the term “identity” in respect to sequence identity or sequences being identical to is to be understood as meaning the number of identical amino acids or nucleotides shared over the entire sequence length by a first nucleic or amino acid sequence with another (second) nucleic or amino acid sequence, respectively, expressed in percent.

“Sequence identity” can be determined by alignment of two amino acid or two nucleotide sequences using global or local alignment algorithms comprised for example in known software like GAP or BESTFIT or the Emboss program “Needle”. These software use the Needleman and Wunsch global alignment algorithm for aligning two sequences, over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using software, such as EMBOSS, accessible at world wide web site of the EBI (ebi.ac.uk/Tools/emboss/). Alternatively sequence similarity or identity may be determined by searching against databases (e.g. EMBL, GenBank) by using commonly known algorithms and output formats such as FASTA, BLAST, etc., but preferably hits should be retrieved and aligned pairwise to finally determine sequence identity.

Preferably, identity with respect to a protein having the activity of an ω-TA is determined by comparisons with the amino acid sequence given under SEQ ID NO 18 and the identity with respect to a nucleic acid molecule coding for a protein having the activity of an ω-TA is determined by comparisons of the nucleic acid sequence given under SEQ ID NOs 16 or 17 with other proteins or nucleic acid molecules, respectively, with the aid of computer programs. If sequences to be compared with one another are of different length, the identity is to be determined by determining the identity in percent of the number of amino acids or nucleotides, respectively, which the shorter sequence shares with the longer sequence. Preferably, the identity is determined using the known and publicly available computer program ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from various Internet pages, inter alia from IGBMC (Institut de Génétique et de Biologie Moléculaire et Cellulaire, B.P.163, 67404 Illkirch Cedex, France; ftp://ftp-igbmc.u-strasbg.fr/pub/) and from EBI (ftp://ftp.ebi.ac.uk/pub/software/) and all mirrored Internet pages of the EBI (European Bioinformatics Institute, Wellcome Trust Genome Campus, Hinxton, Cambridge CB10 1SD, UK).

Preferably, use is made of the ClustalW computer program of version 1.8 to determine the identity between proteins described in the context of the present invention and other proteins. Here, the parameters have to be set as follows: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, use is made of the ClustalW computer program of version 1.8 to determine the identity for example between the nucleotide sequence of the nucleic acid molecules described in the context of the present invention and the nucleotide sequence of other nucleic acid molecules. Here, the parameters have to be set as follows: KTUPLE=2, TOPDIAGS=4, PAIRGAP=5, DNAMATRIX:IUB, GAPOPEN=10, GAPEXT=5, MAXDIV=40, TRANSITIONS: unweighted.

Identity furthermore means that there is a functional and/or structural equivalence between the nucleic acid molecules in question or the proteins encoded by them. Functional equivalence means that the nucleic acid molecule sequences or the amino acid sequences encode a protein having the activity of an ω-TA. The nucleic acid molecules which are homologous to the molecules described above and represent derivatives of these molecules are generally variants of these molecules which represent modifications having the same biological function or catalysing the same reaction, i.e. coding for a protein having the activity of an ω-TA. They may be either naturally occurring variants, for example sequences from other species, or mutations, where these mutations may have occurred in a natural manner or were introduced by targeted mutagenesis. Furthermore, the variants may be synthetically produced sequences. The allelic variants may be either naturally occurring variants or synthetically produced variants or variants generated by recombinant DNA techniques. However, concerning the present invention it is decisive that those variants encode proteins having ω-TA activity and comprise the amino acid substitutions (replacements), deletions or insertions described herein concerning the proteins according to the invention.

A special type of derivatives are, for example, nucleic acid molecules which differ from the nucleic acid molecules described in the context of the present invention as a result of the degeneracy of the genetic code.

According to the NC-IUBMB (Nomenclature Committee of the International Union of Biochemistry and Molecular Biology) transaminases (TAs) belong to the class of transferases (EC 2). Transferases are enzymes transferring a group, e.g. a methyl group or a glycosyl group, from one compound (generally regarded as donor) to another compound (generally regarded as acceptor). The group of transferases comprises enzymes transferring nitrogenous groups (EC 2.6). The reaction catalysed by TAs can be formally considered a redox reaction with the oxidative deamination of a(n) (amine) donor in conjunction with the reductive amination of a carbonyl acceptor by transferring a —NH₂ group and —H to a compound containing a carbonyl group in exchange for the ═O of that group according to the general equation (I)

R¹—CH(—NH₂)—R²+R³—CO—R⁴→R¹—CO—R²+R³—CH(—NH₂)—R⁴.

The reverse reaction which is also catalysed by TAs can be formally described according to the general equation (Ia)

R¹—CO—R²+R³—CH(—NH₂)—R⁴→R¹—CH(—NH₂)—R²+R³—CO—R⁴.

TAs are pyridoxal 5′-phosphate (PLP)-dependent enzymes. The unique distinctive feature of TA catalysed reactions is the transfer of an amino group (by a well-established mechanism involving covalent substrate-coenzyme intermediates), which justifies allocation of these enzymes among the transferases into a special subclass, designated transaminases or amino transferases (EC 2.6.1).

TAs are commonly further classified in the art as α-TAs and ω-TAs. This nomenclature is based on the relative position of the amino group of amino acids which is transferred by respective TAs. In respect to amine carboxylic acids α-TAs catalyse transamination of amino groups of an α-carbon only, wherein ω-TAs also act on non-α-amines and transfer the distal amino group of the respective substrate. (Shin et al., 2003, Appl Microbiol Biotechnol 61, 463-471). However, it is known in the art that some ω-TAs are able to catalyse transamination of (primary) amine compounds not bearing a carboxyl group (Rudat et al., 2012, AMB Express 2(11); Shin et al., 2003, Appl Microbiol Biotechnol 61, 463-471).

If a protein has the activity of a TA, in particular an ω-TAs can be detected with methods known and described in the art. An assay for detecting ω-TA activity of proteins based on blue staining of α-amino acids with a CuSO₄/MeOH was developed by Hwang & Kim (2004, Enzyme and Microbiol Technology 34(5), 429-436). Truppo et al. (2009, Org. Biomol. Chem. 7, 395-398) describe an assay for high throughput screening for ω-TAs based on a multi-enzyme cascade pH-indicator assay and in addition discloses a conventional HPLC analysis assay.

It is not decisive which method is used for detecting if a protein according to the invention has the activity of an ω-TA. Preferably, in connection with the present invention, the method described under “General Methods”, item 4 is used for detecting if a protein according to the invention has the activity of an ω-TA, in particular this method is used for detecting if a an ω-TA variant according to the invention has the activity of an ω-TA.

Concerning an ω-TA variants comprising further amino acid modifications, preferably the method described under “General Methods”, item 7 is used for detecting if a protein according to the invention has the activity of an ω-TA

In a preferred embodiment of the invention the protein according to the invention is an (S)-selective ω-TA.

The term “(S)-selective” means in connection with the present invention that reductive amination of the (amine) acceptor according to general equation (I) produces the (S)-enantiomer in enantiomeric excess over the (R)-enantiomer.

The reaction catalysed by (S)-selective ω-TAs can be formally described according to the general equation (II)

R¹—CH(—NH₂)—R²+R³—CO—R⁴→R¹—CO—R²+R³—CH((S)—NH₂)—R⁴.

ω-TA variant proteins according to the invention may exhibit additional amino acid modifications (amino acid substitutions, deletions or insertions) compared to the amino acid sequences described herein above in respect to the amino acid sequences shown under SEQ ID Nos 3, 6, 9, 12 or 15.

In addition to the ω-TA variants described herein above under item a) or c) the amino acid sequence shown from position 1 to 477 under SEQ ID NO 3 or the amino acid sequence shown from position 1 to 477 under SEQ ID NO 9, respectively, can have additional amino acid substitutions at positions 2 and/or 48 and/or 164 and/or 242 and/or 245 and/or 311 and/or 353 and/or 424 and/or the amino acid sequence shown under SEQ ID NO 3 can have additional amino acid substitutions at positions 202 and/or 205 and/or 359 and/or 475 and/or 476 and/or a deletion of the amino acid at position 477 and/or the amino acid sequence shown under SEQ ID NO 9 can have additional amino acid substitutions at positions 69 and/or 90 and/or 268 and/or 318 and/or 322 and/or 452.

In addition to the ω-TA variants described above under items b) and d) each of the amino acid sequences shown from positions 1 to 479 under SEQ ID NO 6 or the amino acid sequences shown from positions 1 to 476 under SEQ ID NO 12, respectively, can have additional amino acid substitutions at positions 46 and/or 60 and/or 185 and/or 186 and/or 195 and/or 205 and/or 252 and/or 268 and/or 409 and/or 436 and/or in the amino acid sequence shown under SEQ ID NO 6 the amino acids at positions 477 and/or 478 and/or 479 can be deleted.

In addition to the ω-TA variants described above under item e) the amino acid sequence shown from positions 1 to 476 under SEQ ID NO 15 can have additional amino acid substitutions at positions 48 and/or 164 and/or 242 and/or 245 and/or 255 and/or 424.

A further embodiment of the invention therefore concerns proteins according to the invention comprising additional amino acid modifications, preferably those embodiments are proteins having the activity of an ω-TA, wherein the proteins are selected from the group consisting of
a) proteins comprising the amino acid sequence from positions 1 to 477 as shown under SEQ ID NO 3 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P and the amino acid at position 2 is different from S and the amino acid at position 48 is different from D and the amino acid at position 164 is different from Y and the amino acid at position 202 is different from D and the amino acid at position 205 is different from L and the amino acid at position 242 is different from A and the amino acid at position 245 is different from A and the amino acid at position 311 is different from L and the amino acid at position 353 is different from F and the amino acid at position 359 is different from D and the amino acid at position 424 is different from K and the amino acid at position 475 is different from A and the amino acid at position 476 is different from L and the amino acid at position 477 is deleted;
b) proteins comprising the amino acid sequence from positions 1 to 479 as shown under SEQ ID NO 6 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from T and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P and the amino acid at position 46 is different from T and the amino acid at position 60 is different from C and the amino acid at position 185 is different from C and the amino acid at position 186 is different from S and the amino acid at position 195 is different from S and the amino acid at position 205 is different from Y and the amino acid at position 252 is different from V and the amino acid at position 268 is different from S and the amino acid at position 409 is different from R and the amino acid at position 436 is different from A and the amino acids at positions 477 and 478 and 479 are deleted;
c) proteins comprising the amino acid sequence from positions 1 to 476 as shown under SEQ ID NO 9 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P and the amino acid at position 2 is different from S and the amino acid at position 48 is different from D and the amino acid at position 69 is different from P and the amino acid at position 90 is different from S and the amino acid at position 164 is different from Y and the amino acid at position 242 is different from A and the amino acid at position 245 is different from A and the amino acid at position 268 is different from T and the amino acid at position 311 is different from L and the amino acid at position 318 is different from E and the amino acid at position 322 is different from R and the amino acid at position 353 is different from S and the amino acid at position 424 is different from K and the amino acid at position 452 is different from E;
d) proteins comprising the amino acid sequence from positions 1 to 476 as shown under SEQ ID NO 12 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from T the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P and the amino acid at position 46 is different from T and the amino acid at position 60 is different from C and the amino acid at position 185 is different from C and the amino acid at position 186 is different from C and the amino acid at position 195 is different from S and the amino acid at position 205 is different from Y and the amino acid at position 252 is different from V and the amino acid at position 268 is different from S and the amino acid at position 409 is different from R and the amino acid at position 436 is different from A;
e) proteins comprising the amino acid sequence from positions 1 to 476 as shown under SEQ ID NO 15 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P and the amino acid at position 48 is different from D and the amino acid at position 164 is different from Y and the amino acid at position 242 is different from A and the amino acid at position 245 is different from A and the amino acid at position 255 is different from F and the amino acid at position 424 is different from K;
f) proteins having an amino acid sequence being at least 60%, preferably 70%, more preferably 80%, further more preferably 90%, even more preferably 95%, even further more preferably 96%, particular preferably 97%, most preferably 98% or especially preferably 99% identical to any of the amino acid sequences as defined under a) (amino acid sequence from positions 1 to 477 as shown under SEQ ID NO 3) given that the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid at position 197 is different from M and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P and the amino acid corresponding to position 2 is different from S and the amino acid corresponding to position 48 is different from D and the amino acid corresponding to position 164 is different from Y and the amino acid corresponding to position 202 is different from D and the amino acid corresponding to position 205 is different from L and the amino acid corresponding to position 242 is different from A and the amino acid corresponding to position 245 is different from A and the amino acid corresponding to position 311 is different from L and the amino acid corresponding to position 353 is different from F and the amino acid corresponding to position 359 is different from D and the amino acid corresponding to position 424 is different from K and the amino acid corresponding to position 475 is different from A and the amino acid corresponding to position 476 is different from L and the amino acid corresponding to position 477 is deleted;
g) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequences as defined under b) (positions 1 to 476 as shown under SEQ ID NO 6) given that the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid at position 197 is different from T and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P and the amino acid at position 46 is different from T and the amino acid corresponding to position 60 is different from C and the amino acid corresponding to position185 is different from C and the amino acid corresponding to position 186 is different from S and the amino acid corresponding to position 195 is different from S and the amino acid corresponding to position 205 is different from Y and the amino acid corresponding to position 252 is different from V and the amino acid corresponding to position 268 is different from S and the amino acid corresponding to position 409 is different from R and the amino acid corresponding to position 436 is different from A and the amino acids corresponding to positions 477 and 478 and 479 are deleted;
h) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequences as defined under c) (positions 1 to 479 as shown under SEQ ID NO 9) given that the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid at position 197 is different from M and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P and the amino acid corresponding to position 2 is different from S and the amino acid corresponding to position 48 is different from D and the amino acid corresponding to position 69 is different from P and the amino acid corresponding to position 90 is different from S and the amino acid corresponding to position 164 is different from Y and the amino acid corresponding to position 242 is different from A and the amino acid corresponding to position 245 is different from A and the amino acid corresponding to position 268 is different from T and the amino acid corresponding to position 311 is different from L and the amino acid corresponding to position 318 is different from E and the amino acid corresponding to position 322 is different from R and the amino acid corresponding to position 353 is different from S and the amino acid corresponding to position 424 is different from K and the amino acid corresponding to position 452 is different from E;
i) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequences as defined under d) (positions 1 to 476 as shown under SEQ ID NO 12 given that the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid at position 197 is different from T and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P and the amino acid corresponding to position 46 is different from T and the amino acid corresponding to position 60 is different from C and the amino acid corresponding to position 185 is different from C and the amino acid corresponding to position 186 is different from C and the amino acid corresponding to position 195 is different from S and the amino acid a corresponding to position 205 is different from Y and the amino acid corresponding to position 252 is different from V and the amino acid corresponding to position 268 is different from S and the amino acid corresponding to position 409 is different from R and the amino acid corresponding to position 436 is different from A;
j) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequences as defined under e) (positions 1 to 476 as shown under SEQ ID NO 15) given that the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid at position 187 is different from S and the amino acid at position 197 is different from M and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P and the amino acid at position 48 is different from D and the amino acid at position 164 is different from Y and the amino acid at position 242 is different from A and the amino acid at position 245 is different from A and the amino acid at position 255 is different from F and the amino acid at position 424 is different from K.

Positions 1 to 476 in SEQ ID NO 18 represent the amino acid sequence of an ω-TA variant protein comprising all the amino acid modifications described herein above in comparison to each of the amino acid sequences as shown under SEQ ID NO 3 (from positions 1 to 477), SEQ ID NO 6 (from positions 1 to 479), SEQ ID NO 9 (from positions 1 to 476), SEQ ID NO 12 (from positions 1 to 476) and SEQ ID NO 15 (from positions 1 to 476).

Table 1 summarizes the modifications present in the amino acid sequence of an ω-TA variant protein according to the invention (positions 1 to 476 under SEQ ID NO 18) in comparison to each of the amino acid sequences of wild-type ω-TAs (positions 1 to 477 under SEQ ID NO 3 or positions 1 to 479 under SEQ ID NO 6 or positions 1 to 476 under SEQ ID NO 9 or positions 1 to 476 under SEQ ID NO 15) as well as in comparison to a modified ω-TA from Arthrobacter sp. (positions 1 to 476 under SEQ ID NO 12).

TABLE 1
“End” in Table 1 indicates the position after the last amino
acid present in the amino acid sequence of the respectively
known (wild-type) sequence.

| | Amino acid present at respective amino acid | | | | | |
|---|---|---|---|---|---|---|
| Amino | position in | | | | | |
| acid | SEQ ID | SEQ ID | SEQ ID | SEQ ID | SEQ ID | SEQ ID |
| position | NO 3 | NO 6 | NO 9 | NO 12 | NO 15 | NO 18 |
| 2 | S | G | S | G | G | G |
| 25 | F | F | F | F | F | R |
| 46 | M | T | M | T | M | M |
| 48 | D | G | D | G | D | G |
| 60 | Y | C | Y | C | Y | Y |
| 64 | L | L | L | L | L | I |
| 69 | Q | Q | P | Q | Q | Q |
| 88 | T | T | T | T | T | A |
| 90 | A | A | S | A | A | A |
| 157 | T | T | T | T | T | A |
| 164 | Y | F | Y | F | Y | F |
| 165 | R | R | R | R | R | Q |
| 169 | V | V | V | V | V | A |
| 174 | E | E | E | E | E | G |
| 185 | Y | C | Y | C | Y | Y |
| 186 | N | S | N | S | N | N |
| 187 | S | S | S | S | S | N |
| 195 | P | S | P | S | P | P |
| 197 | M | T | M | T | M | A |
| 202 | D | N | N | N | N | N |
| 205 | L | Y | C | Y | C | C |
| 239 | S | S | S | S | S | P |
| 242 | A | V | A | V | A | V |
| 245 | A | T | A | T | A | T |
| 252 | I | V | I | V | I | I |
| 255 | I | I | I | I | F | I |
| 268 | N | S | T | S | N | N |
| 311 | L | V | L | V | V | V |
| 318 | A | A | E | A | A | A |
| 322 | K | K | R | K | K | K |
| 327 | S | S | S | S | S | T |
| 328 | V | V | V | V | V | G |
| 353 | F | L | F | L | L | L |
| 359 | D | N | N | N | N | N |
| 384 | Y | Y | Y | Y | Y | C |
| 389 | I | I | I | I | I | L |
| 391 | D | D | D | D | D | E |
| 396 | K | K | K | K | K | E |
| 409 | T | R | T | R | T | T |
| 410 | H | H | H | H | H | R |
| 414 | P | P | P | P | P | L |
| 424 | K | E | K | E | K | E |
| 436 | V | A | V | A | V | V |
| 452 | G | G | E | G | G | G |
| 475 | A | Q | Q | Q | Q | Q |
| 476 | L | S | S | S | S | S |
| 477 | E | A | End | End | End | End |
| 478 | End | L | | | | |
| 479 | | E | | | | |
| 480 | | End | | | | |

A preferred embodiment of the invention therefore concerns a protein according to the invention having the activity of an ω-TA selected from the group consisting of
a) proteins comprising the amino acid sequence from positions 1 to 476 as shown under SEQ ID NO 18;
b) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with the amino acid sequence from positions 1 to 476 as shown under SEQ ID NO 18 given that each of the amino acids corresponding to positions 25, 64, 88, 157, 165, 169, 174, 187, 197 239, 327, 328, 384, 389, 391, 396, 410 and 414 in SEQ ID NO 18 represent those amino acids shown at the respective positions in the amino acid sequence shown under SEQ ID NO 18;
c) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with the amino acid sequence from positions 1 to 476 as shown under SEQ ID NO 18 given that each of the amino acids corresponding to positions 2, 25, 46, 48, 60, 64, 69, 88, 90, 157, 164, 165, 169, 174, 185, 186, 187, 195, 197, 202, 205, 239, 242, 245, 252, 255, 268, 311, 318, 322, 327, 328, 353, 359, 384, 389, 391, 396, 409, 410, 414, 424, 436, 452, 475 and 476 in SEQ ID NO 18 represent those amino acids shown at the respective positions in the amino acid sequence shown under SEQ ID NO 18.

In the most preferred embodiment the protein according to the invention encoding an ω-TA is a protein comprising the amino acid sequence from positions 1 to 476 as shown under SEQ ID NO 18.

The proteins so far described herein above are commonly referred to herein as ω-TA variants or protein variants according to the invention.

It was found that introduction of further amino acid modifications into protein variants according to the invention further improves the activity of the ω-TA variants, in particular in respect to its substrate specificity, meaning that these further modified ω-TA variants are better adapted to produce enantiomerically enriched or nearly pure products compared to the ω-TA variants described herein above. The ω-TAs comprising further modifications are further modified compared to the ω-TA variants described herein above as proteins according to the invention. The ω-TA variants comprising further modifications are in particular suitable for producing enantiomerically enriched or enantiomerically nearly pure phospho-amino acids and are designated herein ω-TA variants comprising further amino acid modifications or proteins according to the invention comprising further amino acid modifications.

In respect to ω-TA variants having further amino acid modifications preferred methods for showing that a protein has the activity of an ω-TA are e.g. described in WO 2017/151573, a particular preferred method for demonstrating that ω-TA variants having further amino acid modifications is described herein under “General Methods”, item 7.

“Enantiomerically enriched” means herein that one of two enantiomers is present in a composition in higher amounts than the other enantiomer, preferably at least 60% of one enantiomer is present in the composition, more preferably at least 65% of one enantiomer is present in the composition, further more preferably at least 70% of one enantiomer is present in the composition, even more preferably at least 75% of one enantiomer is present in the composition, even further more preferably at least 80% of one enantiomer is present in the composition, particular preferably at least 85% of one enantiomer is present in the composition, most preferably at least 90% of one enantiomer is present in the composition or especially preferably at least 94% of one enantiomer is present in the composition.

“Enantiomerically nearly pure means herein that one of two enantiomers is present in a composition in amounts of at least 95.0%, preferably one of two enantiomers is present in a composition in amounts of at least 95.5%, more preferably one of two enantiomers is present in a composition in amounts of at least 96.0%, further more preferably one of two enantiomers is present in a composition in amounts of at least 96.5%, even more preferably one of two enantiomers is present in a composition in amounts of at least 97.0%, even further more preferably one of two enantiomers is present in a composition in amounts of at least 98.0%, particular preferably one of two enantiomers is present in a composition in amounts of at least 98.5%, most preferably one of two enantiomers is present in a composition in amounts of at least 99.0%, or especially preferably one of two enantiomers is present in a composition in amounts of at least 99.5%.

Another embodiment according to the invention therefore concerns protein variants according to the invention having the activity of an ω-TA variant, wherein the amino acid sequences according to the invention comprise further amino acid modifications in comparison to the proteins according to the invention.

Preferably another embodiment of the invention in respect to amino acid sequences of proteins having the activity of an ω-TA according to the invention (ω-TA variants) comprising further amino acid modifications therefore is a protein according to the invention having the activity of an ω-TA selected from the group consisting of
a) proteins according to the invention given that the amino acid at position 166 is G and the amino acid at position 327 is Q;
b) proteins according to the invention given that the amino acid at position 327 is Q and the amino acid at position 384 is S;
c) proteins according to the invention given that the amino acid at position 326 is Q and the amino acid at position 327 is Q;
d) proteins according to the invention given that the amino acid at position 327 is Q;
e) proteins according to the invention given that the amino acid at position 326 is F and the amino acid at position 327 is Q;
f) proteins according to the invention given that the amino acid at position 327 is C;
g) proteins according to the invention given that the amino acid at position 327 is I;
h) proteins according to the invention given that the amino acid at position 327 is M;
i) proteins according to the invention given that the amino acid at position 164 is Y;
j) proteins according to the invention given that the amino acid at position 164 is S;
k) proteins according to the invention given that the amino acid at position 327 is V;
l) proteins according to the invention given that the amino acid at position 409 is R;
m) proteins according to the invention given that the amino acid at position 327 is S;
n) proteins according to the invention given that the amino acid at position 271 is I;
o) proteins according to the invention given that the amino acid at 329 is G;
p) proteins according to the invention given that the amino acid at position 409 is P;
q) proteins according to the invention given that the amino acid at position 414 is M;
r) proteins according to the invention given that the amino acid at position 165 is K;
s) proteins according to the invention given that the amino acid at position 414 is R;
t) proteins according to the invention given that the amino acid at position 414 is H;
u) proteins according to the invention given that the amino acid at position 165 is C;
v) proteins according to the invention given that the amino acid at position 327 is V;
w) proteins according to the invention given that the amino acid at position 164 is C;
x) proteins according to the invention given that the amino acid at position 409 is K.

A more preferred embodiment of the invention in respect to amino acid sequences of proteins having the activity of an ω-TA comprising further amino acid modifications concerns proteins having the activity of an ω-TA selected from the group consisting of
a) proteins having the amino acid sequence from positions 1 to 476 in the amino acid sequence shown under SEQ ID NO 18, apart from that the amino acid S at position 166 in SEQ ID NO 18 is substituted by G and the amino acid Tat position 327 in SEQ ID NO 18 is substituted by Q;
b) proteins having the amino acid sequence from positions 1 to 476 in the amino acid sequence shown under SEQ ID NO 18, apart from that the amino acid Tat position 327 in SEQ ID NO 18 is substituted by Q and the amino acid Cat position 384 in SEQ ID NO 18 is substituted by S;
c) proteins having the amino acid sequence from positions 1 to 476 in the amino acid sequence shown under SEQ ID NO 18, apart from that the amino acid E at position 326 in SEQ ID NO 18 is substituted by Q and the amino acid Tat position 327 in SEQ ID NO 18 is substituted by Q;
d) proteins having the amino acid sequence from positions 1 to 476 in the amino acid sequence shown under SEQ ID NO 18, apart from that the amino acid Tat position 327 in SEQ ID NO 18 is substituted by Q;
e) proteins having the amino acid sequence from positions 1 to 476 in the amino acid sequence shown under SEQ ID NO 18, apart from hat the amino acid E at position 326 in SEQ ID NO 18 is substituted by F and the amino acid Tat position 327 in SEQ ID NO 18 is substituted by Q;
f) proteins having the amino acid sequence from positions 1 to 476 in the amino acid sequence shown under SEQ ID NO 18, apart from that the amino acid Tat position 327 in SEQ ID NO 18 is substituted by C;
g) proteins having the amino acid sequence from positions 1 to 476 in the amino acid sequence shown under SEQ ID NO 18, apart from that the amino acid Tat position 327 in SEQ ID NO 18 is substituted by I;
h) proteins having the amino acid sequence from positions 1 to 476 in the amino acid sequence shown under SEQ ID NO 18, apart from that the amino acid Tat position 327 in SEQ ID NO 18 is substituted by M;
i) proteins having the amino acid sequence from positions 1 to 476 in the amino acid sequence shown under SEQ ID NO 18, apart from that the amino acid F at position 164 in SEQ ID NO 18 is substituted by Y;
j) proteins having the amino acid sequence from positions 1 to 476 in the amino acid sequence shown under SEQ ID NO 18, apart from that the amino acid F at position 164 in SEQ ID NO 18 is substituted by S;
k) proteins having the amino acid sequence from positions 1 to 476 in the amino acid sequence shown under SEQ ID NO 18, apart from that the amino acid Tat position 327 in SEQ ID NO 18 is substituted by V;
l) proteins having the amino acid sequence from positions 1 to 476 in the amino acid sequence shown under SEQ ID NO 18, apart from that the amino acid Tat position 409 in SEQ ID NO 18 is substituted by R;
m) proteins having the amino acid sequence from positions 1 to 476 in the amino acid sequence shown under SEQ ID NO 18, apart from that the amino acid Tat position 327 in SEQ ID NO 18 is substituted by S;
n) proteins having the amino acid sequence from positions 1 to 476 in the amino acid sequence shown under SEQ ID NO 18, apart from that the amino acid V at position 271 in SEQ ID NO 18 is substituted by I;
o) proteins having the amino acid sequence from positions 1 to 476 in the amino acid sequence shown under SEQ ID NO 18, apart from that the amino acid S at position 329 in SEQ ID NO 18 is substituted by G;
p) proteins having the amino acid sequence from positions 1 to 476 in the amino acid sequence shown under SEQ ID NO 18, apart from that the amino acid Tat position 409 in SEQ ID NO 18 is substituted by P;
q) proteins having the amino acid sequence from positions 1 to 476 in the amino acid sequence shown under SEQ ID NO 18, apart from that the amino acid L at position 414 in SEQ ID NO 18 is substituted by M;
r) proteins having the amino acid sequence from positions 1 to 476 in the amino acid sequence shown under SEQ ID NO 18, apart from that the amino acid Q at position 165 in SEQ ID NO 18 is substituted by K;
s) proteins having the amino acid sequence from positions 1 to 476 in the amino acid sequence shown under SEQ ID NO 18, apart from that the amino acid L at position 414 in SEQ ID NO 18 is substituted by R;
t) proteins having the amino acid sequence from positions 1 to 476 in the amino acid sequence shown under SEQ ID NO 18, apart from that the amino acid L at position 414 in SEQ ID NO 18 is substituted by H;
u) proteins having the amino acid sequence from positions 1 to 476 in the amino acid sequence shown under SEQ ID NO 18, apart from that the amino acid Q at position 165 in SEQ ID NO 18 is substituted by C;
v) proteins having the amino acid sequence from positions 1 to 476 in the amino acid sequence shown under SEQ ID NO 18, apart from that the amino acid Tat position 327 in SEQ ID NO 18 is substituted by V;
w) proteins having the amino acid sequence from positions 1 to 476 in the amino acid sequence shown under SEQ ID NO 18, apart from that the amino acid F at position 164 in SEQ ID NO 18 is substituted by C;
x) proteins having the amino acid sequence from positions 1 to 476 in the amino acid sequence shown under SEQ ID NO 18, apart from that the amino acid Tat position 409 in SEQ ID NO 18 is substituted by K
y) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequences as defined under a), b), c), d), e), f), g), h), i), j), k), l), m), n), o), p), q), r), s), t), u), v), w) or x) given that each amino acid position as defined under a), b), c), d), e), f), g), h), i), j), k), l), m), n), o), p), q), r), s), t), u), v), w) or x), respectively, is also present at the corresponding amino acid position in the amino acid sequences of the protein sequence having at least 60 preferably 70%, more preferably 80%, further more preferably 90%, even more preferably 95%, even further more preferably 96%, particular preferably 97%, most preferably 98% or especially preferably 99% identity with any of the amino acid sequences as defined under each of a), b), c), d), e), f), g), h), i), j), k), l), m), n), o), p), q), r), s), t), u), v), w) or x).

As an embodiment of the invention, preferred proteins having the activity of an ω-TA variant comprising further amino acid modifications are those proteins defined under items a), b), c), d), e), f), g), h), i), j), k), l), m), n), o) and p) defined just above, more preferred are those proteins defined under items a), b), c), d), e), f), g) and h) defined just above and most preferred are those proteins defined under items a), b) and c) defined just above.

Table 2 summarizes the additional amino acid modifications present in the amino acid sequence of ω-TAs comprising further amino acid modifications in comparison to the amino acid sequence shown under SEQ ID NO 18 (from positions 1 to 476).

TABLE 2
One further embodiment of the invention concerns nucleic acid molecules encoding
a protein according to the invention.

| ω-TA variant | Amino | | | Amino | | |
|---|---|---|---|---|---|---|
| comprising | acid | Amino acid in | | acid | Amino acid in | |
| further amino | position | further | | Position | further | |
| acid | in SEQ ID | modified | SEQ ID | in SEQ ID | modified | SEQ ID |
| modifications | NO 18 | variant | NO 18 | NO 18 | variant | NO 18 |
| T327Q, S166G | 327 | Q | T | 166 | G | S |
| T327Q, C384S | 327 | Q | T | 384 | S | C |
| T327Q, E326Q | 327 | Q | T | 326 | Q | E |
| T327Q | 327 | Q | T | | | |
| T327Q, E326F | 327 | Q | T | 326 | F | E |
| T327C | 327 | C | T | | | |
| T327I | 327 | I | T | | | |
| T327M | 327 | M | T | | | |
| F164Y | 164 | Y | F | | | |
| F164S | 164 | S | F | | | |
| T327V | 327 | V | T | | | |
| T409R | 409 | R | T | | | |
| T327S | 327 | S | T | | | |
| V271I | 271 | I | V | | | |
| S329G | 329 | G | S | | | |
| T409P | 409 | P | T | | | |
| L414M | 414 | M | L | | | |
| Q165K | 165 | K | Q | | | |
| L414R | 414 | R | L | | | |
| L414H | 414 | H | L | | | |
| Q165C | 165 | C | Q | | | |
| T327V | 327 | V | T | | | |
| F164C | 164 | C | F | | | |
| T409K | 409 | K | T | | | |

Nucleic acid molecules according to the invention can be any kind of nucleic acid, as long as the nucleic acid encodes a protein according to the invention. The nucleic acids can be ribonucleic nucleic acid molecules (e.g. RNA, mRNA) or deoxyribonucleic nucleic acid molecules (DNA, including genomic DNA which may or may not comprise introns and coding DNA).

Of particular interest for the invention are nucleic acid molecules encoding a protein having the activity of an ω-TA comprising the amino acid sequence as shown from positions 1 to 476 under SEQ ID NO 18.

The invention therefore also concerns nucleic acid molecules encoding a protein having the activity of an ω-TA selected from the group consisting of
a) nucleic acid molecules comprising the nucleic acid sequence from positions 1 to 1428 in the nucleic acid sequence shown under SEQ ID NO 17;
b) nucleic acid molecules encoding a protein comprising the amino acid sequence from position 1 to 476 in the amino acid sequence shown under SEQ ID NO 18;
c) nucleic acid molecules having at least 60%, preferably 70%, more preferably 80%, further more preferably 90%, even more preferably 95%, even further more preferably 96%, particular preferably 97%, most preferably 98% or especially preferably 99% identity with the nucleic acid sequence from positions 1 to 1428 in the nucleic acid sequence as shown under SEQ ID NO 17 given that the codon corresponding to nucleotide positions 73 to 75 in SEQ ID NO 17 has the nucleotide sequence mgn and the codon corresponding to nucleotide positions 190 to192 in SEQ ID NO 17 has the nucleotide sequence ath and the codon corresponding to nucleotide positions 262 to 264 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 493 to 495 in SEQ ID NO 17 has the nucleotide sequence mgn and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 589 to 591 in SEQ ID NO 17 has the nucleotide sequence gcn and the codon corresponding to nucleotide positions 559 to 561 in SEQ ID NO 17 has the nucleotide sequence aay and the codon corresponding to nucleotide positions 715 to 717 in SEQ ID NO 17 has the nucleotide sequence ccn and the codon corresponding to nucleotide positions 979 to 981 in SEQ ID NO 17 has the nucleotide sequence acn and the codon at nucleotide positions 982 to 984 in SEQ ID NO 17 has the nucleotide sequence ggn and the codon corresponding to nucleotide positions 1150 to 1152 in SEQ ID NO 17 has the nucleotide sequence tgy and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn;
d) nucleic acid molecules having at least 60%, preferably 70%, more preferably 80%, further more preferably 90%, even more preferably 95%, even further more preferably 96%, particular preferably 97%, most preferably 98% or especially preferably 99% identity with the nucleic acid sequence from positions 1 to 1428 in the nucleic acid sequence as shown under SEQ ID NO 17, given that the codon corresponding to nucleotide positions 4 to 6 in SEQ ID NO 17 has the nucleotide sequence ggn and the codon corresponding to nucleotide positions 73 to 75 in SEQ ID NO 17 has the nucleotide sequence mgn and the codon the codon corresponding to nucleotide positions 136 to 138 in SEQ ID NO 17 has the nucleotide sequence atg and the codon the codon corresponding to nucleotide positions 142 144 in SEQ ID NO 17 has the nucleotide sequence ggn and the codon corresponding to nucleotide positions 178 to 180 in SEQ ID NO 17 has the nucleotide sequence tay and the codon corresponding to nucleotide positions 190 to 192 in SEQ ID NO 17 has the nucleotide sequence ath and the codon corresponding to nucleotide positions 205 to 207 in SEQ ID NO 17 has the nucleotide sequence car and the codon corresponding to nucleotide positions 262 to 264 in SEQ ID NO 17 has the nucleotide sequence gcn and the codon corresponding to nucleotide positions 268 to 270 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 490 to 492 in SEQ ID NO 17 has the nucleotide sequence tty and the codon corresponding to nucleotide positions 493 to 495 in SEQ ID NO 17 has the nucleotide sequence car and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 553 to 555 in SEQ ID NO 17 has the nucleotide sequence tay and the codon corresponding to nucleotide positions 556 to 558 in SEQ ID NO 17 has the nucleotide sequence aay and the codon corresponding to nucleotide positions 559 to 561 in SEQ ID NO 17 has the nucleotide sequence aay and the codon corresponding to nucleotide positions 583 to 585 in SEQ ID NO 17 has the nucleotide sequence ccn and the codon corresponding to nucleotide positions 589 to 591 in SEQ ID NO 17 has the nucleotide sequence gcn and the codon corresponding to nucleotide positions 604 to 606 in SEQ ID NO 17 has the nucleotide sequence aay and the codon corresponding to nucleotide positions 613 to 615 in SEQ ID NO 17 has the nucleotide sequence tgy and the codon corresponding to nucleotide positions 715 to 717 in SEQ ID NO 17 has the nucleotide sequence ccn and the codon corresponding to nucleotide positions 724 to 726 in SEQ ID NO 17 has the nucleotide sequence gtn and the codon corresponding to nucleotide positions 733 to 735 in SEQ ID NO 17 has the nucleotide sequence acn and the codon corresponding to nucleotide positions 754 to 756 in SEQ ID NO 17 has the nucleotide sequence ath and the codon corresponding to nucleotide positions 763 to 765 in SEQ ID NO 17 has the nucleotide sequence ath and the codon corresponding to nucleotide positions 802 to 804 in SEQ ID NO 17 has the nucleotide sequence aay and the codon corresponding to nucleotide positions 931 to 933 in SEQ ID NO 17 has the nucleotide sequence gtn and the codon corresponding to nucleotide positions 952 to 954 in SEQ ID NO 17 has the nucleotide sequence gcn and the codon corresponding to nucleotide positions 964 to 966 in SEQ ID NO 17 has the nucleotide sequence aar and the codon corresponding to nucleotide positions 979 to 981 in SEQ ID NO 17 has the nucleotide sequence acn and the codon corresponding to nucleotide positions 982 to 984 in SEQ ID NO 17 has the nucleotide sequence ggn and the codon corresponding to nucleotide positions 1057 to 1059 in SEQ ID NO 17 has the nucleotide sequence ytn and the codon corresponding to nucleotide positions 1075 to 1077 in SEQ ID NO 17 has the nucleotide sequence aay and the codon corresponding to nucleotide positions 1150 to 1152 in SEQ ID NO 17 has the nucleotide sequence tay and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1225 to 1227 in SEQ ID NO 17 has the nucleotide sequence acn and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn and the codon corresponding to nucleotide positions 1270 to 1272 in SEQ ID NO 17 has the nucleotide sequence gar and the codon corresponding to nucleotide positions 1306 to 1308 in SEQ ID NO 17 has the nucleotide sequence gtn and the codon corresponding to nucleotide positions 1354 to 1356 in SEQ ID NO 17 has the nucleotide sequence ggn;
e) nucleic acid molecules hybridizing with the complemantary strand of the nucleic acid molecules defined under a), b), c) or d), given that the codon corresponding to nucleotide positions 73 to 75 in SEQ ID NO 17 has the nucleotide sequence mgn and the codon corresponding to nucleotide positions 190 to192 in SEQ ID NO 17 has the nucleotide sequence ath and the codon corresponding to nucleotide positions 262 to 264 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 493 to 495 in SEQ ID NO 17 has the nucleotide sequence mgn and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 559 to 561 in SEQ ID NO 17 has the nucleotide sequence aay and the codon corresponding to nucleotide positions 715 to 717 in SEQ ID NO 17 has the nucleotide sequence ccn and the codon corresponding to nucleotide positions 979 to 981 in SEQ ID NO 17 has the nucleotide sequence acn and the codon at nucleotide positions 982 to 984 in SEQ ID NO 17 has the nucleotide sequence ggn and the codon corresponding to nucleotide positions 1150 to 1152 in SEQ ID NO 17 has the nucleotide sequence tgy and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn;
f) nucleic acid molecules hybridizing with the complemantary strand of the nucleic acid molecules defined under a), b), c) or d) given that the codon corresponding to nucleotide positions 4 to 6 in SEQ ID NO 17 has the nucleotide sequence ggn and the codon corresponding to nucleotide positions 73 to 75 in SEQ ID NO 17 has the nucleotide sequence mgn and the codon the codon corresponding to nucleotide positions 136 to 138 in SEQ ID NO 17 has the nucleotide sequence atg and the codon the codon corresponding to nucleotide positions 142 144 in SEQ ID NO 17 has the nucleotide sequence ggn and the codon corresponding to nucleotide positions 178 to 180 in SEQ ID NO 17 has the nucleotide sequence tay and the codon corresponding to nucleotide positions 190 to 192 in SEQ ID NO 17 has the nucleotide sequence ath and the codon corresponding to nucleotide positions 205 to 207 in SEQ ID NO 17 has the nucleotide sequence car and the codon corresponding to nucleotide positions 262 to 264 in SEQ ID NO 17 has the nucleotide sequence gcn and the codon corresponding to nucleotide positions 268 to 270 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 490 to 492 in SEQ ID NO 17 has the nucleotide sequence tty and the codon corresponding to nucleotide positions 493 to 495 in SEQ ID NO 17 has the nucleotide sequence car and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 553 to 555 in SEQ ID NO 17 has the nucleotide sequence tay and the codon corresponding to nucleotide positions 556 to 558 in SEQ ID NO 17 has the nucleotide sequence aay and the codon corresponding to nucleotide positions 559 to 561 in SEQ ID NO 17 has the nucleotide sequence aay and the codon corresponding to nucleotide positions 583 to 585 in SEQ ID NO 17 has the nucleotide sequence ccn and the codon corresponding to nucleotide positions 589 to 591 in SEQ ID NO 17 has the nucleotide sequence gcn and the codon corresponding to nucleotide positions 604 to 606 in SEQ ID NO 17 has the nucleotide sequence aay and the codon corresponding to nucleotide positions 613 to 615 in SEQ ID NO 17 has the nucleotide sequence tgy and the codon corresponding to nucleotide positions 715 to 717 in SEQ ID NO 17 has the nucleotide sequence ccn and the codon corresponding to nucleotide positions 724 to 726 in SEQ ID NO 17 has the nucleotide sequence gtn and the codon corresponding to nucleotide positions 733 to 735 in SEQ ID NO 17 has the nucleotide sequence acn and the codon corresponding to nucleotide positions 754 to 756 in SEQ ID NO 17 has the nucleotide sequence ath and the codon corresponding to nucleotide positions 763 to 765 in SEQ ID NO 17 has the nucleotide sequence ath and the codon corresponding to nucleotide positions 802 to 804 in SEQ ID NO 17 has the nucleotide sequence aay and the codon corresponding to nucleotide positions 931 to 933 in SEQ ID NO 17 has the nucleotide sequence gtn and the codon corresponding to nucleotide positions 952 to 954 in SEQ ID NO 17 has the nucleotide sequence gcn and the codon corresponding to nucleotide positions 964 to 966 in SEQ ID NO 17 has the nucleotide sequence aar and the codon corresponding to nucleotide positions 979 to 981 in SEQ ID NO 17 has the nucleotide sequence acn and the codon corresponding to nucleotide positions 982 to 984 in SEQ ID NO 17 has the nucleotide sequence ggn and the codon corresponding to nucleotide positions 1057 to 1059 in SEQ ID NO 17 has the nucleotide sequence ytn and the codon corresponding to nucleotide positions 1075 to 1077 in SEQ ID NO 17 has the nucleotide sequence aay and the codon corresponding to nucleotide positions 1150 to 1152 in SEQ ID NO 17 has the nucleotide sequence tay and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1225 to 1227 in SEQ ID NO 17 has the nucleotide sequence acn and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn and the codon corresponding to nucleotide positions 1270 to 1272 in SEQ ID NO 17 has the nucleotide sequence gar and the codon corresponding to nucleotide positions 1306 to 1308 in SEQ ID NO 17 has the nucleotide sequence gtn and the codon corresponding to nucleotide positions 1354 to 1356 in SEQ ID NO 17 has the nucleotide sequence ggn;
g) nucleic acid molecules deviating from the nucleic acid molecules defined under a), b), c), d), e) or f) due to degeneracy of the genetic code;
h) nucleic acid molecules encoding a protein having at least 60%, preferably 70%, more preferably 80%, further more preferably 90%, even more preferably 95%, even further more preferably 96%, particular preferably 97%, most preferably 98% or especially preferably 99% identity with the amino acid sequence from positions 1 to 476 as shown under SEQ ID NO 18 given that the amino acids corresponding to positions 25, 64, 88, 157, 165, 169, 174, 187, 239, 327, 328, 384, 389, 391, 396, 410 and 414 in SEQ ID NO 18 represent those amino acids shown at the respective positions in the amino acid sequence shown under SEQ ID NO 18;
i) nucleic acid molecules encoding a protein having at least 60%, preferably 70%, more preferably 80%, further more preferably 90%, even more preferably 95%, even further more preferably 96%, particular preferably 97%, most preferably 98% or especially preferably 99% identity with the amino acid sequence from positions 1 to 476 as shown under SEQ ID NO 18 given that the amino acids corresponding to positions 2, 25, 46, 48, 60, 64, 69, 88, 90, 157, 164, 165, 169, 174, 185, 186, 187, 195, 197, 202, 205, 239, 242, 245, 252, 255, 268, 311, 318, 322, 327, 328, 353, 359, 384, 389, 391, 396, 409, 410, 414, 424, 436, 452, 475 and 476 in SEQ ID NO 18 represent those amino acids shown at the respective positions in the amino acid sequence shown under SEQ ID NO 18;
j) nucleic acid molecules comprising the nucleic acid sequence from positions from 1 to 1428 in the nucleic acid sequence as shown under SEQ ID NO 16.

SEQ ID NO 16 shows a nucleotide sequence obtained by back-translation of a protein having the amino acid sequence as shown under SEQ ID NO 18, wherein degeneracy of the genetic code is reflected.

SEQ ID NO 17 is a synthetic nucleic acid molecule obtained by substituting the, due to 20 degeneracy of the genetic code flexible nucleotides in SEQ ID NO 16 by specific nucleotides. Both, SEQ ID NO 16 and SEQ ID NO 17 encode a protein having the activity of an ω-TA having the amino acid sequence as shown under SEQ ID NO 18.

In the context of the present invention, the term “hybridizing with” means hybridization under conventional hybridization conditions, preferably under stringent conditions, as described, for example, in Sambrook et al. (Molecular Cloning, A Laboratory Manual, 3rd edition (2001) Cold Spring Harbor Laboratory Press, Cold Spring Harbor, NY. ISBN: 0879695773) or Ausubel et al. (Short Protocols in Molecular Biology, John Wiley & Sons; 5th edition (2002), ISBN: 0471250929). With particular preference, “hybridization” means a hybridization under the 30 following conditions:

hybridization buffer:

2×SSC; 10×Denhardt solution (Fikoll 400+PEG+BSA; ratio 1:1:1); 0.1% SDS; 5 mM EDTA; 50 mM Na2HPO4; 250 μg/ml of herring sperm DNA; 50 μg/ml of tRNA; or

25 M sodium phosphate buffer pH 7.2; 1 mM EDTA; 7% SDS

hybridization temperature:T=65 to 68° C.

wash buffer: 0.1×SSC; 0.1% SDS

wash temperature: T=65 to 68° C.

Nucleic acid molecules which hybridize with nucleic acid molecules coding for a protein having the activity of an ω-TA may originate from any organism; accordingly, they may originate from bacteria, fungi, animals, humans, plants or viruses.

Nucleic acid molecules which hybridize with nucleic acid molecules coding for a protein having the activity of an ω-TA preferably originate from microorganisms, more preferably from fungi or bacteria, most preferably from bacteria.

Nucleic acid molecules which hybridize with the molecules mentioned may be isolated, for example, from genomic or from cDNA libraries. Such nucleic acid molecules can be identified and isolated using the nucleic acid molecules described herein or they can be identified and isolated using parts of these molecules or the reverse complements of these molecules, for example by hybridization according to standard methods (see, for example, Sambrook et al., Molecular Cloning, A Laboratory Manual, 3rd edition (2001) Cold Spring Harbor Laboratory Press, Cold Spring Harbor, NY. ISBN: 0879695773; Ausubel et al., Short Protocols in Molecular Biology, John Wiley & Sons; 5th edition (2002), ISBN: 0471250929) or by amplification using PCR.

As hybridization sample for isolating a nucleic acid sequence coding for a protein having the activity of an ω-TA, it is possible to use, for example, nucleic acid molecules having exactly or essentially the nucleic acid sequences from positions 1 to 1431 described under SEQ ID NO 2 or essentially the nucleic acid sequences from positions 1 to 1437 described under SEQ ID NO 5 or essentially the nucleic acid sequences described under SEQ ID NO 8 or essentially the nucleic acid sequences described under SEQ ID NO 11 or essentially the nucleic acid sequences described under SEQ ID NO 14 or essentially the nucleic acid sequences described under SEQ ID NO 17 or fragments of these nucleic acid sequences.

The fragments used as hybridization samples may also be synthetic fragments or oligonucleotides prepared using the customary synthesis techniques, whose sequence is essentially identical to the nucleic acid molecule described in the context of the present invention. Once genes which hybridize with the nucleic acid sequences described in the context of the present invention are identified and isolated, the sequence should be determined and the properties of the proteins coded for by this sequence should be analysed to determine whether they are proteins having the activity of a an ω-TA. Methods of how to determine whether a protein has the activity of a protein having the activity of an ω-TA are known to the person skilled in the art and have been mentioned herein above.

The molecules hybridizing with the nucleic acid molecules described in the context of the present invention comprise in particular fragments, derivatives and allelic variants of the nucleic acid molecules mentioned. In the context of the present invention, the term “derivative” means that the sequences of these molecules differ in one or more positions from the sequences of the nucleic acid molecules described above and are highly identical to these sequences. The differences to the nucleic acid molecules described above may, for example, be due to deletion, addition, substitution, insertion or recombination.

Another embodiment of the invention in respect to nucleic acid molecules encoding proteins having the activity of an ω-TA comprising further amino acid modifications concerns nucleic acid molecules according to the invention encoding proteins having the activity of an ω-TA selected from the group consisting of
a) nucleic acid molecules comprising the nucleic acid sequence from positions 1 to 1428 in the nucleic acid sequences as shown under SEQ ID NO 16 or SEQ ID NO 17, apart from that the codon at nucleotide positions 496 to 498 in SEQ ID NO 16 or SEQ ID NO 17 has the nucleotide sequence ggn and the codon at position at nucleotide positions 979 to 981 in SEQ ID NO 16 or SEQ ID NO 17 has the nucleotide sequence car;
b) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car and the codon at nucleotide positions 1150 to 1152 in SEQ ID NO 16 or SEQ ID NO 17 has the nucleotide sequence wsn;
c) nucleic acid molecules comprising the nucleic acid sequence from positions 1 to 1428 in the nucleic acid sequences as shown under SEQ ID NO 16 or SEQ ID NO 17, apart from that the codon at nucleotide positions 976 to 978 in SEQ ID NO 16 or SEQ ID NO 17 has the nucleotide sequence car and the codon at position at nucleotide positions 979 to 981 in SEQ ID NO 16 or SEQ ID NO 17 has the nucleotide sequence car;
d) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car,
e) nucleic acid molecules comprising the nucleic acid sequence from positions 1 to 1428 in the nucleic acid sequences as shown under SEQ ID NO 16 or SEQ ID NO 17, apart from that the codon at nucleotide positions 976 to 978 in SEQ ID NO 16 or SEQ ID NO 17 has the nucleotide sequence tty and the codon at nucleotide positions 979 to 981 in SEQ ID NO 16 or SEQ ID NO 17 has the nucleotide sequence car,
f) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car,
g) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence ath;
h) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence atg;
i) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence tay;
j) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence wsn;
k) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence gtn;
l) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence mgn;
m) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence wsn;
n) nucleic acid molecules comprising the nucleic acid sequence from positions 1 to 1428 in the nucleic acid sequences as shown under SEQ ID NO 16 or SEQ ID NO 17, apart from that the codon at nucleotide positions 811 to 813 in SEQ ID NO 16 or SEQ ID NO 17 has the nucleotide sequence ath;
o) nucleic acid molecules comprising the nucleic acid sequence from positions 1 to 1428 in the nucleic acid sequences as shown under SEQ ID NO 16 or SEQ ID NO 17, apart from that the codon at nucleotide positions 985 to 987 in SEQ ID NO 16 or SEQ ID NO 17 has the nucleotide sequence ggn;
p) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence ccn;
q) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence atg;
r) nucleic acid molecules comprising the nucleic acid sequence from positions 1 to 1428 in the nucleic acid sequences as shown under SEQ ID NO 16 or SEQ ID NO 17, apart from that the codon at nucleotide positions 493 to 495 in SEQ ID NO 16 or SEQ ID NO 17 has the nucleotide sequence aar;
s) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence mgn;
t) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence cay;
u) nucleic acid molecules comprising the nucleic acid sequence from positions 1 to 1428 in the nucleic acid sequences as shown under SEQ ID NO 16 or SEQ ID NO 17, apart from that the codon at nucleotide positions 493 to 495 in SEQ ID NO 16 or SEQ ID NO 17 has the nucleotide sequence tgy;
v) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence gtn;
w) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence tgy;
x) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence aar;
y) nucleic acid molecules having a nucleic acid sequence having at least 60%, preferably 70%, more preferably 80%, further more preferably 90%, even more preferably 95%, even further more preferably 96%, particular preferably 97%, most preferably 98% or especially preferably 99% identity with any of the nucleic acid sequences as defined under a), b), c), d), e), f), g), h), i), j), k), l), m), n), o), p), q), r), s), t), u), v), w) or x) given that each codon nucleotide sequence as defined in each of a), b), c), d), e), f), g), h), i), j), k), l), m), n), o), p), q), r), s), t), u), v), w) or x), respectively, is also present at the corresponding codon nucleotide position in the nucleic acid sequences having at least 60% preferably 70%, more preferably 80%, further more preferably 90%, even more preferably 95%, even further more preferably 96%, particular preferably 97%, most preferably 98% or especially preferably 99% identity with any of the nucleic acid sequences as defined under each of a), b), c), d), e), f), g), h), i), j), k), l), m), n), o), p), q), r), s), t), u), v), w) or x).

Preferred nucleic acid molecules according to the invention are those nucleic acid molecules defined just above under items a), b), c), d), e), f), g), h), i), j), k), l), m), n), o) and p), more preferred are those nucleic acid molecules defined just above under items a) to k), even more preferred are those nucleic acid molecules defined just above under items a) , b), c), d), e), f), g) and h) and most preferred are those nucleic acid molecules defined just above under items a), b) and c).

The meaning of nucleotide abbreviations a, c, g, t, and those of abbreviations for degenerate nucleotides r, y, s, w, k, m, b, d, h, v, n is derivable herein below from Table 3 under the paragraph sub-titled “Description of the Sequences”. Which amino acids are encoded by codons comprising degenerate nucleotides is derivable herein below from Table 5 under the paragraph sub-titled “Description of the Sequences”.

Furthermore, the invention relates to recombinant nucleic acid molecules comprising a nucleic acid molecule according to the invention.

In conjunction with the present invention, the term “recombinant nucleic acid molecule” is to be understood to mean a nucleic acid molecule, which contains additional sequences in addition to nucleic acid molecules according to the invention, which do not naturally occur in the combination in which they occur in recombinant nucleic acids according to the invention. Here, the abovementioned additional sequences can be any sequences, preferably they are functional or regulatory sequences (promoters, termination signals, enhancers, ribosome binding sites (rbs), leader sequences enhancing transcription, translation or RNA stability, subcellular targeting sequences etc.), particularly preferably they are functional or regulatory sequences that are active in microorganisms, and especially particularly preferably they are regulatory sequences that are active in fungi, in particular yeasts or in bacteria. Methods for the creation of recombinant nucleic acid molecules according to the invention are known to the person skilled in the art, and include genetic methods such as bonding nucleic acid molecules by way of ligation, genetic recombination, or new synthesis of nucleic acid molecules. Those methods are described e.g. in Sambrok et al. (Molecular Cloning, A Laboratory Manual, 3rd edition (2001) Cold Spring Harbour Laboratory Press, Cold Spring Harbour, NY. ISBN: 0879695773) or Ausubel et al. (Short Protocols in Molecular Biology, John Wiley & Sons; 5th edition (2002), ISBN: 0471250929).

In a further embodiment, the recombinant nucleic acid molecules according to the invention comprise a nucleic acid molecule according to the invention which is linked with regulatory sequences, which initiate transcription in prokaryotic or eukaryotic cells.

Regulatory sequences, which initiate transcription” in a cell are also known as promoters.

Information concerning regulatory sequences and plasmids are well known to a person skilled in the art and are described e.g. by the Registry of Standard Biological Parts supported by The International Genetically Engineered Machine (iGEM) Foundation (One Kendall Square, Suite B6104, Cambridge, Mass. 02139, USA) in the world wide web (http://parts.igem.org/Catalog).

Regulatory sequences which initiate transcription in prokaryotic organisms, e.g. E. coli, and in eukaryotic organisms are sufficiently described in literature, in particular such for expression in yeast are described, e.g. Saccharomyces cerevisiae. An overview of various systems for expression for proteins in various host organisms can be found, for example, in Methods in Enzymology 153 (1987), 383-516 and in Bitter et al. (Methods in Enzymology 153 (1987), 516-544) or in Gomes et al. (2016, Advances in Animal and Veterinary Sciences, 4(4), 346) and Baghban et al. (2018, Current Pharmaceutical Biotechnology, 19(6)). Common yeast promoters are pAOX1, pHIS4, pGAL, pScADH2 (Baghban et al., 2018, see above). Common bacterial promoters are T5, T7, rhamnose-inducible, arabinose-inducible, PhoA, artificial trc (trp-lac) promoter as described by Marschall et al. (2017, Appl Microbiol Biotechnol 101, 501-512) and Tegel et al. (2011, FEBS Journal 278, 729-739).

A further embodiment of recombinant nucleic acid molecules of the present invention are vectors or plasmids, which comprise the nucleic acid molecules according to the invention.

Vectors” are commonly understood in the field of molecular biology and herein to represent a nucleic acid sequence or a vehicle comprising a nucleic acid sequence used to transfer genetic material (DNA or RNA) into a target cell. Vectors can be plasmids, e.g. T-DNA or binary vectors for generating transgenic plants, expression vectors for expression of nucleic acid sequences in a host cell, shuttle vectors which are eligible to propagate in different hosts, or vectors can be virus particles or bacteriophages having been modified to deliver foreign genetic material into a host.

“Plasmids” are commonly understood in the field of molecular biology and herein to represent an autonomously self-replicating, often circular DNA molecule which is when present in a host cell separated from the chromosomal DNA.

Nucleic acid molecules according to the invention, recombinant nucleic acid molecules according to the invention, vectors or plasmids according to the invention can be used for production of proteins according to the invention, e.g. by expressing the nucleic acid molecules according to the invention in host cells.

Another embodiment of the invention concerns hosts or host cells comprising or expressing a nucleic acid molecule according to invention or comprising proteins according to the invention or comprising a recombinant nucleic acid molecule according to the invention or comprising a vector according to the invention or comprising a plasmid according to the invention.

Nucleic acid molecules according to the invention encoding a protein having the activity of an ω-TA can be expressed in host cells for e.g. their multiplication or for production of proteins according to the invention. For expression in host cells, nucleic acid molecules according to the invention can be comprised on vectors or plasmids or they can be stably integrated into the genome of a respective host cell. The nucleic acid molecules according to the invention can also be comprised by vectors which support their introduction into host cells.

A further embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case comprising a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein, wherein the protein has the activity of an ω-transaminase.

“Expressing a nucleic acid molecule” shall be understood herein to mean that in case the nucleic acid molecule is RNA or mRNA the nucleic acid molecule is translated into a protein, preferably translated into a protein having the activity of an ω-TA or in case of the nucleic acid molecule is DNA or cDNA it is transcribed (and in case of genomic DNA containing introns is processed) into mRNA, preferably into a mRNA encoding a protein having the activity of an ω-TA and subsequently translated into a protein, preferably translated into a protein having the activity of an ω-TA.

Transcription of a given nucleic acid molecule in a host can be demonstrated by methods known to a person skilled in the art, for example, by detection of specific transcripts (mRNA) of foreign nucleic acid molecules by Northern blot analysis or RT-PCR.

Whether hosts or host cells comprise a given protein or comprise a protein which is derived from expressing a nucleic acid molecule can be determined by methods known to a person skilled in the art, for example, by immunological methods, such as Western blot analysis, ELISA (Enzyme Linked Immuno Sorbent Assay) or RIA (Radio Immune Assay). The person skilled in the art is familiar with methods for preparing antibodies which react specifically with a certain protein, i.e. which bind specifically to a certain protein (see, for example, Lottspeich and Zorbas (eds.), 1998, Bioanalytik, Spektrum akad, Verlag, Heidelberg, Berlin, ISBN 3-8274-0041-4). Some companies (Thermo Fisher Scientific, 168 Third Avenue, Waltham, Mass. USA 0245; GenScript, 60 Centennial Ave., Piscataway, N.J. 08854, USA) offer the preparation of such antibodies as an order service.

Furthermore, a person skilled in the art can test if a host or host cell comprises a protein according to the invention by detecting (additional) activity of proteins having the activity of an ω-TA in a respective host cell. Preferably activity of proteins having additional activity of an ω-TA in a respective host cell is detected by comparing the activities of ω-TAs of a host cell according to the invention with the respective activity of host cell not comprising a protein according to the invention.

Testing if a protein has the activity of an ω-TA can be done as described herein above.

Host or host cells according to the invention can be produced by a person skilled in the art by known methods for genetically modifying or transforming organisms.

A further subject of the present invention therefore is a host or host cell according to the invention, particularly a prokaryotic or eukaryotic host or host cell, which is genetically modified (or transformed) with a nucleic acid molecule according to the invention or with a recombinant nucleic acid molecule according to the invention or with a vector according to the invention or a plasmid according to the invention. Preferably the genetically modified (transformed) host or host cell according to the invention expresses a protein having the activity of an co-transaminase, more preferably, the genetically modified (transformed) host or host cell according to the invention expresses a protein according to the invention.

“Genetically modified with a nucleic acid molecule” or “transformed with a nucleic acid molecule” shall be understood herein to mean that a nucleic acid molecule is or was introduced into a host or host cell by technical and/or non-naturally occurring means, preferably by technical methods in the field of molecular biology, biotechnology or genetic modification.

Descendants, offspring or progeny of hosts or host cells according to the invention are also an embodiment of the invention, preferably these descendants, offspring or progeny comprise a nucleic acid molecule according to the invention or comprise a recombinant nucleic acid molecule according to the invention or comprise a vector according to the invention or comprise a plasmid according to the invention or comprise a protein according to the invention, more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of an ω-TA, even more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of an ω-TA according to the invention.

The host or host cell according to the invention can be a host or host cell from any prokaryotic or eucaryotic organism. The hosts or host cells can be bacteria or bacteria cells (e.g. E. coli, bacteria of the genus Bacillus, in particular Bacillus subtilis, Agrobacterium, particularly Agrobacterium tumefaciens or Agrobacterium rhizogenes, Pseudomonas, particularly Pseudomonas fluorescens, Streptomyces spp, Rhodococcus spp, in particular Rhodococcus rhodochrous, Vibrio natrigens, Corynebacterium, particularly Corynebacterium glutamicum) or fungi or fungal cells (e.g. Agaricus, in particular Agaricus bisporus, Aspergillus, Trichoderma or yeasts, particularly S. cerevisiae, Pichia ssp. like P. pastoris), as well as plants or plant cells or they can be animals or animal cells.

Preferred host cells according to the invention are cells of microorganisms. Within the framework of the present patent application, this is understood to include all bacteria and all protists (e.g. fungi, particularly yeasts and algae), as they are defined in Schlegel “General Microbiology ” (Georg Thieme Publishing House (1985), 1-2), for example.

In respect to microorganisms, the hosts or host cells according to the invention are preferably bacteria/bacteria cells or yeast/yeast cells, most preferably they are bacteria/bacteria cells. Concerning bacteria/bacteria cells, the hosts or host cells according to the invention are preferably Bacillus species/Bacillus species cells or Escherichia coli/Escherichia coli cells cells most preferably Escherichia coli/Escherichia coli cells.

Alternatively, Pseudomonas, particularly Pseudomonas fluorescens, Streptomyces spp, Rhodococcus spp, in particular Rhodococcus rhodochrous, Vibrio spp, particularly Vibrio natrigens, Corynebacterium, particularly Corynebacterium glutamicum or others can be hosts or host cells according to the invention.

A preferred embodiment of the invention concerns hosts or host cells according to the invention comprising a nucleic acid molecule according to the invention, wherein the nucleic acid molecule according to the invention is characterized in that the codons of said nucleic acid molecule are changed such that they are adapted to the frequency of use of the codons of the host or a host cell, respectively.

Host cells according to the invention can be used for production of proteins according to the invention. Proteins according to the invention can be used in methods for production of enantiomerically enriched or nearly enantiomerically pure amines from a carbonyl (acceptor) in the presence of an amine (donor).

The reaction catalysed in methods for production of enantiomerically enriched or nearly enantiomerically pure amines by a protein according to the invention formally can be described herein above by general equation (I).

Another embodiment of the invention therefore concerns a method for the production of an amine comprising the steps of
a) providing an amine acceptor molecule;
b) providing an amine donor molecule;
c) contacting the amine acceptor molecule provided in step a) and the amine donor molecule provided in step b) with a protein according to the invention;
d) optionally, obtaining the amine.

A preferred embodiment of the method according to the invention for production of an amine is a method for production of an aliphatic amine (including but not limited to linear, branched or cyclic alkan amines, alken amines, alkyn amines) or is a method for production of an aryl amine or is a method for production of an amino acid, more preferably a method for production of an α-amino acid, further more preferably a method for production of a branched α-amino acid, an aromatic α-amino acid or an aromatic α-amino acid comprising substituted phenyl groups, most preferably a method for production of the amino acids norvaline, leucine, phenylalanine or tyrosine.

In respect to the ω-TA variants according to the invention comprising further amino acid modifications the method according to the invention for production of an amine preferably is a method for production of a phosphorous comprising aliphatic amine (including but not limited to a phosphorous comprising linear, branched or cyclic alkan amine, alken amine, alkyn amine) or is a method for production of a phosphorous comprising aryl amine or is a method for production of a phosphorous comprising amino acid, more preferably a method for production of a phosphorous comprising α-amino acid, further more preferably a method for production of a phosphorous comprising branched α-amino acid, a phosphorous comprising aromatic α-amino acid or a phosphorous comprising aromatic α-amino acid comprising substituted phenyl groups, even more preferably a method for production of a phosphorous comprising α-amino acid, even further more preferably a method for production of an α-amino acid comprising a methyl substituted phosphorous, most preferably a method for production of glufosinate.

The amine acceptor molecule in step a) of the method according to the invention for production of an amine is a carbonyl group comprising molecule which accepts an amino group from an amine donor molecule, whereby the carbonyl group of the acceptor molecule becomes an amine.

Preferably, the amine acceptor molecule in step a) of the method according to the invention for production of an amine is an aliphatic ketone (including but not limited to linear, branched or cyclic alkanones, alkenones, alkynones) or is an aryl ketone or is a keto acid, more preferably it is a keto acid, further more preferably it is an a-keto acid, most preferably the amine acceptor molecule is selected from the group consisting of 2-oxovaleric acid, 4-methyl-2-oxovaleric acid, 15 phenylpyruvic acid or 4-hydroxyphenylpyruvic acid.

In respect to protein ω-TA variants according to the invention comprising further amino acid modifications the amine acceptor molecule in step a) of the method according to the invention for production of an amine preferably is a phosphorous containing aliphatic ketone (including but not limited to linear, branched or cyclic alkanones, alkenones, alkynones) or a phosphorous containing aryl ketone or a phosphorous containing keto acid, more preferably the amine acceptor molecule is a phosphorous comprising keto acid, further more preferably the amine acceptor molecule is a phosphorous comprising a-keto acid, even more preferably a methyl substituted phosphorous comprising a-keto acid, most preferably the amine acceptor molecule in step a) is 4-[hydroxy(methyl)phosphoryl]-2-oxobutanoic acid.

Preferably, the amine acceptor molecule in step a) of the method according to the invention for production of an amine is provided in an amount of between 30 g/l (gram per litre) to 300 g/l, more preferably between 30 g/l to 250 g/l, even more preferably between 40 g/l to 250 g/l, further more preferably between 50 g/l to 250 g/l.

The amine donor molecule in step b) of the method according to the invention for production of an amine is an amine group comprising molecule which donates an amine group to the amine acceptor molecule, thereby an amine group of the amine donor molecule becoming a carbonyl group.

The amine donor molecule in step b) of the method according to the invention for production of an amine can be a chiral, pro-chiral or non-chiral amine, preferably the amine donor molecule is a chiral, pro-chiral or non-chiral, respectively, alkyl- or aryl- or aryl-alkyl amine, more preferably the amine donor molecule is an amino acid or a an alkyl-amine.

In respect to alkyl- or aryl amines preferred amino donor molecule to be used in step b) of the method for production of an amine according to the invention are β-alanine, 1-propylamine, (racemic-) 2-butylamine, 6-aminohexanoic acid, isopropylamine, benzylamine, methylbenzylamine, 1-aminoindan, 1-methyl-3-phenylpropylamine.

In case the amino donor is a non-chiral amino acid, glycine is a preferred amino donor molecule to be provided in step b) of the method for production of an amine according to the invention. In case the amino donor in step b) of the method for production of an amine according to the invention is a chiral amino acid, the amino acid preferably is represented by its (S)-enantiomer. Preferred amino acid donor molecules having (S)-configuration to be provided in step b) of the method for production of an amine according to the invention are (S)-methylbenzylamine, (S)-1-aminoindan, (S)-1-methyl-3-phenylpropylamine (S)-asparatic acid, (S)-asparagine, (S)-alanine, (S)-glutamine, (S)-glutamic acid, (S)-ornithine, (S)-phosphoserine, (S)-phenylalanine, (S)-leucine, (S)-tyrosine, (S)-norvaline.

The most preferred amino donor molecule to be provided in step b) of the method for production of an amine according to the invention is isopropylamine.

Isopropylamine when used as an amino donor molecule in the methods according the invention, is converted by the action of an ω-TA into acetone. Acetone is a volatile compound leading to the advantage that it evaporates at relatively low temperatures. This allows removing the acetone produced by the ω-TA from the reaction mixture during the reaction taking place leading to the advantageous effect that the equilibrium of the reaction is shifted towards the amine produced by the method for production of an amine according to the invention. This allows obtaining the desired amine in high amounts as the reverse reaction catalyzed by ω-TA is reduced due to lack of one reaction partner.

Preferably, the amine donor molecule in step b) of the method according to the invention for production of an amine is provided in an amount of between 10 g/l (gram per litre) to 250 g/l, more preferably between 15 g/l to 200 g/l, further more preferably between 17 g/l to 180 g/l.

In step c) of the method for the production of an amine according to the invention the amine acceptor molecule provided in step a) and the amine donor molecule provided in step b) are contacted with a protein according to the invention preferably in solution. The solution can be an aqueous solution comprising only water but it also can be a solution comprising water and organic solvents. In case of contacting the protein according to the invention in step c) of the method for the production of an amine according to the invention with an amine acceptor molecule provided in step a) and an amine donor molecule provided in step b) in an aqueous solutions comprising an organic solvent, the organic solvent is preferably selected from DMSO (di-methyl sulfoxide), DMAc (dimethylacetamide), DMF (dimethylformamide), acetonitrile, toluene, tert-butylmethylether, hexane, heptane. Most preferred are DMSO, DMAc and toluene.

Preferably the aqueous solutions comprising an organic solvent comprise the organic solvent in an amount of up to 10%, more preferably of up to 20%, further more preferably of up to 30%, even more preferably of up to 40%, most preferably up to 50%.

Using aqueous solutions comprising an organic solvent has the advantage that in case the amine acceptor molecule provided in step a) and/or the amine donor molecule provided in step b) of the method for the production of an amine according to the invention has low solubility their respective solubility can be improved, leading to higher amounts of substrates available for the ω-TA. This leads to higher reaction velocity, meaning producing the desired amine in higher amounts in smaller volumes and shorter time, thus improving space-time yield.

In case the protein according to the invention is contacted in step c) of the method for the production of an amine according to the invention with an amine acceptor molecule provided in step a) and an amine donor molecule provided in step b) in an aqueous solution the solution preferably comprises a buffer system for adjusting the pH. Preferred buffer systems are those comprising TRIS-HCI, MOPS, HEPES, TRIS, Bicine.

Preferably the pH of the aqueous solution in which the protein according to invention is contacted in step c) of the method for the production of an amine according to the invention with an amine acceptor molecule provided in step a) and an amine donor molecule provided in step b) is adjusted to a value of between pH 4 to pH 11, more preferably to a value of between pH 5 to pH 10, further more preferably to a value of between pH 6 to pH 10, even more preferably to a value of between pH 7 to pH 10, even further more preferably to a value of between pH 8 to 30 pH 10, most preferably to a value of between pH 8.5 to pH 9.5.

Preferably contacting the amine acceptor molecule provided in step a) and the amine donor molecule provided in step b) with a protein according to the invention in step c) of the method according to the invention for production of an amine takes place at a temperature of between 10° C. and 60° C., more preferably between 20° C. and 60° C., further more preferably between 25° C. and 55° C., even more preferably between 30° C. and 50° C., even further more preferably between 30° C. and 45° C., most preferably between 34° C. and 42° C.

The amine acceptor molecule provided in step a) and the amine donor molecule provided in step b) is contacted with a protein according to the invention in step c) of the method for production of an amine according to the invention for a time sufficient, to produce an amine. Preferably the amine acceptor molecule provided in step a) and the amine donor molecule provided in step b) is contacted with a protein according to the invention in step c) of the method according to the invention for production of an amine for 5 hour to 48 hours, more preferably 5 hours to 36 hours, further more preferably for 5 hours to 30 hours, even more preferably for 5 hours to 24 hours, even further more preferably for 5 hours to 18 hours, most preferably for 5 hours to 14 hours and in particular preferably for 5 hours to 13 hours.

For contacting the protein according to the invention in step c) of the method for production of an amine according to the invention with an amine acceptor molecule provided in step a) and the amine donor molecule provided in step b) the protein can be contacted with an amine acceptor molecule and an amine donor molecule in different forms, preferably the protein is contacted with an amine acceptor molecule and an amine donor molecule in partially purified form or the protein is contacted with an amine acceptor molecule and an amine donor molecule in purified form or the protein is present in a crude cell extract when contacted with an amine acceptor molecule and an amine donor molecule or the protein is contacted with an amine acceptor molecule and an amine donor molecule when present as a component of a living or non-living host cell.

If the protein is contacted in step c) of the method for production of an amine according to the invention with an amine acceptor molecule and an amine donor molecule as a component of a host cell the host cells can be those comprising the culture medium which was used for cultivating the host cells or the host cells can be free from the culture medium the host cells were cultivated in, or the host cells can have been (further) processed, preferably the hosts cells are nearly free from the culture medium the host cells were cultivated in, more preferably the host cells have been (further) processed, even more preferably the hosts cells are nearly free from the culture medium the host cells were cultivated in and the host cells have been (further) processed.

“Crude cell extract” shall mean herein an extract obtained by destruction of a living cell comprising all or substantially all inorganic or organic matter (including further proteins and/or nucleic acid molecules) present in the cell.

“Partially purified” shall mean herein a protein containing composition comprising (only) parts of the total inorganic or organic matter (including further proteins and/or nucleic acid molecules) present in a living cell expressing the protein.

A partially purified extract can e.g. be obtained by fractionation of organic or inorganic matter from a crude cell extract by commonly known means like centrifugation, filtration, any type of chromatographic separation, dialysis etc. Fractionation of a crude cell extract can be performed repeatedly using the same or different fractionation methods and can include precipitation steps.

“Purified” shall mean herein a protein whose specific activity (the activity of the protein present in the fraction dry weight divided by the total amount of the material, in particular other proteins in the fraction dry weight) cannot be increased by further fractionation or purification steps.

It is self-evident from the commonly accepted definition given above for the term “purified” that “purified” can but in most cases does not mean a protein being totally free of any further inorganic and/or organic compounds. Preferably purified shall mean herein that the protein according to the invention represents at least 95%, more preferably at least 96%, further more preferably at least 97%, even more preferably at least 98%, even further more preferably at least 99%, most preferably at least 99.5% of the total amount of the dry weight material comprising the protein.

The term “living cell” shall mean herein a cell which is able to grow and/or reproduce.

The term “non-living cell” shall mean herein a cell which is not able to grow and/or reproduce. Non-living cells, although not able to reproduce and/or grow any more, however still show enzymatic activity, in respect to the present application, in particular activity of a protein according to the invention having the activity of an ω-TA.

The term “free from the culture medium” as used herein means that the culture medium used for cultivation a (host) cell has been removed, e.g. by means of centrifugation and/or filtration.

It is self-evident from the commonly accepted understanding given above for the term “free from the culture medium” can but in most cases does not necessarily mean a cell being totally free of any further inorganic and/or organic compounds which were present in the culture medium.

Preferably purified shall mean herein that the cell according to the invention represents at least 95%, more preferably at least 96%, further more preferably at least 97%, even more preferably at least 98%, even further more preferably at least 99%, most preferably at least 99.5% of the total amount of the dry weight material comprising the cell being free from the culture medium.

The term “host cells have been (further) processed” shall mean herein that the host cells comprising a protein according to the invention have been treated with physical and/or chemical means before they are contacted in step c) of the method for production of an amine according to the invention with an amine acceptor molecule and an amine donor molecule, preferably they have been treated with physical means, more preferably they have been dried, further more preferably they have been freeze dried or spray dried, most preferably they have been spray dried.

Drying processes, in particular freeze dry and spray dry processes of cells are known for a person skilled in the art. Preferably the host cells comprising a protein according to the invention have been freeze dried or spray dried, most preferably they have been spray dried before they are contacted in step c) of the method for production of an amine according to the invention by the method described herein under “General Methods” item 9.

It is known to a person skilled in the art that proteins having the activity of an ω-TA are pyridoxal phosphate (PLP) dependent enzymes. In a preferred embodiment the protein is contacted in step c) of the method for producing an amine according to the invention with the amine acceptor molecule provided in step a) and the amine donor molecule provided in step b) in presence of PLP, more preferably PLP is present in an amount of between 0.05 g/l to 2.0 g/l, further more preferably in an amount of between 0.05 g/l to 1.5 g/l, even more preferably in an amount of between 0.05 g/l to 1.0 g/l, even further more preferably in an amount of between 0.075 g/l to 0.75 g/l, most preferably in an amount of between 0.1 g/l to 0.5 g/l.

Obtaining the amine in obligatory step d) in the method for the production of an amine can mean that the amine is present in the composition of step d) without any further purification of the amine produced or it can mean that the amine produced is further purified. Purification of the amine can be done by methods known to a person skilled in the art. Such methods for purification of the amine include but are not limited to methods involving precipitation, methods including chromatography, distillation, extraction, adsorption or filtration.

A preferred embodiment of the method for the production of an amine according to the invention is a method for production of a composition comprising an (S)-amine in enantiomeric excess over its (respective) (R)-amine comprising the steps of
a) providing an amine acceptor molecule;
b) providing an amine donor molecule;
c) contacting the amine acceptor molecule provided in step a) and the amine donor molecule provided in step b) with a protein according to the invention;
d) optionally, obtaining a composition comprising an (S)-amine in enantiomeric excess over its (respective) (R)-amine.

The term “enantiomer” as used herein has the meaning as commonly understood in the art of chemistry being a molecule which is one of two stereoisomers that are structural mirror images of each other that are non-superposable. The term “Enantiomer” is commonly also known as “optical isomer”.

The term “enantiomeric excess (commonly abbreviated by “ee”)” is commonly understood in the technical field of chemistry and used herein to specify the excess of one enantiomer in a composition over the respective other one defined as the absolute difference between the mole fractions of each enantiomer. Often enantiomeric excess is expressed in the art in percent enantiomeric excess. As an example, a composition comprising 70% of an (S)-enantiomer and 30% of an (R)-enantiomer has concerning the (S)-enantiomer an ee=40% (40% pure (S)-enantiomer+60% racemic (=30% (S)+30% (R)). Conclusively, racemic enantiomer mixtures have an ee=0%, pure (S)- or (R)-enantiomers have an ee=100%.

A preferred embodiment of the method according to the invention for production of a composition comprising an (S)-amine in enantiomeric excess is a method for production of an aliphatic (S)-amine (including but not limited to linear, branched or cyclic alkan amines, alken amines, alkyn amines) in enantiomeric excess or is a method for production of an aryl (S)-amine in enantiomeric excess or is a method for production of an (S)-amino acid in enantiomeric excess, more preferably a method for production of an (S)-α-amino acid in enantiomeric excess, further more preferably a method for production of a branched (S)-α-amino acid, an aromatic (S)-α-amino acid or an aromatic (S)-α-amino acid comprising substituted phenyl groups in enantiomeric excess, most preferably a method for production of the amino acids (S)-norvaline, (S)-leucine, (S)-phenylalanine or (S)-tyrosine in enantiomeric excess.

In respect to the ω-TA variants according to the invention comprising further amino acid modifications the method according to the invention for production of a composition comprising an (S)-amine in enantiomeric excess preferably is a method for production of a phosphorous comprising aliphatic (S)-amine (including but not limited to a phosphorous comprising linear, branched or cyclic alkan (S)-amine, alken (S)-amine, alkyn (S)-amine) in enantiomeric excess or is a method for production of a phosphorous comprising aryl (S)-amine in enantiomeric excess or is a method for production of a phosphorous comprising (S)-amino acid in enantiomeric excess, more preferably a method for production of a phosphorous comprising (S)-α-amino acid in enantiomeric excess, further more preferably a method for production of a phosphorous comprising branched (S)-α-amino acid, a phosphorous comprising aromatic (S)-α-amino acid or a phosphorous comprising aromatic (S)-α-amino acid comprising substituted phenyl groups in enantiomeric excess, even more preferably a method for production of a phosphorous comprising (S)-α-amino acid in enantiomeric excess, even further more preferably a method for production of an (S)-α-amino acid comprising a methyl substituted phosphorous in enantiomeric excess, most preferably a method for production of (S)-glufosinate in enantiomeric excess.

Another preferred embodiment of the method according to the invention for production of a composition comprising an (S)-amine in enantiomeric excess, is a method for production of a composition comprising an (S)-amine in an enantiomeric excess (ee) of at least 20%, more preferably of at least 40%, further more preferably of at least 60%, even more preferably of at least 80%, even further more preferably of at least 90%, particular preferably at least of 94%, most preferably of at least 96% or especially preferably of at least 98%.

What has been defined herein above in respect to preferred embodiments of amine acceptor molecules to be provided and preferred embodiments of the amounts to be provided in step a) and preferred embodiments of the amine donor molecule to be provided and preferred embodiments of the amounts to be provided in step b) of the method according to the invention for production of an amine is applicable accordingly to the amine acceptor molecule in step a) and the amine donor molecule in step b), respectively, in the method for production of a composition comprising an (S)-amine in enantiomeric excess over its (respective) (R)-amine. It is however self-evident, that in case that the amine donor molecules to be provided in step b) in the method for production of a composition comprising an (S)-amine in enantiomeric excess over its (respective) (R)-amine is a chiral molecule, at least an enantiomer mixture comprising an (S)-stereoisomer of the amine donor is provided, preferably a racemic mixture of an amine donor is provided. If available and feasible in terms of economic costs, the chiral amine donor can preferably be provided in a mixture where the (S)-stereoisomer is in enantiomeric excess, more preferably, the amine donor can be provided as a composition comprising the (S)-stereoisomer in high enantiomeric excess, in which case high enantiomeric excess means an enantiomeric excess of at least 30%, more preferably of at least 40%, further more preferably of at least 60%, even more preferably of at least 80%, even further more preferably of at least 90%, particular preferably at least of 94%, most preferably of at least 96% or especially preferably of at least 98%.

What has been defined herein above in respect to preferred embodiments of solutions, aqueous solutions, aqueous solutions comprising organic solvents, buffer systems, pH values and/or temperature, the form of the protein (crude cell extract, partially purified protein, purified protein, protein present as a component of a living or non-living host cell, (further) processed host cell, spray drying of host cell), the amount of protein and the presence and amount of PLP concerning step c) of the method according to the invention for production of an amine is applicable accordingly to step c) of the method for production of a composition comprising an (S)-amine in enantiomeric excess over its (respective) (R)-amine.

What has been defined herein above in respect to preferred embodiments of step d) of the method according to the invention for production of an amine is applicable accordingly to step d) of the method for production of a composition comprising an (S)-amine in enantiomeric excess over its (respective) (R)-amine.

In addition to what has been defined for step d) of the method according to the invention for production of an amine, preferably a composition comprising an (S)-amine in an enantiomeric excess of at least 40%, more preferably of at least 70%, further more preferably of at least 80%, even more preferably of at least 90%, even further more preferably of at least 95%, particular preferably at least of 97%, most preferably of at least 98% or especially preferably of at least 99% is obtained in step d) of the method for production of a composition comprising an (S)-amine in enantiomeric excess over its (respective) (R)-amine.

Proteins according to the invention can also be used in methods for decreasing or eliminating a stereoisomer from compositions comprising (R)- and (S)-amine stereoisomers. The reaction catalyzed by proteins according to the invention when decreasing or eliminating a stereoisomer from compositions comprising (R)- and (S)-amine isomers follows the general equation (Ia). Compared to the reaction for synthesis of amines (see equation (I)) amino donor and amino acceptors can be seen to be exchanged with each other in reactions decreasing or eliminating a stereoisomer from compositions comprising (R)- and (S)-amines (see equation (Ia)) The reaction according to equation (la) has the advantage that specific stereoisomers can be enriched in compositions comprising different stereoisomers or in other words, specific stereoisomers can be removed form a composition, sometimes also designated in the art as resolving an enantiomer mixture. Those methods are of particular importance in cases a compound is produced by way of chemical synthesis which commonly leads to a racemic mixture. Chemical synthesis of such a compound may be the desired production process in terms of process economy or other reasons. However, separation of chemically produced enantiomers may be difficult, expensive or even not possible. Proteins according to the invention can be used for selectively removing a stereoisomer from such chemically produced racemic mixtures.

A further embodiment of the invention therefore pertains a method for decreasing the amount an amine enantiomer in a composition comprising (R)-amines and (S)-amines, comprising the steps of
a) providing a composition comprising (R)-amine and (S)-amine enantiomers
b) providing an amine acceptor molecule;
c) contacting the composition provided in step a) and the amine acceptor provided in step b) with a protein according to the invention;
d) optionally, obtaining a composition in which the amount of an amine enantiomer is decreased compared to the amount present in the composition provided in step a).

In the method for decreasing the amount of an amine enantiomer in a composition comprising (R)-amines and (S)-amines it is not decisive how many structurally different (R)-amine and (S)-amine molecules are present in the composition provided in step a) of each of these methods, as long at least one (S)-amine and one (R)-amine molecule is present.

The composition comprising (R)- and (S)-amines provided in step a) of the method for decreasing the amount an amine enantiomer in a composition comprising (R)- and (S)-amines comprises at least one (R)-amine and at least one (S)-amine, wherein the at least one (R)-amine and the at least one (S)-amine can be stereoisomers of the same molecule or the at least one (R)-amine and the at least one (S)-amine can be stereoisomers from structurally different molecules.

A preferred embodiment of the a method for decreasing the amount an amine enantiomer in a composition comprising (R)-amines and (S)-amines is a method for decreasing the amount of an enantiomer of an aliphatic amine (including but not limited to linear, branched or cyclic alkan amines, alken amines, alkyn amines) or is a method for decreasing the amount of an enantiomer of an aryl amine or is a method for decreasing the amount of an enantiomer of an amino acid, more preferably a method for decreasing the amount of an enantiomer of an α-amino acid, further more preferably a method for decreasing the amount an of enantiomer of a branched α-amino acid, an enantiomer of an aromatic α-amino acid or an enantiomer of an aromatic α-amino acid comprising substituted phenyl groups, most preferably a method for decreasing the amount of an enantiomer of the amino acids selected from norvaline, leucine, phenylalanine or tyrosine.

In respect to the ω-TA variants comprising further amino acid modifications according to the invention the method according to the invention for decreasing the amount of an amine enantiomer in a composition comprising (R)- and (S)-amines preferably is a method for decreasing the amount of an enantiomer of a phosphorous comprising aliphatic amine (including but not limited to a phosphorous comprising linear, branched or cyclic alkan amine, alken amine, alkyn amine) or is a method for decreasing the amount of an enantiomer of a phosphorous comprising aryl amine or is a method for decreasing the amount of an enantiomer of a phosphorous comprising amino acid, more preferably a method for decreasing the amount of an enantiomer of a phosphorous comprising α-amino acid, further more preferably a method for decreasing the amount of an enantiomer of a phosphorous comprising branched α-amino acid, an enantiomer of a phosphorous comprising aromatic α-amino acid or an enantiomer of a phosphorous comprising aromatic α-amino acid comprising substituted phenyl groups, even more preferably a method for decreasing the amount of an enantiomer of a substituted phosphorous comprising α-amino acid, even further more preferably a method for decreasing the amount of an enantiomer of an α-amino acid comprising a methyl substituted phosphorous, most preferably a method for decreasing the amount of an enantiomer of glufosinate.

What has been defined herein above in respect to preferred embodiments of solutions, aqueous solutions, aqueous solutions comprising organic solvents, buffer systems, pH values and/or temperature, the form of the protein (crude cell extract, partially purified protein, purified protein, protein present as a component of a living or non-living host cell, (further) processed host cell, spray drying of host cell), the amount of protein and the presence and amount of PLP concerning step c) of the method according to the invention for production of an amine is applicable accordingly to step c) of the method for decreasing the amount an amine enantiomer in a composition comprising (R)-amines and (S)-amines.

What has been defined herein above in respect to preferred embodiments of step d) of the method according to the invention for production of an amine is applicable accordingly to step d) of the method for decreasing the amount an amine enantiomer in a composition comprising (R)-amines and (S)-amines.

Proteins according to the invention can in particular be used in methods for decreasing the amount of or substantially or nearly totally eliminating (S)-enantiomers from compositions comprising (R)-amine and (S)-amine stereoisomers, thereby producing compositions wherein an (R)-amine is present in enantiomeric excess. The respective reaction catalysed in methods for production of enantiomerically enriched or nearly enantiomerically pure amines by (S)-selective ω-TAs formally can be described by the general equation (II)

R¹—CH((S,R)—NH₂)—R²+R³—CO—R⁴→R¹—CO—R²+R³—CH((R)—NH₂)—R⁴

Many compounds having biological activity, like medicals, active compounds used in agronomy, supplementary food additives, feed additives etc. exist as enantiomers. In the vast majority of cases only one of the enantiomers shows the desired biological activity and the other one is inactive or often even shows undesired side effects. Today numerous compounds having biological activity and used as medicals, in agronomy, as supplementary food or feed additives (e.g. amino acids) can only be produced or can only be produced under economically feasible conditions by way of chemical synthesis with the disadvantage that those compounds are available only as racemic mixtures. Proteins according to the invention provide the advantage that the amount of (S)-amines can be partially, significantly or nearly totally removed from such racemic mixtures, with the effect that compositions are obtained comprising the biological active enantiomer or a precursor for use in a production process of biological active enantiomers in access or comprising the biological active enantiomer or a precursor thereof in a composition being nearly free of the inactive enantiomer. This reduces side effects in medicals, products used in agronomy or products comprising supplementary food or feed additives.

In a preferred embodiment the method for decreasing the amount an amine enantiomer in a composition comprising (R)-amine and (S)-amine enantiomers is a method for decreasing the amount of an (S)-amine enantiomer in a composition comprising (R)-amines and (S)-amines comprising the steps of
a) providing a composition comprising (R)-amines and (S)-amines;
b) providing an amine acceptor molecule;
c) contacting the composition provided in step a) and the amine acceptor molecule provided in step b) with a protein according to the invention;
d) optionally, obtaining a composition in which the amount of an (S)-amine enantiomer is decreased compared to the amount present in the composition provided in step a).

A preferred embodiment of the method for decreasing the amount of an (S)-amine enantiomer in a composition comprising (R)- and (S)-amine enantiomers is a method for decreasing the amount of an aliphatic (S)-amine (including but not limited to linear, branched or cyclic alkan (S)-amines, alken (S)-amines, alkyn (S)-amines) or is a method for decreasing the amount of an aryl (S)-amine or for decreasing the amount of an (S)-amino acid, more preferably a method for decreasing the amount of an (S)-α-amino acid, further more preferably a method for decreasing the amount of a branched (S)-α-amino acid, an aromatic (S)-α-amino acid or an aromatic (S)-α-amino acid comprising substituted phenyl groups, most preferably a method for decreasing the amount of the amino acids selected from (S)-norvaline, (S)-leucine, (S)-phenylalanine or (S)-tyrosine.

In respect to the ω-TA variants comprising further amino acid modifications according to the invention the method according to the invention for decreasing the amount of an (S)-amine enantiomer in a composition comprising (R)- and (S)-amines preferably is a method for decreasing the amount of a phosphorous comprising aliphatic (S)-amine (including but not limited to a phosphorous comprising linear, branched or cyclic alkan (S)-amine, alken (S)-amine, alkyn (S)-amine) or is a method for decreasing the amount of a phosphorous comprising aryl (S)-amine or is a method for decreasing the amount of a phosphorous comprising (S)-amino acid, more preferably a method for decreasing the amount of a phosphorous comprising (S)-α-amino acid, further more preferably a method for decreasing the amount of a phosphorous comprising branched (S)-α-amino acid, a phosphorous comprising aromatic (S)-α-amino acid or a phosphorous comprising aromatic (S)-α-amino acid comprising substituted phenyl groups, even more preferably a method for decreasing the amount of a substituted phosphorous comprising (S)-α-amino acid, even further more preferably a method for decreasing the amount of an (S)-α-amino acid comprising a methyl substituted phosphorous, most preferably a method for decreasing the amount of (S)-glufosinate.

Preferably the composition comprising (R)- and (S)-amines provided in step a) of each of the methods for decreasing the amount an amine enantiomer in a composition comprising (R)-amines and (S)-amines or the method for decreasing the amount of an (S)-amine enantiomer in a composition comprising (R)-amines and (S)-amines comprises (R)- and/or (S)-amines selected from the group of compounds selected from aliphatic (R)- and (S)-amines (including but not limited to linear, branched or cyclic alkan (R)- and (S)-amines, alken (R)- and (S)-amines, alkyn (R)- and (S)-amines) or aryl (R)- and (S)-amines or (R)- and (S)-amino acids, more preferably (R)- and (S)-α-amino acids, further more preferably branched (R)- and (S)-α-amino acids, aromatic (R)- and (S)-α-amino acids or aromatic (R)- and (S)-α-amino acids comprising substituted phenyl groups, most preferably the amino acids (R)- and (S)-norvaline, (R)- and (S)-leucine, (R)- and (S)-phenylalanine or (R)- and (S)-tyrosine.

In respect to the ω-TA variants according to the invention comprising further amino acid modifications preferably the composition comprising (R)- and (S)-amines provided in step a) of each of the methods for decreasing the amount an amine enantiomer in a composition comprising (R)-amines and (S)-amines or the method for decreasing the amount of an (S)-amine enantiomer in a composition comprising (R)-amines and (S)-amines comprises (R)- and/or (S)-amines from the group of compounds selected from phosphorous comprising aliphatic (R)- and (S)-amines (including but not limited to phosphorous comprising linear, branched or cyclic alkan (R)- and (S)-amines, alken (R)- and (S)-amines, alkyn (R)- and (S)-amines) or phosphorous comprising aryl (R)- and (S)-amines or phosphorous comprising (R)- and (S)-amino acids, more preferably phosphorous comprising (R)- and (S)-α-amino acids, further more preferably phosphorous comprising branched (R)- and (S)-α-amino acids, phosphorous comprising aromatic (R)- and (S)-α-amino acids or a phosphorous comprising aromatic (R)- and (S)-α-amino acids comprising substituted phenyl groups, even more preferably substituted phosphorous comprising (R)- and (S)-α-amino acids, even further more preferably (R)- and (S)-α-amino acids comprising a methyl substituted phosphorous, most preferably (R)- and (S)-glufosinate.

More preferably, the composition comprising (R)- and (S)-amines provided in step a) of each of the methods for decreasing the amount an amine enantiomer in a composition comprising (R)- and (S)-amines or the method for decreasing the amount of an (S)-amine enantiomer in a composition comprising (R)- and (S)-amines comprises (R)- and (S)-amines of the same molecule, more preferably it comprises (R)- and (S)-amines each representing an enantiomer of one single compound selected from the group of compounds consisting of aliphatic (R)- and (S)-amines (including but not limited to linear, branched or cyclic alkan (R)- and (S)-amines, alken (R)- and (S)-amines, alkyn (R)- and (S)-amines) or aryl (R)- and (S)-amines or (R)- and (S)-amino acids, more preferably (R)- and (S)-α-amino acids, further more preferably branched (R)- and (S)-α-amino acids, aromatic (R)- and (S)-α-amino acids or aromatic (R)- and (S)-α-amino acids comprising substituted phenyl groups, most preferably the amino acids (R)- and (S)-norvaline, (R)- and (S)-leucine, (R)- and (S)-phenylalanine or (R)- and (S)-tyrosine.

In respect to the ω-TA variants according to the invention comprising further amino acid modifications preferably, the composition comprising (R)- and (S)-amines provided in step a) of each of the methods for decreasing the amount an amine enantiomer in a composition comprising (R)- and (S)-amines or the method for decreasing the amount of an (S)-amine enantiomer in a composition comprising (R)- and (S)-amines comprises (R)- and (S)-amines of the same molecule, more preferably it comprises (R)- and (S)-amines each representing an enantiomer of one single compound selected from selected from the group of compounds consisting of phosphorous comprising aliphatic (R)- and (S)-amines (including but not limited to a phosphorous comprising linear, branched or cyclic alkan (R)- and (S)-amines, alken (R)- and (S)-amines, alkyn (R)- and (S)-amines) or a phosphorous comprising aryl (R)- and (S)-amines or a phosphorous comprising (R)- and (S)-amino acids, more preferably a phosphorous comprising (R)- and (S)-α-amino acids, further more preferably a phosphorous comprising branched (R)- and (S)-α-amino acids, a phosphorous comprising aromatic (R)- and (S)-α-amino acids or a phosphorous comprising aromatic (R)- and (S)-α-amino acids comprising substituted phenyl groups, even more preferably a substituted phosphorous comprising (R)- and (S)-α-amino acids, even further more preferably (R)- and (S)-α-amino acids comprising a methyl substituted phosphorous, most preferably (R)- and (S)-glufosinate.

Preferably the amine acceptor molecule provided in step b) of each of the methods for decreasing the amount an amine enantiomer in a composition comprising (R)-amines and (S)-amines or the method for decreasing the amount of an (S)-amine enantiomer in a composition comprising (R)-amines and (S)-amines is a molecule which structure corresponds to the structure described as amine donor molecule herein above to be provided in step b) of the method for the production of an amine as an amine donor apart from that the amine group of those molecules described as amine donor molecule herein above to be provided in step b) of the method for the production of an amine is replaced by a carbonyl group. As an example, replacing the amine group of isopropylamine described as am amine donor molecule to be provided in step b) of the method for the production of an amine by a carbonyl group leads to the corresponding amine acceptor molecule acetone to be used in step b) of each of the methods for decreasing the amount an amine enantiomer in a composition comprising (R)-amines and (S)-amines or the method for decreasing the amount of an (S)-amine enantiomer in a composition comprising (R)-amines and (S)-amines.

The most preferred amine acceptor molecule provided in step b) of each of the methods for decreasing the amount an amine enantiomer in a composition comprising (R)-amines and (S)-amines or the method for decreasing the amount of an (S)-amine enantiomer in a composition comprising (R)-amines and (S)-amines is acetone.

What has been defined herein above in respect to preferred embodiments of solutions, aqueous solutions, aqueous solutions comprising organic solvents, buffer systems, pH values and/or temperature, the form of the protein (crude cell extract, partially purified protein, purified protein, protein present as a component of a living or non-living host cell, (further) processed host cell, spray drying of host cell), the amount of protein and the presence and amount of PLP concerning step c) of the method according to the invention for production of an amine is applicable accordingly to step c) of the method for decreasing the amount of an (S)-amine enantiomer in a composition comprising (R)-amines and (S)-amines.

What has been defined herein above in respect to preferred embodiments of step d) of the method according to the invention for production of an amine is applicable accordingly to step d) of the method for decreasing the amount of an (S)-amine enantiomer in a composition comprising (R)-amines and (S)-amines.

A further embodiment of the inventions is the use of proteins according to the invention for production of an amine, preferably for production of an (S)-amine.

Use of proteins according to the invention for decreasing the amount of an amine, preferably the amount of an (S)-amine in an enantiomeric mixture is also an embodiment of the invention.

The use of nucleic acid molecules according to the invention for expressing a protein according to the invention in a host cell according to the invention is also an embodiment of the invention.

Another embodiment of the inventions concerns the use of nucleic acid molecules according to the invention, recombinant nucleic acid molecules according to the invention, plasmids according to the invention, or vectors according to the invention, for transforming or genetically modifying a host cell according to the invention or for production of a protein according to the invention.

Use of a host cell according to the invention for production of an amine or for decreasing the amount of an amine, preferably the amount of an (S)-amine in an enantiomeric mixture is also an embodiment of the invention.

Description of the Sequences

Throughout the application, nucleotide and amino acid abbreviations are used according to the following IUPAC codes:

TABLE 3

| IUPAC | |
|---|---|
| nucleotide code | Base |
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| — | gap |

For discrimination between amino acids and nucleotides, capitalized nucleotide code abbreviations given in above Table are written herein in lower case.

TABLE 4

| IUPAC | Three | |
|---|---|---|
| amino acid | letter | |
| code | code | Amino acid |
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Codon usage follows herein the so called “general genetic code” according to the following Table, wherein “t” is to be substituted by “u” in ribonucleic acid (RNA) sequences. “TLC” stands for Three Letter Code and “SLC” for Single Letter Code of amino acids.

TABLE 5

| | | | | Codons due to |
|---|---|---|---|---|
| | | | | degenerate genetic |
| Amino Acid | TLC | SLC | DNA codons | code |
| Alanine | Ala | A | gca | gcn |
| Alanine | Ala | A | gcc | gcn |
| Alanine | Ala | A | gcg | gcn |
| Alanine | Ala | A | gct | gcn |
| Arginine | Arg | R | aga | mgn |
| Arginine | Arg | R | agg | mgn |
| Arginine | Arg | R | cga | mgn |
| Arginine | Arg | R | cgc | mgn |
| Arginine | Arg | R | cgg | mgn |
| Arginine | Arg | R | cgt | mgn |
| Asparagine | Asn | N | aac | aay |
| Asparagine | Asn | N | aat | aay |
| Aspartic acid | Asp | D | gac | gay |
| Aspartic acid | Asp | D | gat | gay |
| Cysteine | Cys | C | tgc | tgy |
| Cysteine | Cys | C | tgt | tgy |
| Glutamic acid | Glu | E | gaa | gar |
| Glutamic acid | Glu | E | gag | gar |
| Glutamine | Gln | Q | caa | car |
| Glutamine | Gln | Q | cag | car |
| Glycine | Gly | G | gga | ggn |
| Glycine | Gly | G | ggc | ggn |
| Glycine | Gly | G | ggg | ggn |
| Glycine | Gly | G | ggt | ggn |
| Histidine | His | H | cac | cay |
| Histidine | His | H | cat | cay |
| Isoleucine | Ile | I | ata | ath |
| Isoleucine | Ile | I | atc | ath |
| Isoleucine | Ile | I | att | ath |
| Leucine | Leu | L | cta | ytn |
| Leucine | Leu | L | ctc | ytn |
| Leucine | Leu | L | ctg | ytn |
| Leucine | Leu | L | ctt | ytn |
| Leucine | Leu | L | tta | ytn |
| Leucine | Leu | L | ttg | ytn |
| Lysine | Lys | K | aaa | aar |
| Lysine | Lys | K | aag | aar |
| Methionine | Met | M | atg | atg |
| Phenylalanine | Phe | F | ttc | tty |
| Phenylalanine | Phe | F | ttt | tty |
| Proline | Pro | P | cca | ccn |
| Proline | Pro | P | ccc | ccn |
| Proline | Pro | P | ccg | ccn |
| Proline | Pro | P | cct | ccn |
| Serine | Ser | S | agc | wsn |
| Serine | Ser | S | agt | wsn |
| Serine | Ser | S | tca | wsn |
| Serine | Ser | S | tcc | wsn |
| Serine | Ser | S | tcg | wsn |
| Serine | Ser | S | tct | wsn |
| Threonine | Thr | T | aca | acn |
| Threonine | Thr | T | acc | acn |
| Threonine | Thr | T | acg | acn |
| Threonine | Thr | T | act | acn |
| Tryptophan | Thr | W | tgg | tgg |
| Tyrosine | Tyr | Y | tac | tay |
| Tyrosine | Tyr | Y | tat | tay |
| Valine | Val | V | gta | gtn |
| Valine | Val | V | gtc | gtn |
| Valine | Val | V | gtg | gtn |
| Valine | Val | V | gtt | gtn |
| Stop codons | Stop | Stop | taa | trr |
| Stop codons | Stop | Stop | tag | trr |
| Stop codons | Stop | Stop | tga | trr |

SEQ ID NO 1: Nucleic acid sequence encoding an omega-transaminase (ω-TA) from Bacillus megaterium obtained by back-translation of the amino acid sequence shown under SEQ ID NO 3, wherein the back-translation follows the principle of translation due to degeneracy of the general genetic code. Nucleotides at positions 1432 to 1449 encoding six His amino acids were inserted into the sequence from Bacillus megaterium before the stop codon located at positions 1450 to 1452.

SEQ ID NO 2: Nucleic acid sequence encoding an ω-TA from Bacillus megaterium having the amino acid sequence as shown under SEQ ID NO 3. Nucleotides at positions 1432 to 1449 encoding six His amino acids were inserted into the sequence from Bacillus megaterium before the stop codon located at positions 1450 to 1452.

SEQ ID NO 3: Amino acid sequence of an ω-TA from Bacillus megaterium derivable from GenPept (PDB) under accession No 5G09_A. The amino acid shown is encoded by the nucleic acid sequences as shown under SEQ ID NOs 1 and 2. The six His amino acids at positions 478 to 483 were inserted into the sequence from Bacillus megaterium by means of sequence modification.

SEQ ID NO 4: Nucleic acid sequence encoding an ω-TA from Arthrobacter sp. obtained by back-translation of the amino acid sequence shown under SEQ ID NO 6, wherein the back-translation follows the principle of translation due to degeneracy of the general genetic code.

SEQ ID NO 5: Nucleic acid sequence encoding an ω-TA from Arthrobacter sp. having the amino acid sequence as shown under SEQ ID NO 6. Nucleotides at positions 1438 to 1455 encoding six His amino acids were inserted into the sequence from Arthrobacter sp. before the stop codon located at positions 1456 to 1458.

SEQ ID NO 6: Amino acid sequence of an ω-TA from Arthrobacter sp. derivable from GenPept (PDB) under accession No 5G2P_A. The amino acid shown is encoded by nucleic acid sequences as shown under SEQ ID NOs 4 and 5. The six His amino acids at positions 480 to 485 were inserted into the sequence from Arthrobacter sp. by means of sequence modification.

SEQ ID NO 7: Nucleic acid sequence encoding an ω-TA from Bacillus sp. (soil 76801 D1 obtained by back-translation of the amino acid sequence shown under SEQ ID NO 9, wherein the back-translation follows the principle of translation due to degeneracy of the general genetic code.

SEQ ID NO 8: Nucleic acid sequence encoding an ω-TA from Bacillus sp. (soil 76801 D1 derivable from GenBank accession No. LMTA01000079.1.

SEQ ID NO 9: Amino acid sequence of an ω-TA from Bacillus sp. (soil 76801 D1 derivable from GenPept (PDB) under accession No. KRF52528.1. The amino shown is encoded by nucleic acid sequences as shown under SEQ ID NOs 7 and 8, described herein above.

SEQ ID NO 10: Nucleic acid sequence encoding a mutated ω-TA from Arthrobacter sp. obtained by back-translation of the amino acid sequence shown under SEQ ID NO 12, wherein the back-translation follows the principle of translation due to degeneracy of the general genetic code.

SEQ ID NO 11: Nucleic acid sequence encoding a mutated ω-TA variant from Arthrobacter sp. having the amino acid sequence as shown under SEQ ID NO 12. The sequence is derivable form SEQ ID NO 15 in WO 2006/063336 A2.

SEQ ID NO 12: Amino acid sequence of a mutated ω-TA from Arthrobacter sp. derivable from SEQ ID NO 16 in WO 2006/06336 A2. The amino acid shown is encoded by nucleic acid sequences as shown under SEQ ID NOs 11 and 12, described herein above.

SEQ ID NO 13: Nucleic acid sequence encoding a wild-type ω-TA from Arthrobacter sp. obtained by back-translation of the amino acid sequence shown under SEQ ID NO 15, wherein the back-translation follows the principle of translation due to degeneracy of the general genetic code.

SEQ ID NO 14: Nucleic acid sequence encoding a wild-type ω-TA from Arthrobacter sp. having the amino acid sequence as shown under SEQ ID NO 15. The sequence is derivable form SEQ ID NO 1 in WO 2006/063336 A2.

SEQ ID NO 15: Amino acid sequence of a wild-type ω-TA from Arthrobacter sp. derivable from SEQ ID NO 2 in WO 2006/06336 A2. The amino acid shown is encoded by nucleic acid sequences as shown under SEQ ID NOs 13 and 14, described herein above.

SEQ ID NO 16: Nucleic acid sequence encoding an improved ω-TA obtained by back-translation of the amino acid sequence shown under SEQ ID NO 18, wherein the back-translation follows the principle of translation due to degeneracy of the general genetic code.

SEQ ID NO 17: Nucleic acid sequence encoding an improved ω-TA having the amino acid sequence as shown under SEQ ID NO 18.

SEQ ID NO 18: Amino acid sequence of an improved ω-TA, wherein improvements are obtained by amino acid substitutions in comparison to the amino acid sequences from Bacillus megaterium shown under SEQ ID NOs 3 and 9 and in comparison to the amino acid sequences from Arthrobacter sp. shown under SEQ ID NO 6, 12 and 15.

SEQ ID NO 19: Nucleic acid coding sequence of the D-amino acid oxidase (DAO1) gene from Rhodotorula toruloides (synonym: Rhodotorula gracilis).

SEQ ID NO 20: Amino acid sequence of the protein having activity of a D-amino acid oxidase (DAO1) obtained from the coding sequence shown under SEQ ID NO 19.

SEQ ID NO 21: Nucleic acid coding sequence of a variant of the D-amino acid oxidase (DAO1) gene from Rhodotorula toruloides comprising compared to the nucleic acid sequence from Rhodotorula toruloides nucleotide substitutions (replacements) in the codons identified by the nucleotides at positions 160-162 and the codons identified by the nucleotides at positions 172-174 and the codons identified by the nucleotides at positions 637-639.

SEQ ID NO 22: Amino acid sequence of the protein having activity of a D-amino acid oxidase obtained from the coding sequence shown under SEQ ID NO 21. The amino acid sequence comprises amino acid substitutions (replacements) compared to the nucleic acid sequence from Rhodotorula toruloides at positions 54, 58 and 213, compared to the amino acid sequence shown under SEQ ID NO 21 and therefore is an amino acid sequence of a DAAO variant (mutant).

SEQ ID NO 23: Nucleic acid coding sequence of a catalase gene from Listeria seeligeri.

SEQ ID NO 24: Amino acid sequence of the protein having activity of a catalse obtained from the coding sequence shown under SEQ ID NO 23.

SEQ ID NO 25: Nucleic acid sequence encoding the protein having the amino acid sequence shown under SEQ ID NO 24 with the activity of a catalase.

SEQ ID NO 26: Nucleic acid sequence of the genetic element designated “lac operator” in FIG. 1.

SEQ ID NO 27: Nucleic acid sequence of the genetic element designated “Trc promotor” in FIG. 1.

SEQ ID NO 28: Nucleic acid sequence of the genetic element designated “rrnB” in FIG. 1.

SEQ ID NO 29: Nucleic acid sequence of the genetic element designated “cistron” in FIG. 1.

SEQ ID NO 30: Nucleic acid sequence of the genetic element designated “rrnB terminator” in FIG. 1.

### DESCRIPTION OF THE FIGURES

FIG. 1: Plasmid map showing the genetic elements used for the expression of proteins having the activity of a DAAO, ω-TA and catalase from a single operon as tri-cistronic RNA. Explanations to abbreviations of regulatory genetic elements involved in transcription and translation of the tri-cistronic RNA:

lac operator: Ullmann, 2001, Encyclopedia of Life Sciences, John Wiley & Sons, Ltd, ISBN: 9780470015902; Ullmann, 2009, Encyclopedia of Life Sciences (ELS), John Wiley & Sons, Ltd: Chichester. DOI: 10.1002/9780470015902.a0000849.pub2; consisting of the nucleic acid sequence shown under SEQ ID NO 26.

Trc promotor: Synthetic promoter derived from the E. coli trp and lacUV5 promoters (Brosius et al., 1985, J Biol Chem 260, 3539-3541); consisting of the nucleic acid sequence shown under SEQ ID NO 27.

rrnB: RhoI-independent transcription termination signal (Pfeiffer & Hartmann, 1997, J Mol Biol. 265(4) 385-393; Orosz et al., 1991, Eur J Biochem. 201(3), 653-659); consisting of the nucleic acid sequence shown under SEQ ID NO 28.

t7 enhancer: Transcription enhancing sequence from the t7 gene. (Sequence used: ttaacttta).

RBS1: Ribosome binding site (Sequence: gaggt).

cistron: Transcription termination sequence; consisting of the nucleic acid sequence shown under SEQ ID NO 29.

RBS2: Ribosome binding site (Sequence used: aaggag).

boxA: Transcriptional anti-termination sequence (Sequence used: tgctctttaacaa).

cistron: Synthetic cistron consisting of the nucleic acid sequence shown under SEQ ID NO 29.

rrnB terminator: Transcription termination signal; consisting of the nucleic acid sequence shown under SEQ ID NO 30.

T2 terminator: Translation termination signal (Orosz et al., 1991, Eur J Biochem. 201(3), 653-659).

FIG. 2: Presents the production of (S)-norvaline by amination of 2-oxovaleric acid ctalysed by wild-type ω-TA proteins from Arthrobacter sp. having the amino acid sequence shown under SEQ ID NO 6 or from Bacillus megaterium having the amino acid sequence shown under SEQ ID NO 3 compared to ω-TA variants having the amino acid sequence shown under SEQ ID NO 18.

FIG. 3: Presents the production of (S)-leucine by amination of 4-methyl-2-oxo-valeric acid ctalysed by wild-type ω-TA proteins from Arthrobacter sp. having the amino acid sequence shown under SEQ ID NO 6 or from Bacillus megaterium having the amino acid sequence shown under SEQ ID NO 3 compared to ω-TA variants having the amino acid sequence shown under SEQ ID NO 18.

FIG. 4: Presents the production of (S)-phenylalnine by amination of phenylpyruvic acid ctalysed by wild-type ω-TA proteins from Arthrobacter sp. having the amino acid sequence shown under SEQ ID NO 6 or from Bacillus megaterium having the amino acid sequence shown under SEQ ID NO 3 compared to ω-TA variants having the amino acid sequence shown under SEQ ID NO 18.

FIG. 5: Presents the production of (S)-tyrosine by amination of p-hydroxyphenylpyruvic acid ctalysed by wild-type ω-TA proteins from Arthrobacter sp. having the amino acid sequence shown under SEQ ID NO 6 or from Bacillus megaterium having the amino acid sequence shown under SEQ ID NO 3 compared to ω-TA variants having the amino acid sequence shown under SEQ ID NO 18.

### GENERAL METHODS

1. Production of ω-TA Variants and ω-TA Variants having Further Amino Acid Modifications

Known nucleotide sequences described herein encoding proteins having the activity of an ω-TA described herein were synthesized by the service provider by Eurofins Genomics GmbH (Eurofins Genomics GmbH, Anzinger Str. 7a, 85560 Ebersberg, Germany).

Into the nucleic acid sequences shown under SEQ ID Nos 2, 5, 8, 11, 14 nucleotide substitutions (replacements) were introduced. The replacement can be effected in the nucleic acid sequences which encode the reference polypeptide by any means which is appropriate for replacing nucleotides in nucleic acid sequences. Those methods are widely described in the literature and well known to the skilled person in the respective sequence. Several molecular biological methods can be used to achieve respective nucleotide replacements. A useful method for preparing a mutated nucleic acid sequence according to the invention and the corresponding protein comprises carrying out site-directed mutagenesis on codons encoding one or more amino acids which are selected in advance, thereby changing the selected codons in a way that they code for different amino acids. The methods for obtaining these site-directed mutations are well known to the skilled person and widely described in the literature (in particular: Directed Mutagenesis: A Practical Approach, 1991, Edited by M. J. McPHERSON, IRL PRESS), or are methods for which it is possible to employ commercial kits (for example the QUIKCHANGE™ lightening mutagenesis kit from Qiagen or Stratagene). After site-directed mutagenesis, nucleic acids were transformed into the Escherichia coli starin MG1655. The cells which contain a mutated polypeptide with advantageous biotransformation yields were selected by using an appropriate screening method. Appropriate screening methods are described herein under “General Methods”, items 4 and 7. Mutated nucleic acid sequences coding for improved polypeptides were sequence verified. The methods for sequence verification are well known to the skilled person and widely described in the literature (for example Sambrook and Russell (2012) Molecular Cloning: A Laboratory Manual (Cold Spring Harbor Laboratory Press, Cold Spring Harbor, N.Y.)).

2. Expression Vectors/Host Cells for ω-TA Variants

Nucleic acid sequences encoding wild-type ω-TAs (SEQ ID Nos 2, 5, 8, 14) or known ω-TAs comprising mutations (SEQ ID NO 11) or ω-TA variants as described herein were cloned into the commercial pET22B vector (Merck KGaA, Frankfurter Str 250, 64293 Darmstadt, Germany) and expressed in Escherichia coli strain BL21 DE3 cells.

3. Expression of ω-TA Variants

A pre-culture of Escherichia coli strain BL21 DE3 comprising the pET22B vector into which the respective nucleic acid sequence encoding ω-TA variants was introduced were grown in flasks containing 20 ml LB-Medium supplemented with carbenicillin at 37° C. on a rotary shaker at 180 rpm overnight. Expression of the coTA proteins was performed by transferring the pre-culture into flasks containing 250 ml LB-Medium supplemented with carbenicillin. Expression of the ω-TA proteins was induced by the addition of 0.5 mM IPTG (final concentration) after an OD (optical density) of between 0.6-0.8 was reached by growth at 37° C. on a rotary shaker at 180 rpm. The induced cell culture was incubated at 20° C. for 20 h at 180 rpm shaking. Purification of enzymes was performed using the Ni-NTA Fast Start Kit of Qiagen (Qiagen GmbH, Qiagen Strasse 1, 40724 Hilden) according to the manufacturers protocol.

4. Activity Test for ωTA Variants in Presence of Amine Acceptors and Amine Donors

To 40 μl triethanolamine buffer (200 mM solution in deionized water, pH=9,0), 10 μl pyridoxal phosphate (10 mM solution in deionized water) and 10 μl of the amino donor (2 M solution in deionized water, adjusted to pH=9,0 by addition of aqueous HCl) was added at room temperature. Subsequently, 20 μl of the amino acceptor (100 mM solution in deionized water) was added (if the amino acceptor is not soluble in water, proportional DMSO is added). Finally, 20 μl of the transaminase enzyme (1,5 mg/ml) was added at room temperature and the mixture was incubated at 40° C. on a rotary shaker at 800 rpm for 6-7 h. The transamination reaction was monitored by HPLC-analysis of aliquots taken at various time intervals during the reaction.

5. Expression Vectors/Host Cells Used for ω-TA Variants having Further Amino Acid Modifications

The activity test for ω-TA variants having further amino acid modifications was performed by using a method comprising two reaction steps.

The first reaction step (step 1) produces an amine acceptor for ω-TAs. The step is catalyzed by a D-amino acid oxidase (DAAO or DAO, EC 1.4.3.3). DAAOs are flavin adenine dinucleotide (FAD)-containing flavoproteins that catalyse the oxidative deamination of D-amino acids with oxygen to generate the corresponding 2-oxo acids along with hydrogen peroxide and ammonia according to the following general equation (III):

α-D-amino acid+H₂O+O₂→α-2-oxo carboxylic acid+NH₃+H₂O₂

The protein having the activity of a DAAO used for the production of α-2-oxo carboxylic acids in the first reaction step was a DAAO variant of the DAO1 protein from Rhodosporidium toruloides. The coding nucleic acid sequence of the wild-type DAO1 protein from Rhodosporidium toruloides is derivable from GenBank acc. No. U6006.1 (shown under SEQ ID NO 19) and the corresponding amino acid sequence encoded by the nucleic acid sequence shown under SEQ ID NO 19 is derivable from UniProt acc. No. P80324 (shown under SEQ ID NO 20). The DAAO variant used herein is disclosed in WO 201 7/1 51 573 as Mutant Ac305 (page 36, Table 1). Compared to SEQ ID NO 20 Mutant Ac305 comprises amino acid substations (replacements) at positions 54 and 58 and 213. In Mutant Ac305 the amino acid N at position 54 in SEQ ID NO 20 is substituted (replaced) by C and the amino acid F at position 58 in SEQ ID NO 20 is substituted (replaced) by H and the amino acid M at position 213 in SEQ ID NO 20 is substituted (replaced) by S. The amino acid sequence of Mutant Ac305 is shown under SEQ ID NO 22. A respective nucleic acid sequence encoding the protein having the amino acid sequence shown under SEQ ID NO 22 is shown under SEQ ID NO 21. The reaction of step 1 was catalyzed by a protein having the activity of a DAAO having the amino acid sequence shown under SEQ ID NO 22.

In a second reaction step (step 2) the α-2-oxo carboxylic acid produced by a protein having the activity of a DAAO) in step 1 is converted in presence of an amine donor by a protein having ω-TA activity into an amino acid according the general equation (I).

As becomes clear from description of step 1 by general equation (III), conversion of D-amino acids into keto acids catalysed by proteins having the activity of a DAAO generates hydrogen peroxide (H₂O2). Removal of H₂O₂ may be desired, but is not necessarily needed under every circumstance. Removal of H₂O₂ was accomplished in connection with the present invention by adding a protein having the activity of a catalase.

Proteins having the activity of a catalase (EC 1.11.1.6; hydrogen-peroxide:hydrogen-peroxide oxidoreductase) are known in the art and catalyze the conversion of hydrogen peroxide (H₂O₂) into water (H₂O) and oxygen (O₂) according to following general equation (IV):

2H₂O₂>O₂+2H₂O

The amino acid sequence of a protein having the activity of a catalase from Listeria seeligeri used for removal of H₂O₂ is shown under SEQ ID NO 24 and derivable under GenePept accession no. WP_012986600.1. SEQ ID NO 23 (derivable under GenBank accession no. NC_013891.1) shows the nucleic acid coding sequence from Listeria seeligeri for the catalase protein having the amino acid sequence as shown under SEQ ID NO 24. SEQ ID NO 25 is a nucleic acid sequence also encoding the catalase protein having the amino acid sequence shown under SEQ ID NO 24. Compared to the nucleic acid sequence shown under SEQ ID VNO 23 codons of the nucleic acid sequence shown under SEQ ID NO 25 have been adapted to the codon usage of Escherichia coli.

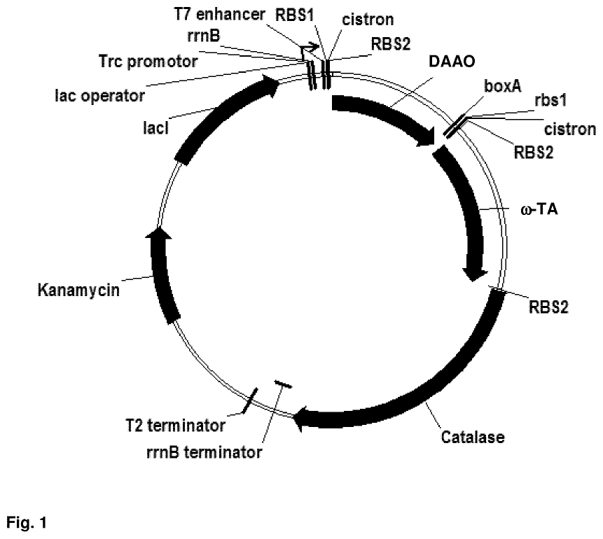

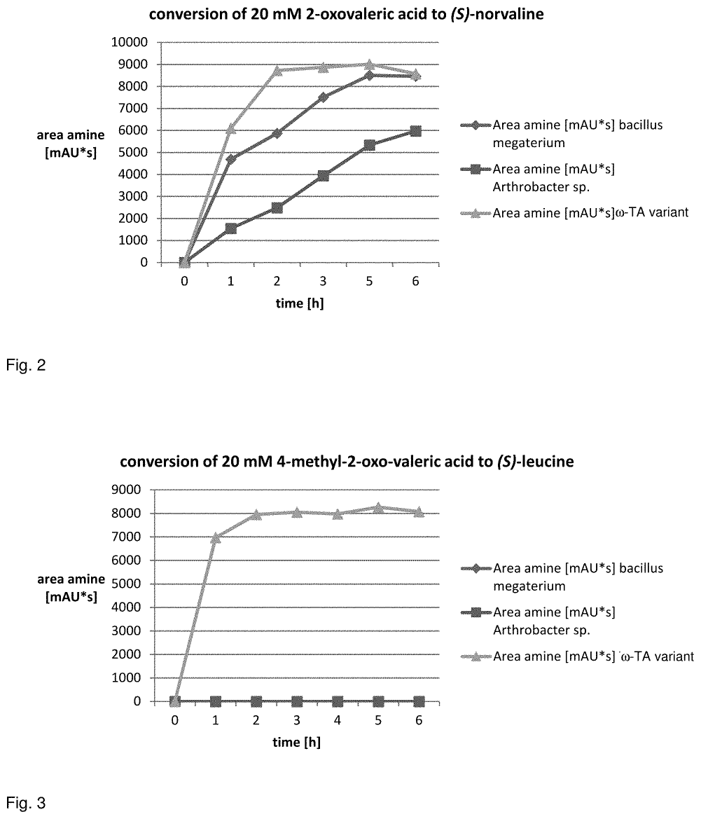

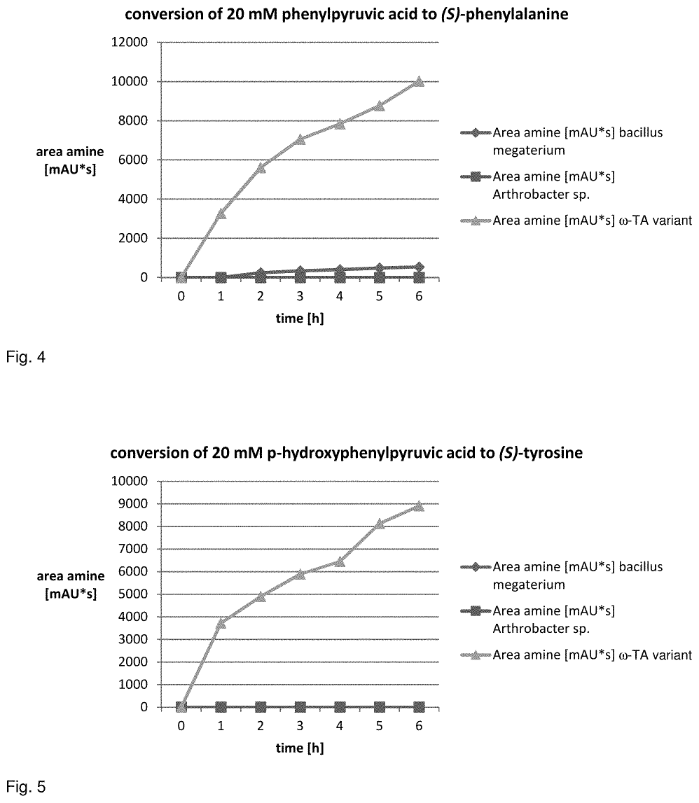

For producing proteins having the activity of a DAAO, proteins having the activity of an ω-TA and proteins having the activity of a catalase, nucleic acid sequences encoding the respective three proteins were cloned into an E. coli expression vector in a manner that all three proteins were transcribed from a single operon as tri-cistronic RNA from the trc-promoter (a hybrid promoter composed of sequences originating from the trp- and the lacUV5-promoter). The order of the genes in respect to transcription from the promoter was DAAO (SEQ ID NO 21)→nucleic acid molecules encoding ω-TA variants comprising further amino acid modifications (as described herein above)→catalase (SEQ ID NO 25). SEQ ID NO 21 was translationally fused at its 5′-end with a nucleic acid sequence encoding the amino acid sequence M A R I R L. The expression vector used is based on pSE420 (description and sequences derivable from: Addgene, 75 Sidney St, Suite 550A, Cambridge, Mass. 02139; https://www.addgene.org/vector-database/4064/ or from Thermo Fisher Scientific (Invitrogen), Thermo Fisher Scientific Inc. 168 Third Avenue, Waltham, Mass. 02451 USA, https://www.thermofisher.com/search/results?query=pSE420&focusarea). Genetic elements were introduced into a modified pSE420 vector by means of commonly known methods. The relevant genetic elements present in the expression vector used are shown in FIG. 1. For expression of the three enzymes he expression vector was transferred Escherichia coli strain MG1655 cells.

6. Expression of ω-TA Variants Comprising Further Amino Acid Modifications

ω-TA variants comprising further amino acid modifications were cloned into the tri-cistronic expression vector described above under “General Methods”, item 5 and expressed in Escherichia coli strain MG1655 cells. For this, a 20 ml pre-culture in LB-Medium supplemented with kanamycin was grown overnight in a shake flask at 37° C. on a rotary shaker at 180 rpm. Expression of the ωTA proteins was performed by transferring the pre-culture into flasks containing 200 ml LB-Medium supplemented with kanamycin. Expression of the ωTA proteins was induced after an OD of between 0.6-0.8 was reached by the addition of 1 mM IPTG (final concentration). The induced cell culture was incubated at 20° C. for 20 h at 180 rpm shaking. For harvest, the cell culture was centrifuged at 8000 g at 4° C. for 15 minutes and the obtained cell pellet was stored at −80° C. until freeze drying or spray drying.

7. Activity Test of ω-TA Variants Having Further Amino Acid Modifications

In a one litre temperature-adjustable glass double jacketed reactor equipped with a mechanical stirrer, an O₂-gas inlet tube and a pH-controlled dosing unit, 268 ml of an aqueous 50 w % racemic (R,S)-glufosinate ammonium solution (corresponds to 160,8 g racemic glufosinate ammonium) was added. Via the pH-controlled dosing unit an aqueous 2 M iso-propylamine solution was added under mechanical stirring (250 rpm) until a pH=9,0 was reached. The pH is kept constant during the entire reaction time by controlled addition of the aqueous 2 M iso-propylamine solution. The reactor is heated to 35° C. internal temperature.

In a beaker, 8 g of spray-dried Escherichia coli strain MG1655 cells containing the expression vector described in FIG. 1, expressing wild-type ω-TA proteins and ω-TA variants having further amino acid modifications, 200 mg pyridoxal phosphate, 2 ml polypropylene glycole (P 2000) and 138 ml deionized water were mixed. This mixture was added to the glass reactor under stirring (250 rpm) at 35° C. Via the O₂-gas inlet tube, oxygen gas was bubbled through the reaction mixture at a flow rate ofr 0,1 l/min. The mixture was stirred for 24 h and the reaction progress was monitored via HPLC-analysis of aliquots taken at various time intervals during the reaction. Afterwards, the oxygen gas feed as well as the iso-propylamine feed was stopped and the reaction mixture was denatured at 90° C. for 30 min under stirring (250 rpm). The residual mixture was cooled down to room temperature.

8. Detection of Amines Produced by ω-TAs

A) Analysis of Transamination Products (S)-norvaline, (S)-leucine, (S)-tyrosine and (S)-glufosinate Ammonium

The course of the transamination reaction was monitored via HPLC analysis. The HPLC methodology used in this work is based on the publication of Davankov et al. (1980, Chromatographia 13(11), 677-685).

Specifically, the following HPLC parameters were used:

Column: Phenomenex Chirex 3126 (D)—penicillamine 150*4,6 mm (Cat.: 00F-3126-E0)

Flow rate: 1 ml/min

Eluents: A) deionized water+0,5 g/L CuSO4 (v/v)

B) methanol

A:B=90:10 (isocratic)

Detector: DAD 230 nm

Oven: 30° C.

Runtime: 15 min

B) Analysis of Transamination Product L-Phenylalanine:

The course of the transamination reaction was monitored via HPLC analysis. Specifically, the following HPLC parameters were used:

Column: Phenomenex Prodigy 3 μm ODS-3 100A 100*4 mm (Cat.: 00D-4222-D0)

Flow rate: 2 ml/min

Eluents: A) acetonitrile

B) deionized water

Gradient from A:B=5:95 to A:B=95:5 within 7 min

Detector: VWD1 A, 210 nm

Oven: 40° C.

Run time: 9 min

9. Spray Drying of Cells

Spray drying experiments have been performed in a laboratory (Lab scale) spray dryer with a maximum temperature input of 220° C. The dryer uses either compressed air or nitrogen with 200-800 l/h (litre/hour) under 5-8 bar. The maximum airflow can be reached with 35 m3/h (meter3/hour).

In order to dry bacterial cell mass from either flask grown culture or fermented material (i.e. 1 litre total volume) the broth has been ten times (10×) concentrated by centrifugation and re-suspended to a final volume of 100 ml in culture supernatant obtained after cemntrifugation.

The obtained concentrate needs to be suitable for pumping and should be constantly mixed by a magnetic stirrer. The liquid was applied to a 0.7 mm nozzle using an airflow of 500 l/h with the aspirator set to 100%. The typical product flow was 10 ml/min. and the applied temperatures averaged for the inlet −145° C. and the outlet 85° C. The subsequent dried biomass has been 5 weighed and used in g/l scale for the biotransformation experiments.

### EXAMPLES

1. Conversion of 2-Oxovaleric Acid to (S)-Norvaline

Wild-type ω-TA proteins from Arthrobacter sp. having the amino acid sequence shown under SEQ ID NO 6 or from Bacillus megaterium having the amino acid sequence shown under SEQ ID NO 3 or ω-TA variants having the amino acid sequence shown under SEQ ID NO 18 were expressed and purified as described under “General Methods”, item 3.

To 25 μl triethanolamine buffer in deionized water (200 mM solution in deionized water, pH=9.0), 10 μl of pyridoxal phosphate (PLP) in deionized water (10 mM solution in deionized water) and 10 μl of iso-propylamine in deionized water (2 M solution in deionized water, adjusted to pH=9.0 by addition of aqueous HCl) was added at room temperature. Subsequently, 20 μl of 2-oxovaleric acid (100 mM solution in deionized water) was added. Finally, 35 μl of a solution comprising 1.5 mg/ml of the respective ω-TA protein was added at room temperature and the mixture was incubated at 40° C. on a rotary shaker at 800 rpm for 6 h. The transamination reaction was monitored by HPLC-analysis of aliquots taken at different time intervals during the reaction as described under “General Methods” item 8.

Table 6 presents the results obtained for an ω-TA variant having the amino acid sequence shown under SEQ ID NO 18 compared to those of wild-type proteins from Arthrobacter sp. having the amino acid sequence shown under SEQ ID NO 6 and Bacillus megaterium having the amino acid sequence shown under SEQ ID NO 3. The results are also shown in FIG. 2.

TABLE 6
Area amine [mAU * s]

| time | Area amine [mAU * s] Bacillus | Arthrobacter sp. (SEQ ID | Area amine [mAU * s] ω-TA |
|---|---|---|---|
| [h] | megaterium (SEQ ID NO 3) | NO 6) | variant (SEQ ID NO 18) |
| 0 | 0 | 0 | 0 |
| 1 | 4691 | 1551 | 6096 |
| 2 | 5871 | 2486 | 8727 |
| 3 | 7508 | 3946 | 8868 |
| 5 | 8500 | 5333 | 9014 |
| 6 | 8463 | 5973 | 8573 |

Description of Table 6:

“time” measured in hours (h) indicates the time elapsed since the reaction was started.

“mAU*s” is the abbreviation for milli (m) absorbance (A) units (U) multiplied (*) with seconds (s); a standard unit describing the area under a peak in a HPLC chromatogram. The higher the area under a peak, the higher is the amount of the respective product.

It is derivable form Table 6 and FIG. 2, that in the reaction catalyzed by the ω-TA variant production of (S)-norvaline from 2-oxovaleric acid proceeds faster than in the reactions catalyzed by wild-type proteins from Arthrobacter sp. and Bacillus megaterium. In addition, the maximum amount of (S)-norvaline produced during the reaction is reached significantly earlier in the reaction catalyzed by the ω-TA variant compared to reactions catalyzed by wild-type proteins from Arthrobacter sp. and Bacillus megaterium.

2. Conversion of 4-methyl-2-oxo-valeric Acid to (S)-Leucine

Wild-type ω-TA proteins from Arthrobacter sp. having the amino acid sequence shown under SEQ ID NO 3 or from Bacillus megaterium having the amino acid sequence shown under SEQ ID NO 6 or ω-TA variants having the amino acid sequence shown under SEQ ID NO 18 were expressed and purified as described under “General Methods”, item 3.

To 40 μl triethanolamine buffer (200 mM solution in deionized water, pH=9,0), 10 μl pyridoxal phosphate (10 mM solution in deionized water) and 10 μl of iso-propylamine (2 M solution in deionized water, adjusted to pH=9.0 by addition of aqueous HCl) was added at room temperature. Subsequently, 20 μl of 4-methyl-2-oxo-valeric acid (100 mM solution in deionized water) was added. Finally, 20 μl of a solution comprising 1.5 mg/ml of the respective ω-TA protein was added at room temperature and the mixture was incubated at 40° C. on a rotary shaker at 800 rpm for 6 h. The transamination reaction was monitored by HPLC-analysis of aliquots taken at different time intervals during the reaction as described under “General Methods” item 8.

Table 7 presents the results obtained for an ω-TA variant having the amino acid sequence shown under SEQ ID NO 18 compared to those of wild-type proteins from Arthrobacter sp. having the amino acid sequence shown under SEQ ID NO 3 and Bacillus megaterium having the amino acid sequence shown under SEQ ID NO 6. The results are also shown in FIG. 3.

TABLE 7
Area amine [mAU * s]

| time | Area amine [mAU * s] Bacillus | Arthrobacter sp. (SEQ ID | Area amine [mAU * s] ω-TA |
|---|---|---|---|
| [h] | megaterium (SEQ ID NO 3) | NO 6) | variant (SEQ ID NO 18) |
| 0 | 0 | 0 | 0 |
| 1 | 0 | 0 | 6971 |
| 2 | 0 | 0 | 7954 |
| 3 | 0 | 0 | 8052 |
| 4 | 0 | 0 | 7975 |
| 5 | 0 | 0 | 8267 |
| 6 | 0 | 0 | 8077 |

Description of Table 7: See description of Table 6

It is derivable from Table 7 and FIG. 3 that the wild-type enzymes from Arthrobacter sp. and Bacillus megaterium do not produce (S)-leucine by amination of 4-methyl-2-oxo-valeric acid, whereas the ω-TA variant does produce (S)-leucine quite efficiently.

3. Conversion of Phenylpyruvic Acid to (S)-Phenylalanine

Wild-type ω-TA proteins from Arthrobacter sp. having the amino acid sequence shown under SEQ ID NO 3 or from Bacillus megaterium having the amino acid sequence shown under SEQ ID NO 6 or ω-TA variants having the amino acid sequence shown under SEQ ID NO 18 were expressed and purified as described under “General Methods”, item 3.

To 40 μl triethanolamine buffer (200 mM solution in deionized water, pH=9.0), 10 μl pyridoxal phosphate (10 mM solution in deionized water) and 10 μl of iso-propylamine (2 M solution in deionized water, adjusted to pH=9.0 by addition of aqueous HCl) was added at room temperature. Subsequently, 20 μl of phenylpyruvic acid in DMSO/deionized water in a ratio 1:1 (100 mM phenylpyruvic acid solution) was added. Finally, 20 μl of a solution comprising 1.5 mg/ml of the respective ω-TA protein was added at room temperature and the mixture was incubated at 40° C. on a rotary shaker at 800 rpm for 6 h. The transamination reaction was monitored by HPLC-analysis of aliquots taken at different time intervals during the reaction as described under “General Methods” item 8.

Table 8 presents the results obtained for an ω-TA variant having the amino acid sequence shown under SEQ ID NO 18 compared to those of wild-type proteins from Arthrobacter sp. having the amino acid sequence shown under SEQ ID NO 3 and Bacillus megaterium having the amino acid sequence shown under SEQ ID NO 6. The results are also shown in FIG. 4.

TABLE 8
Area amine [mAU * s]

| time | Area amine [mAU * s] Bacillus | Arthrobacter sp. (SEQ ID | Area amine [mAU * s] ω-TA |
|---|---|---|---|
| [h] | megaterium SEQ ID NO 6) | NO 6) | variant (SEQ ID NO 18) |
| 0 | 0 | 0 | 0 |
| 1 | 0 | 0 | 3271 |
| 2 | 245 | 0 | 5613 |
| 3 | 338 | 0 | 7056 |
| 4 | 403 | 0 | 7845 |
| 5 | 481 | 0 | 8772 |
| 6 | 540 | 0 | 10022 |

Description of Table 8: See description of Table 6

It is derivable from Table 8 and FIG. 4 that the wild-type enzyme from Arthrobacter sp. does not produce (S)-phenylalanine from phenylpyruvic acid, the wild-type enzyme from Bacillus megaterium produces (S)-phenylalanine very slowly and in low amounts compared to the amount of (S)-phenylalanine produced by the ω-TA variant.

4. Conversion of p-hydroxyphenylpyruvic Acid to (S)-Tyrosine:

Wild-type ω-TA proteins from Arthrobacter sp. having the amino acid sequence shown under SEQ ID NO 3 or from Bacillus megaterium having the amino acid sequence shown under SEQ ID NO 6 or ω-TA variants having the amino acid sequence shown under SEQ ID NO 18 were expressed and purified as described under “General Methods”, item 3.

To 40 μl triethanolamine buffer (200 mM solution in deionized water, pH=9,0), 10 μl pyridoxal phosphate (10 mM solution in deionized water) and 10 μl of iso-propylamine (2 M solution in deionized water, adjusted to pH=9.0 by addition of aqueous HCl) was added at room temperature. Subsequently, 20 μl of p-hydroxyphenylpyruvic acid in DMSO/deionized water in a ratio 1:1 (100 mM p-hydroxyphenylpyruvic acid solution) was added. Finally, 20 μl of a solution comprising 1.5 mg/ml of the respective ω-TA protein was added at room temperature and the mixture was incubated at 40° C. on a rotary shaker at 800 rpm for 6 h. The transamination reaction was monitored by HPLC-analysis of aliquots taken at different time intervals during the reaction as described under “General Methods” item 8.

Table 9 presents the results obtained for an ω-TA variant having the amino acid sequence shown under SEQ ID NO 18 compared to those of wild-type proteins from Arthrobacter sp. having the amino acid sequence shown under SEQ ID NO 3 and Bacillus megaterium having the amino acid sequence shown under SEQ ID NO 6. The results are also shown in FIG. 5.

TABLE 9
Area amine [mAU * s]

| time | Area amine [mAU * s] Bacillus | Arthrobacter sp. (SEQ ID | Area amine [mAU * s] ω-TA |
|---|---|---|---|
| [h] | megaterium (SEQ ID NO 3) | NO 6) | variant (SEQ ID NO 18) |
| 0 | 0 | 0 | 0 |
| 1 | 0 | 0 | 3723 |
| 2 | 0 | 0 | 4902 |
| 3 | 0 | 0 | 5891 |
| 4 | 0 | 0 | 6450 |
| 5 | 0 | 0 | 8129 |
| 6 | 0 | 0 | 8912 |

Description of Table 9: See description of Table 6

It is derivable from Table 9 and FIG. 5 that the wild-type enzymes from Arthrobacter sp. and Bacillus megaterium do not produce (S)-tyrosine by amination p-hydroxyphenylpyruvic acid, whereas the ω-TA variant does produce (S)-tyrosine very efficiently.

5. Production of (S)-Glufosinate from 4-[hydroxy(methyl)phosphoryl]-2-Oxobutanoic Acid by ω-TA Variants Comprising Further Amino Acid Modifications

ω-TA variants having the amino acid sequence shown under SEQ ID NO 18 and ω-TA variants comprising further amino acid modifications as described herein in Table 2 were expressed together with the proteins having the activity of a DAAO and having the activity of a catalase (see “General Methods”, item 5) as described under “General Methods”, item 6, followed by spray drying as described under “General Methods”, item 9. DAAO produces 4-[hydroxy(methyl)phosphoryl]-2-oxobutanoic acid by deamination of (R)-glufosinate. 4-[hydroxy(methyl)phosphoryl]-2-oxobutanoic acid is subsequently used by ω-TA variants having further amino acid modifications as amino acceptor and converted in an amination reaction into (S)-glufosinate. Activity tests of ω-TA variants having the amino acid sequence shown under SEQ ID NO 18 and ω-TA variants comprising further amino acid modifications as described herein in Table 2 were performed according to the test described under “General Methods, item 7. The transamination reaction was monitored by HPLC-analysis as described under “General Methods” item 8 by determining the amount of (S)-glufosinate produced in each reaction 5 h after the reaction was started.

Table 10 presents the amount of the amount of (S)-glufosinate (S-GA) produced by each of the ω-TA variants comprising further amino acid modifications and the amount produced by the ω-TA variant having the amino acid sequence shown under SEQ ID NO 18.

TABLE 10

| Mutations introduced in | S-GA |
|---|---|
| respect to the amino acid | produced |
| sequence shown under | in 5 h |
| SEQ ID NO 18 | [g/l] |
| T327Q, S166G | 20.01 |
| T327Q, C384S | 19.91 |
| T327Q, E326Q | 19.66 |
| T327Q | 19.01 |
| T327Q, E326F | 18.92 |
| T327C | 18.53 |
| T327I | 17.97 |
| T327M | 17.92 |
| F164Y | 17.71 |
| F164S | 16.9 |
| T327V | 16.82 |
| T409R | 16.66 |
| T327S | 16.17 |
| V271I | 15.47 |
| S329G | 15.31 |
| T409P | 15.25 |
| L414M | 15.14 |
| Q165K | 15.09 |
| L414R | 14.42 |
| L414H | 14.32 |
| Q165C | 14.23 |
| T327V | 14.02 |
| F164C | 13.87 |
| T409K | 12.82 |
| None | 12.76 |

Description of Table 10:

For identification of amino acid changes, numbers in column 1 identify the amino acid position in the amino acid sequence shown under SEQ ID NO 18. The character appearing before the number identifies the amino acid present at the respective position in the amino acid sequence shown under SEQ ID NO 18. The character appearing after the number identifies the amino acid present at the respective position in amino acid sequences of ω-TA variants comprising further amino acid modifications. Two numbers given in the same row of column , each with a character appearing before and after the number, identifies two simultaneous amino acid substitutions (replacements) compared to the amino acid sequence shown under SEQ ID NO 18.

It is derivable from Table 10, that ω-TA variants comprising further amino acid modifications produce more (S)-glufosinate compared to ω-TA variants having the amino acid sequence shown under SEQ ID NO 18.

1. A protein having the activity of an ω-transaminase (ω-TA), wherein the protein is selected from the group consisting of:
a) proteins comprising the amino acid sequence from positions 1 to 477 as shown under SEQ ID NO 3 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
b) proteins comprising the amino acid sequence from positions 1 to 479 as shown under SEQ ID NO 6 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from T and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
c) proteins comprising the amino acid sequence from positions 1 to 476 as shown under SEQ ID NO 9 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 is different from E and the amino acid at position 187 is different from S and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
d) proteins comprising the amino acid sequence from positions 1 to 476 as shown under SEQ ID NO 12 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 and the amino acid at position 187 is different from S is different from E and the amino acid at position 197 is different from T the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P;
e) proteins comprising the amino acid sequence from positions 1 to 476 as shown under SEQ ID NO 15 apart from that the amino acid at position 25 is different from F and the amino acid at position 64 is different from L and the amino acid at position 88 is different from T and the amino acid at position 157 is different from T and the amino acid at position 165 is different from R and the amino acid at position 169 is different from V and the amino acid at position 174 and the amino acid at position 187 is different from S is different from E and the amino acid at position 197 is different from M and the amino acid at position 239 is different from S and the amino acid at position 327 is different from S and the amino acid at position 328 is different from V and the amino acid at position 384 is different from Y and the amino acid at position 389 is different from I and the amino acid at position 391 is different from D and the amino acid at position 396 is different from K and the amino acid at position 410 is different from H and the amino acid at position 414 is different from P; and
f) proteins having an amino acid sequence having at least 60%, preferably 70%, more preferably 80%, further more preferably 90%, even more preferably 95%, even further more preferably 96%, particular preferably 97%, most preferably 98% or especially preferably 99% identity with any of the amino acid sequence's shown under a), b), c), d), e) or f), given that in each case the amino acid corresponding to position 25 is different from F and the amino acid corresponding to position 64 is different from L and the amino acid corresponding to 88 is different from T and the amino acid corresponding to position 157 is different from T and the amino acid corresponding to position 165 is different from R and the amino acid corresponding to position 169 is different from V and the amino acid corresponding to position 174 is different from E and the amino acid corresponding to position 187 is different from S and the amino acid corresponding to position 197 is different from T or M and the amino acid corresponding to position 239 is different from S and the amino acid corresponding to position 327 is different from S and the amino acid corresponding to position 328 is different from V and the amino acid corresponding to position 384 is different from Y and the amino acid corresponding to position 389 is different from I and the amino acid corresponding to position 391 is different from D and the amino acid corresponding to position 396 is different from K and the amino acid corresponding to position 410 is different from H and the amino acid corresponding to position 414 is different from P.

2. The protein according to claim 1 selected from the group consisting of:
a) proteins comprising the amino acid sequence as defined in claim 1, section a) apart from that in addition the amino acid at position 2 is different from S and the amino acid at position 48 is different from D and the amino acid at position 164 is different from Y and the amino acid at position 202 is different from D and the amino acid at position 205 is different from L and the amino acid at position 242 is different from A and the amino acid at position 245 is different from A and the amino acid at position 311 is different from L and the amino acid at position 353 is different from F and the amino acid at position 359 is different from D and the amino acid at position 424 is different from K and the amino acid at position 475 is different from A and the amino acid at position 476 is different from L and the amino acid at position 477 is deleted;
b) proteins comprising the amino acid sequence as defined in claim 1, section b) apart from that in addition the amino acid at position 46 is different from T and the amino acid at position 60 is different from C and the amino acid at position 185 is different from C and the amino acid at position 186 is different from S and the amino acid at position 195 is different from S and the amino acid at position 205 is different from Y and the amino acid at position 252 is different from V and the amino acid at position 268 is different from S and the amino acid at position 409 is different from R and the amino acid at position 436 is different from A and the amino acids at positions 477 and 478 and 479 are deleted;
c) proteins comprising the amino acid sequence as defined in claim 1, section c) apart from that in addition the amino acid at position 2 is different from S and the amino acid at position 48 is different from D and the amino acid at position 69 is different from P and the amino acid at position 90 is different from S and the amino acid at position 164 is different from Y and the amino acid at position 242 is different from A and the amino acid at position 245 is different from A and the amino acid at position 268 is different from T and the amino acid at position 311 is different from L and the amino acid at position 318 is different from E and the amino acid at position 322 is different from R and the amino acid at position 353 is different from S and the amino acid at position 424 is different from K and the amino acid at position 452 is different from E;
d) proteins comprising the amino acid sequence as defined in claim 1, section d) apart from that in addition the amino acid at position 46 is different from T and the amino acid at position 60 is different from C and the amino acid at position 185 is different from C and the amino acid at position 186 is different from C and the amino acid at position 195 is different from S and the amino acid at position 205 is different from Y and the amino acid at position 252 is different from V and the amino acid at position 268 is different from S and the amino acid at position 409 is different from R and the amino acid at position 436 is different from A;
e) proteins comprising the amino acid sequence as defined in claim 1, section d) apart from that in addition the amino acid at position 48 is different from D and the amino acid at position 164 is different from Y and the amino acid at position 242 is different from A and the amino acid at position 245 is different from A and the amino acid at position 255 is different from F and the amino acid at position 424 is different from K; and
f) proteins having an amino acid sequence having at least 60% identity with any of the amino acid sequences as defined under a), b), c), d) or e) given that each amino acid position as defined under a), b), c), d) or e), respectively, is also present at the corresponding amino acid position in the amino acid sequences of the protein sequence being at least 60% identical to the amino acid sequence as defined in each of a), b), c), d) or e).

3. The protein according to claim 1, further defined as selected from the group consisting of:
a) proteins comprising the amino acid sequence from positions 1 to 476 as shown under SEQ ID NO 18;
b) proteins having an amino acid sequence having at least 60% identity with the amino acid sequence from positions 1 to 476 as shown under SEQ ID NO 18 given that the amino acids corresponding to positions 25, 64, 88, 157, 165, 169, 174, 187, 197, 239, 327, 328, 384, 389, 391, 396, 410 and 414 in SEQ ID NO 18 represent those amino acids shown at the respective positions in the amino acid sequence shown under SEQ ID NO 18; and
c) proteins having an amino acid sequence having at least 60% identity with the amino acid sequence from positions 1 to 476 as shown under SEQ ID NO 18 given that the amino acids corresponding to positions 2, 25, 46, 48, 60, 64, 69, 88, 90, 157, 164, 165, 169, 174, 187, 195, 197, 202, 205, 239, 242, 245, 252, 255, 268, 311, 318, 322, 327, 328, 353, 359, 384, 389, 391, 396, 409, 410, 414, 424, 436, 452, 475, 476 and 477 in SEQ ID NO 18 represent those amino acids shown at the respective positions in the amino acid sequence shown under SEQ ID NO 18.

4. The protein according to claim 1, further defined as selected from the group consisting of:
a) proteins according to claim 1 given that the amino acid at position 166 is G and the amino acid at position 327 is Q;
b) proteins according to claim 1 given that the amino acid at position 327 is Q and the amino acid at position 384 is S;
c) proteins according to claim 1 given that the amino acid at position 326 is Q and the amino acid at position 327 is Q;
d) proteins according to claim 1 given that the amino acid at position 327 is Q;
e) proteins according to claim 1 given that the amino acid at position 326 is F and the amino acid at position 327 is Q;
f) proteins according to claim 1 given that the amino acid at position 327 is C;
g) proteins according to claim 1 given that the amino acid at position 327 is I;
h) proteins according to claim 1 given that the amino acid at position 327 is M;
i) proteins according to claim 1 given that the amino acid at position 164 is Y;
j) proteins according to claim 1 given that the amino acid at position 164 is S;
k) proteins according to claim 1 given that the amino acid at position 327 is V;
l) proteins according to claim 1 given that the amino acid at position 409 is R;
m) proteins according to claim 1 given that the amino acid at position 327 is S;
n) proteins according to claim 1 given that the amino acid at position 271 is I;
o) proteins according to claim 1 given that the amino acid at 329 is G;
p) proteins according to claim 1 given that the amino acid at position 409 is P;
q) proteins according to claim 1 given that the amino acid at position 414 is M;
r) proteins according to claim 1 given that the amino acid at position 165 is K;
s) proteins according to claim 1 given that the amino acid at position 414 is R;
t) proteins according to claim 1 given that the amino acid at position 414 is H;
u) proteins according to claim 1 given that the amino acid at position 165 is C;
v) proteins according to claim 1 given that the amino acid at position 327 is V;
w) proteins according to claim 1 given that the amino acid at position 164 is C; and
x) proteins according to claim 1 given that the amino acid at position 409 is K.

5. The protein according to claim 4 selected from the group consisting of:
a) proteins having the amino acid sequence from positions 1 to 476 in the amino acid sequence shown under SEQ ID NO 18, apart from that the amino acid S at position 166 in SEQ ID NO 18 is substituted by G and the amino acid T at position 327 in SEQ ID NO 18 is substituted by Q;
b) proteins having the amino acid sequence from positions 1 to 476 in the amino acid sequence shown under SEQ ID NO 18, apart from that the amino acid T at position 327 in SEQ ID NO 18 is substituted by Q and the amino acid C at position 384 in SEQ ID NO 18 is substituted by S;
c) proteins having the amino acid sequence from positions 1 to 476 in the amino acid sequence shown under SEQ ID NO 18, apart from that the amino acid E at position 326 in SEQ ID NO 18 is substituted by Q and the amino acid T at position 327 in SEQ ID NO 18 is substituted by Q;
d) proteins having the amino acid sequence from positions 1 to 476 in the amino acid sequence shown under SEQ ID NO 18, apart from that the amino acid T at position 327 in SEQ ID NO 18 is substituted by Q;
e) proteins having the amino acid sequence from positions 1 to 476 in the amino acid sequence shown under SEQ ID NO 18, apart from hat the amino acid E at position 326 in SEQ ID NO 18 is substituted by F and the amino acid T at position 327 in SEQ ID NO 18 is substituted by Q;
f) proteins having the amino acid sequence from positions 1 to 476 in the amino acid sequence shown under SEQ ID NO 18, apart from that the amino acid T at position 327 in SEQ ID NO 18 is substituted by C;
g) proteins having the amino acid sequence from positions 1 to 476 in the amino acid sequence shown under SEQ ID NO 18, apart from that the amino acid T at position 327 in SEQ ID NO 18 is substituted by I;
h) proteins having the amino acid sequence from positions 1 to 476 in the amino acid sequence shown under SEQ ID NO 18, apart from that the amino acid T at position 327 in SEQ ID NO 18 is substituted by M;
i) proteins having the amino acid sequence from positions 1 to 476 in the amino acid sequence shown under SEQ ID NO 18, apart from that the amino acid F at position 164 in SEQ ID NO 18 is substituted by Y;
j) proteins having the amino acid sequence from positions 1 to 476 in the amino acid sequence shown under SEQ ID NO 18, apart from that the amino acid F at position 164 in SEQ ID NO 18 is substituted by S;
k) proteins having the amino acid sequence from positions 1 to 476 in the amino acid sequence shown under SEQ ID NO 18, apart from that the amino acid T at position 327 in SEQ ID NO 18 is substituted by V;
l) proteins having the amino acid sequence from positions 1 to 476 in the amino acid sequence shown under SEQ ID NO 18, apart from that the amino acid T at position 409 in SEQ ID NO 18 is substituted by R;
m) proteins having the amino acid sequence from positions 1 to 476 in the amino acid sequence shown under SEQ ID NO 18, apart from that the amino acid T at position 327 in SEQ ID NO 18 is substituted by S;
n) proteins having the amino acid sequence from positions 1 to 476 in the amino acid sequence shown under SEQ ID NO 18, apart from that the amino acid V at position 271 in SEQ ID NO 18 is substituted by I;
o) proteins having the amino acid sequence from positions 1 to 476 in the amino acid sequence shown under SEQ ID NO 18, apart from that the amino acid S at position 329 in SEQ ID NO 18 is substituted by G;
p) proteins having the amino acid sequence from positions 1 to 476 in the amino acid sequence shown under SEQ ID NO 18, apart from that the amino acid T at position 409 in SEQ ID NO 18 is substituted by P;
q) proteins having the amino acid sequence from positions 1 to 476 in the amino acid sequence shown under SEQ ID NO 18, apart from that the amino acid L at position 414 in SEQ ID NO 18 is substituted by M;
r) proteins having the amino acid sequence from positions 1 to 476 in the amino acid sequence shown under SEQ ID NO 18, apart from that the amino acid Q at position 165 in SEQ ID NO 18 is substituted by K;
s) proteins having the amino acid sequence from positions 1 to 476 in the amino acid sequence shown under SEQ ID NO 18, apart from that the amino acid L at position 414 in SEQ ID NO 18 is substituted by R;
t) proteins having the amino acid sequence from positions 1 to 476 in the amino acid sequence shown under SEQ ID NO 18, apart from that the amino acid L at position 414 in SEQ ID NO 18 is substituted by H;
u) proteins having the amino acid sequence from positions 1 to 476 in the amino acid sequence shown under SEQ ID NO 18, apart from that the amino acid Q at position 165 in SEQ ID NO 18 is substituted by C;
v) proteins having the amino acid sequence from positions 1 to 476 in the amino acid sequence shown under SEQ ID NO 18, apart from that the amino acid T at position 327 in SEQ ID NO 18 is substituted by V;
w) proteins having the amino acid sequence from positions 1 to 476 in the amino acid sequence shown under SEQ ID NO 18, apart from that the amino acid F at position 164 in SEQ ID NO 18 is substituted by C;
x) proteins having the amino acid sequence from positions 1 to 476 in the amino acid sequence shown under SEQ ID NO 18, apart from that the amino acid T at position 409 in SEQ ID NO 18 is substituted by K; and
y) proteins having an amino acid sequence having at least 60%, identity with any of the amino acid sequences as defined under a), b), c), d), e), f), g), h), i), j), k), l), m), n), o), p), q), r), s), t), u), v), w) or x) given that each amino acid position as defined under a), b), c), d), e), f), g), h), i), j), k), l), m), n), o), p), q), r), s), t), u), v), w) or x), respectively, is also present at the corresponding amino acid position in the amino acid sequences of the protein sequence having at least 60% identity with any of the amino acid sequences as defined under each of a), b), c), d), e), f), g), h), i), j), k), l), m), n), o), p), q), r), s), t), u), v), w) or x).

6. A nucleic acid molecule encoding a protein according to claim 1.

7. A nucleic acid molecule according to claim 6, further defined as encoding a protein having the activity of an ω-TA selected from the group consisting of:
a) nucleic acid molecules comprising the nucleic acid sequence from positions 1 to 1428 in the nucleic acid sequence as shown under SEQ ID NO 17;
b) nucleic acid molecules encoding a protein comprising the amino acid sequence from position 1 to 476 in the amino acid sequence as shown under SEQ ID NO 18;
c) nucleic acid molecules having at least 60% identity with the nucleic acid sequence from positions 1 to 1428 in the nucleic acid sequence shown under SEQ ID NO 17, given that the codon corresponding to nucleotide positions 73 to 75 in SEQ ID NO 17 has the nucleotide sequence mgn and the codon corresponding to nucleotide positions 190 to192 in SEQ ID NO 17 has the nucleotide sequence ath and the codon corresponding to nucleotide positions 262 to 264 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 493 to 495 in SEQ ID NO 17 has the nucleotide sequence mgn and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 589 to 591 in SEQ ID NO 17 has the nucleotide sequence gcn and the codon corresponding to nucleotide positions 559 to 561 in SEQ ID NO 17 has the nucleotide sequence aay and the codon corresponding to nucleotide positions 715 to 717 in SEQ ID NO 17 has the nucleotide sequence ccn and the codon corresponding to nucleotide positions 979 to 981 in SEQ ID NO 17 has the nucleotide sequence acn and the codon at nucleotide positions 982 to 984 in SEQ ID NO 17 has the nucleotide sequence ggn and the codon corresponding to nucleotide positions 1150 to 1152 in SEQ ID NO 17 has the nucleotide sequence tgy and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn;
d) nucleic acid molecules having at least 60% identity with the nucleic acid sequence from positions 1 to 1428 in the nucleic acid sequence shown under SEQ ID NO 17, given that the codon corresponding to nucleotide positions 4 to 6 in SEQ ID NO 17 has the nucleotide sequence ggn and the codon corresponding to nucleotide positions 73 to 75 in SEQ ID NO 17 has the nucleotide sequence mgn and the codon the codon corresponding to nucleotide positions 136 to 138 in SEQ ID NO 17 has the nucleotide sequence atg and the codon the codon corresponding to nucleotide positions 142 144 in SEQ ID NO 17 has the nucleotide sequence ggn and the codon corresponding to nucleotide positions 178 to 180 in SEQ ID NO 17 has the nucleotide sequence tay and the codon corresponding to nucleotide positions 190 to 192 in SEQ ID NO 17 has the nucleotide sequence ath and the codon corresponding to nucleotide positions 205 to 207 in SEQ ID NO 17 has the nucleotide sequence car and the codon corresponding to nucleotide positions 262 to 264 in SEQ ID NO 17 has the nucleotide sequence gcn and the codon corresponding to nucleotide positions 268 to 270 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 490 to 492 in SEQ ID NO 17 has the nucleotide sequence tty and the codon corresponding to nucleotide positions 493 to 495 in SEQ ID NO 17 has the nucleotide sequence car and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 553 to 555 in SEQ ID NO 17 has the nucleotide sequence tay and the codon corresponding to nucleotide positions 556 to 558 in SEQ ID NO 17 has the nucleotide sequence aay and the codon corresponding to nucleotide positions 559 to 561 in SEQ ID NO 17 has the nucleotide sequence aay and the codon corresponding to nucleotide positions 583 to 585 in SEQ ID NO 17 has the nucleotide sequence ccn and the codon corresponding to nucleotide positions 589 to 591 in SEQ ID NO 17 has the nucleotide sequence gcn and the codon corresponding to nucleotide positions 604 to 606 in SEQ ID NO 17 has the nucleotide sequence aay and the codon corresponding to nucleotide positions 613 to 615 in SEQ ID NO 17 has the nucleotide sequence tgy and the codon corresponding to nucleotide positions 715 to 717 in SEQ ID NO 17 has the nucleotide sequence ccn and the codon corresponding to nucleotide positions 724 to 726 in SEQ ID NO 17 has the nucleotide sequence gtn and the codon corresponding to nucleotide positions 733 to 735 in SEQ ID NO 17 has the nucleotide sequence acn and the codon corresponding to nucleotide positions 754 to 756 in SEQ ID NO 17 has the nucleotide sequence ath and the codon corresponding to nucleotide positions 763 to 765 in SEQ ID NO 17 has the nucleotide sequence ath and the codon corresponding to nucleotide positions 802 to 804 in SEQ ID NO 17 has the nucleotide sequence aay and the codon corresponding to nucleotide positions 931 to 933 in SEQ ID NO 17 has the nucleotide sequence gtn and the codon corresponding to nucleotide positions 952 to 954 in SEQ ID NO 17 has the nucleotide sequence gcn and the codon corresponding to nucleotide positions 964 to 966 in SEQ ID NO 17 has the nucleotide sequence aar and the codon corresponding to nucleotide positions 979 to 981 in SEQ ID NO 17 has the nucleotide sequence acn and the codon corresponding to nucleotide positions 982 to 984 in SEQ ID NO 17 has the nucleotide sequence ggn and the codon corresponding to nucleotide positions 1057 to 1059 in SEQ ID NO 17 has the nucleotide sequence ytn and the codon corresponding to nucleotide positions 1075 to 1077 in SEQ ID NO 17 has the nucleotide sequence aay and the codon corresponding to nucleotide positions 1150 to 1152 in SEQ ID NO 17 has the nucleotide sequence tay and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1225 to 1227 in SEQ ID NO 17 has the nucleotide sequence acn and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn and the codon corresponding to nucleotide positions 1270 to 1272 in SEQ ID NO 17 has the nucleotide sequence gar and the codon corresponding to nucleotide positions 1306 to 1308 in SEQ ID NO 17 has the nucleotide sequence gtn and the codon corresponding to nucleotide positions 1354 to 1356 in SEQ ID NO 17 has the nucleotide sequence ggn;
e) nucleic acid molecules hybridizing with the complementary strand of the nucleic acid molecules defined under a), b), c) or d), given that the codon corresponding to nucleotide positions 73 to 75 in SEQ ID NO 17 has the nucleotide sequence mgn and the codon corresponding to nucleotide positions 190 to192 in SEQ ID NO 17 has the nucleotide sequence ath and the codon corresponding to nucleotide positions 262 to 264 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 493 to 495 in SEQ ID NO 17 has the nucleotide sequence mgn and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 559 to 561 in SEQ ID NO 17 has the nucleotide sequence aay and the codon corresponding to nucleotide positions 715 to 717 in SEQ ID NO 17 has the nucleotide sequence ccn and the codon corresponding to nucleotide positions 979 to 981 in SEQ ID NO 17 has the nucleotide sequence acn and the codon at nucleotide positions 982 to 984 in SEQ ID NO 17 has the nucleotide sequence ggn and the codon corresponding to nucleotide positions 1150 to 1152 in SEQ ID NO 17 has the nucleotide sequence tgy and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn;
f) nucleic acid molecules hybridizing with the complemantary strand of the nucleic acid molecules defined under a), b), c) or d) given that the codon corresponding to nucleotide positions 4 to 6 in SEQ ID NO 17 has the nucleotide sequence ggn and the codon corresponding to nucleotide positions 73 to 75 in SEQ ID NO 17 has the nucleotide sequence mgn and the codon the codon corresponding to nucleotide positions 136 to 138 in SEQ ID NO 17 has the nucleotide sequence atg and the codon the codon corresponding to nucleotide positions 142 144 in SEQ ID NO 17 has the nucleotide sequence ggn and the codon corresponding to nucleotide positions 178 to 180 in SEQ ID NO 17 has the nucleotide sequence tay and the codon corresponding to nucleotide positions 190 to 192 in SEQ ID NO 17 has the nucleotide sequence ath and the codon corresponding to nucleotide positions 205 to 207 in SEQ ID NO 17 has the nucleotide sequence car and the codon corresponding to nucleotide positions 262 to 264 in SEQ ID NO 17 has the nucleotide sequence gcn and the codon corresponding to nucleotide positions 268 to 270 in SEQ ID NO 17 has the nucleotide sequence gcn and the codon corresponding to nucleotide positions 469 to 471 in SEQ ID NO 17 has the nucleotide sequence gcn and the codon corresponding to nucleotide positions 490 to 492 in SEQ ID NO 17 has the nucleotide sequence tty and the codon corresponding to nucleotide positions 493 to 495 in SEQ ID NO 17 has the nucleotide sequence car and the codon corresponding to nucleotide positions 505 to 507 in SEQ ID NO 17 has the nucleotide sequence gcn and the codon corresponding to nucleotide positions 520 to 522 in SEQ ID NO 17 has the nucleotide sequence ggn and the codon corresponding to nucleotide positions 553 to 555 in SEQ ID NO 17 has the nucleotide sequence tay and the codon corresponding to nucleotide positions 556 to 558 in SEQ ID NO 17 has the nucleotide sequence aay and the codon corresponding to nucleotide positions 559 to 561 in SEQ ID NO 17 has the nucleotide sequence aay and the codon corresponding to nucleotide positions 583 to 585 in SEQ ID NO 17 has the nucleotide sequence ccn and the codon corresponding to nucleotide positions 589 to 591 in SEQ ID NO 17 has the nucleotide sequence gcn and the codon corresponding to nucleotide positions 604 to 606 in SEQ ID NO 17 has the nucleotide sequence aay and the codon corresponding to nucleotide positions 613 to 615 in SEQ ID NO 17 has the nucleotide sequence tgy and the codon corresponding to nucleotide positions 715 to 717 in SEQ ID NO 17 has the nucleotide sequence ccn and the codon corresponding to nucleotide positions 724 to 726 in SEQ ID NO 17 has the nucleotide sequence gtn and the codon corresponding to nucleotide positions 733 to 735 in SEQ ID NO 17 has the nucleotide sequence acn and the codon corresponding to nucleotide positions 754 to 756 in SEQ ID NO 17 has the nucleotide sequence ath and the codon corresponding to nucleotide positions 763 to 765 in SEQ ID NO 17 has the nucleotide sequence ath and the codon corresponding to nucleotide positions 802 to 804 in SEQ ID NO 17 has the nucleotide sequence aay and the codon corresponding to nucleotide positions 931 to 933 in SEQ ID NO 17 has the nucleotide sequence gtn and the codon corresponding to nucleotide positions 952 to 954 in SEQ ID NO 17 has the nucleotide sequence gcn and the codon corresponding to nucleotide positions 964 to 966 in SEQ ID NO 17 has the nucleotide sequence aar and the codon corresponding to nucleotide positions 979 to 981 in SEQ ID NO 17 has the nucleotide sequence acn and the codon corresponding to nucleotide positions 982 to 984 in SEQ ID NO 17 has the nucleotide sequence ggn and the codon corresponding to nucleotide positions 1057 to 1059 in SEQ ID NO 17 has the nucleotide sequence ytn and the codon corresponding to nucleotide positions 1075 to 1077 in SEQ ID NO 17 has the nucleotide sequence aay and the codon corresponding to nucleotide positions 1150 to 1152 in SEQ ID NO 17 has the nucleotide sequence tay and the codon corresponding to nucleotide positions 1165 to 1167 in SEQ ID NO 17 has the nucleotide sequence ytn and the codon corresponding to nucleotide positions 1171 to 1173 in SEQ ID NO 17 has the nucleotide sequence gar and the codon corresponding to nucleotide positions 1186 to 1188 in SEQ ID NO 17 has the nucleotide sequence gar and the codon corresponding to nucleotide positions 1225 to 1227 in SEQ ID NO 17 has the nucleotide sequence acn and the codon corresponding to nucleotide positions 1228 to 1230 in SEQ ID NO 17 has the nucleotide sequence mgn and the codon corresponding to nucleotide positions 1240 to 1242 in SEQ ID NO 17 has the nucleotide sequence ytn and the codon corresponding to nucleotide positions 1270 to 1272 in SEQ ID NO 17 has the nucleotide sequence gar and the codon corresponding to nucleotide positions 1306 to 1308 in SEQ ID NO 17 has the nucleotide sequence gtn and the codon corresponding to nucleotide positions 1354 to 1356 in SEQ ID NO 17 has the nucleotide sequence ggn;
g) nucleic acid molecules deviating from the nucleic acid molecules defined under a), b), c), d), e) or f) due to degeneracy of the genetic code;
h) nucleic acid molecules encoding a protein having at least 60% identity with the amino acid sequence from positions 1 to 476 as shown under SEQ ID NO 18 given that the amino acids corresponding to positions 25, 64, 88, 157, 165, 169, 174, 187, 239, 327, 328, 384, 389, 391, 396, 410 and 414 in SEQ ID NO 18 represent those amino acids shown at the respective positions in the amino acid sequence shown under SEQ ID NO 18;
i) nucleic acid molecules encoding a protein having at least 60% identity with the amino acid sequence from positions 1 to 476 as shown under SEQ ID NO 18 given that the amino acids corresponding to positions 2, 25, 46, 48, 60, 64, 69, 88, 90, 157, 164, 165, 169, 174, 185, 186, 187, 195, 197, 202, 205, 239, 242, 245, 252, 255, 268, 311, 318, 322, 327, 328, 353, 359, 384, 389, 391, 396, 409, 410, 414, 424, 436, 452, 475 and 476 in SEQ ID NO 18 represent those amino acids shown at the respective positions in the amino acid sequence shown under SEQ ID NO 18; and
j) nucleic acid molecules comprising the nucleic acid sequence from positions from 1 to 1428 in the nucleic acid sequence as shown under SEQ ID NO 16.

8. A nucleic acid molecule according to claim 6 encoding a protein having the activity of an ω-TA selected from the group consisting of:
a) nucleic acid molecules comprising the nucleic acid sequence from positions 1 to 1428 in the nucleic acid sequences as shown under SEQ ID NO 16 or SEQ ID NO 17, apart from that the codon at nucleotide positions 496 to 498 in SEQ ID NO 16 or SEQ ID NO 17 has the nucleotide sequence ggn and the codon at position at nucleotide positions 979 to 981 in SEQ ID NO 16 or SEQ ID NO 17 has the nucleotide sequence car;
b) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car and the codon at nucleotide positions 1150 to 1152 in SEQ ID NO 16 or SEQ ID NO 17 has the nucleotide sequence wsn;
c) nucleic acid molecules comprising the nucleic acid sequence from positions 1 to 1428 in the nucleic acid sequences as shown under SEQ ID NO 16 or SEQ ID NO 17, apart from that the codon at nucleotide positions 976 to 978 in SEQ ID NO 16 or SEQ ID NO 17 has the nucleotide sequence car and the codon at position at nucleotide positions 979 to 981 in SEQ ID NO 16 or SEQ ID NO 17 has the nucleotide sequence car;
d) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car;
e) nucleic acid molecules comprising the nucleic acid sequence from positions 1 to 1428 in the nucleic acid sequences as shown under SEQ ID NO 16 or SEQ ID NO 17, apart from that the codon at nucleotide positions 976 to 978 in SEQ ID NO 16 or SEQ ID NO 17 has the nucleotide sequence tty and the codon at nucleotide positions 979 to 981 in SEQ ID NO 16 or SEQ ID NO 17 has the nucleotide sequence car;
f) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence car;
g) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence ath;
h) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence atg;
i) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence tay;
j) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence wsn;
k) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence gtn;
l) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence mgn;
m) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence wsn;
n) nucleic acid molecules comprising the nucleic acid sequence from positions 1 to 1428 in the nucleic acid sequences as shown under SEQ ID NO 16 or SEQ ID NO 17, apart from that the codon at nucleotide positions 811 to 813 in SEQ ID NO 16 or SEQ ID NO 17 has the nucleotide sequence ath;
o) nucleic acid molecules comprising the nucleic acid sequence from positions 1 to 1428 in the nucleic acid sequences as shown under SEQ ID NO 16 or SEQ ID NO 17, apart from that the codon at nucleotide positions 985 to 987 in SEQ ID NO 16 or SEQ ID NO 17 has the nucleotide sequence ggn;
p) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence ccn;
q) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence atg;
r) nucleic acid molecules comprising the nucleic acid sequence from positions 1 to 1428 in the nucleic acid sequences as shown under SEQ ID NO 16 or SEQ ID NO 17, apart from that the codon at nucleotide positions 493 to 495 in SEQ ID NO 16 or SEQ ID NO 17 has the nucleotide sequence aar;
s) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence mgn;
t) nucleic acid molecules comprising the nucleic acid sequence from positions 1 to 1428 in the nucleic acid sequences as shown under SEQ ID NO 16 or SEQ ID NO 17, apart from that the codon at nucleotide positions 1240 to 1242 in SEQ ID NO 16 or SEQ ID NO 17 has the nucleotide sequence cay;
u) nucleic acid molecules comprising the nucleic acid sequence from positions 1 to 1428 in the nucleic acid sequences as shown under SEQ ID NO 16 or SEQ ID NO 17, apart from that the codon at nucleotide positions 493 to 495 in SEQ ID NO 16 or SEQ ID NO 17 has the nucleotide sequence tgy;
v) nucleic acid molecules comprising the nucleic acid sequence from positions 1 to 1428 in the nucleic acid sequences as shown under SEQ ID NO 16 or SEQ ID NO 17, apart from that the codon at nucleotide positions 979 to 981 in SEQ ID NO 16 or SEQ ID NO 17 has the nucleotide sequence gtn;
w) nucleic acid molecules comprising the nucleic acid sequence from positions 1 to 1428 in the nucleic acid sequences as shown under SEQ ID NO 16 or SEQ ID NO 17, apart from that the codon at nucleotide positions 490 to 492 in SEQ ID NO 16 or SEQ ID NO 17 has the nucleotide sequence tgy;
x) nucleic acid molecules comprising the nucleic acid sequence from positions 1 to 1428 in the nucleic acid sequences as shown under SEQ ID NO 16 or SEQ ID NO 17, apart from that the codon at nucleotide positions 1225 to 1227 in SEQ ID NO 16 or SEQ ID NO 17 has the nucleotide sequence aar; and
y) nucleic acid molecules having a nucleic acid sequence having at least 60% identity with any of the nucleic acid sequences as defined under a), b), c), d), e), f), g), h), i), j), k), l), m), n), o), p), q), r), s), t), u), v), w) or x) given that each codon nucleotide sequence as defined under of a), b), c), d), e), f), g), h), i), j), k), l), m), n), o), p), q), r), s), t), u), v), w) or x), respectively, is also present at the corresponding codon nucleotide position in the nucleic acid sequences having at least 60% identity with any of the nucleic acid sequences as defined under each of a), b), c), d), e), f), g), h), i), j), k), l), m), n), o), p), q), r), s), t), u), v), w) or x).

9. A recombinant nucleic acid molecule comprising a nucleic acid molecule according to claim 6.

10. The recombinant nucleic acid molecule according to claim 9, wherein the recombinant nucleic acid molecule is a vector or a plasmid.

11. A host cell comprising a protein according to claim 1.

12. A method for the production of an amine comprising the steps of:
a) providing an amine acceptor molecule;
b) providing an amine donor molecule;
c) contacting the amine acceptor molecule provided in step a) and the amine donor molecule provided in step b) with a protein according to claim 1.

13. A method for decreasing the amount an amine enantiomer in a composition comprising (R)- and (S)-amine enantiomers, comprising the steps of
a) providing a composition comprising (R)- and (S)-amine enantiomers;
b) providing an amine acceptor molecule;
c) contacting the composition provided in step a) and the amine acceptor provided in step b) with a protein according to claim 1.

14. A host cell comprising a nucleic acid molecule according to claim 6.

15. A host cell comprising a recombinant nucleic acid molecule according to claim 9.


SEQUENCE LISTING (30 sequences)

SEQ ID NO: 1 - DNA, 1449 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (141)..(141): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (192)..(192): n is a, c, g, or t
  misc_feature (195)..(195): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (495)..(495): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (561)..(561): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (603)..(603): n is a, c, g, or t
  misc_feature (609)..(609): n is a, c, g, or t
  misc_feature (615)..(615): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (954)..(954): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1356)..(1356): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1425)..(1425): n is a, c, g, or t
  misc_feature (1428)..(1428): n is a, c, g, or t
atgwsnytnacngtncaraarathaaytgggarcargtnaargartgggaymgnaartayytnatgmgnacnttywsnacncaraaygartaycarccngtnccnathgarwsnacngarggngaytayytnathatgccngayggnacnmgnytnytngayttyttyaaycarytntaytgygtnaayytnggncaraaraaycaraargtnaaygcngcnathaargargcnytngaymgntayggnttygtntgggayacntaygcnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcnacngtnacnmgnytnmgnwsntaymgnwsnggnytngtnggngaraaywsngarwsnttywsngcncarathccnggnwsnwsntayaaywsngcngtnytnatggcnccnwsnccnaayatgttycargaywsngayggnaayytnytnaargaygaraayggngarytnytnwsngtnaartayacnmgnmgnatgathgaraaytayggnccngarcargtngcngcngtnathacngargtnwsncarggngcnggnwsngcnatgccnccntaygartayathccncarathmgnaaratgacnaargarytnggngtnytntggathaaygaygargtnytnacnggnttyggnmgnacnggnaartggttyggntaycarcaytayggngtncarccngayathathacnatgggnaarggnytnwsnwsnwsnwsnytnccngcnggngcngtnytngtnwsnaargarathgcngcnttyatggayaarcaymgntgggarwsngtnwsnacntaygcnggncayccngtngcnatggcngcngtntgygcnaayytngargtnatgatggargaraayttygtngarcargcnaargaywsnggngartayathmgnwsnaarytngarytnytncargaraarcayaarwsnathggnaayttygayggntayggnytnytntggathgtngayathgtnaaygcnaaracnaaracnccntaygtnaarytngaymgnaayttyacncayggnatgaayccnaaycarathccnacncarathathatgaaraargcnytngaraarggngtnytnathggnggngtnatgccnaayacnatgmgnathggngcnwsnytnaaygtnwsnmgnggngayathgayaargcnatggaygcnytngaytaygcnytngaytayytngarwsnggngartggcargcnytngarcaycaycaycaycaycay

SEQ ID NO: 2 - DNA, 1452 bp, Artificial Sequence
  Created by back-translation of a protein sequence.
  CDS (1)..(1452): Back translation from protein sequence shown under SEQ ID NO 3, derivable from NCBI under accesssion No. 5G09.
atgagccttaccgtacagaaaatcaactgggaacaggtcaaagaatgggatcgcaaatacctgatgcgtaccttcagcacccagaatgagtatcaacctgtcccgattgagtccacggaaggcgattacttgattatgccagatgggacgcgcttactggacttcttcaatcagctgtattgcgtgaatctggggcaaaagaaccagaaggttaatgcagcgatcaaagaagccctggatcgctatggtttcgtctgggatacgtacgcgaccgactataaagcgaaagctgcgaaaattatcattgaggacatcttgggcgatgaagattggcccggtaaagttcgctttgtgagcacaggttctgaagcagtggaaaccgccctgaatatcgcgcgcttatacactaatcgcccgctcgtagtgactcgtgagcatgactatcatggttggactggtggtgctgcgacggtcacgcgcctgcgttcgtaccgttccggacttgtgggcgaaaactcggagtccttttccgctcaaattccgggcagtagctataactccgcagttctgatggcaccgtcaccgaacatgttccaggacagtgatggcaatctcctgaaagacgaaaatggcgaactgctgtctgtgaaatatacccgtcgcatgattgaaaactatgggccggaacaggtagcagccgtcattaccgaggtttctcaaggtgccggaagcgctatgccaccgtacgaatacattccgcaaattcggaaaatgaccaaagaactgggcgtgctttggatcaatgacgaagtactgaccggttttggccgtacaggcaaatggtttggttaccagcactatggcgttcaaccggatatcattacgatgggtaaaggactgagttcatcgtcactcccagctggcgccgttctggtgagtaaagaaattgcagcgttcatggataaacatcggtgggaatctgtttcgacttatgccgggcatccagtcgcgatggcagctgtttgtgcgaacctggaagtgatgatggaagagaactttgtggaacaggcgaaagatagcggcgaatatatccgtagcaagttagagctgttgcaggagaaacacaagagcattgggaactttgacggctatgggttactgtggattgtggacattgtgaatgccaagaccaaaaccccttatgtcaaactcgaccgcaactttacacacggaatgaaccccaatcagatcccgacacagatcatcatgaagaaggcgttggagaaaggcgttctgattggtggcgtgatgcctaacacgatgcgcatcggtgcgtcgcttaacgtatcacgtggcgatattgataaagcgatggatgccctggattacgccttagattacctggagagtggtgaatggcaagccttggaacatcaccaccaccatcattaa

SEQ ID NO: 3 - protein, 483 aa, Artificial Sequence
  Synthetic Construct
MSLTVQKINWEQVKEWDRKYLMRTFSTQNEYQPVPIESTEGDYLIMPDGTRLLDFFNQLYCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSYRSGLVGENSESFSAQIPGSSYNSAVLMAPSPNMFQDSDGNLLKDENGELLSVKYTRRMIENYGPEQVAAVITEVSQGAGSAMPPYEYIPQIRKMTKELGVLWINDEVLTGFGRTGKWFGYQHYGVQPDIITMGKGLSSSSLPAGAVLVSKEIAAFMDKHRWESVSTYAGHPVAMAAVCANLEVMMEENFVEQAKDSGEYIRSKLELLQEKHKSIGNFDGYGLLWIVDIVNAKTKTPYVKLDRNFTHGMNPNQIPTQIIMKKALEKGVLIGGVMPNTMRIGASLNVSRGDIDKAMDALDYALDYLESGEWQALEHHHHHH

SEQ ID NO: 4 - DNA, 1455 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (138)..(138): n is a, c, g, or t
  misc_feature (141)..(141): n is a, c, g, or t
  misc_feature (144)..(144): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (192)..(192): n is a, c, g, or t
  misc_feature (195)..(195): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (495)..(495): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (558)..(558): n is a, c, g, or t
  misc_feature (561)..(561): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (591)..(591): n is a, c, g, or t
  misc_feature (603)..(603): n is a, c, g, or t
  misc_feature (609)..(609): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (756)..(756): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (804)..(804): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (954)..(954): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1059)..(1059): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1356)..(1356): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1428)..(1428): n is a, c, g, or t
  misc_feature (1431)..(1431): n is a, c, g, or t
  misc_feature (1434)..(1434): n is a, c, g, or t
atgggnytnacngtncaraarathaaytgggarcargtnaargartgggaymgnaartayytnatgmgnacnttywsnacncaraaygartaycarccngtnccnathgarwsnacngarggngaytayytnathacnccnggnggnacnmgnytnytngayttyttyaaycarytntgytgygtnaayytnggncaraaraaycaraargtnaaygcngcnathaargargcnytngaymgntayggnttygtntgggayacntaygcnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcnacngtnacnmgnytnmgnwsnttymgnwsnggnytngtnggngaraaywsngarwsnttywsngcncarathccnggnwsnwsntgywsnwsngcngtnytnatggcnccnwsnwsnaayacnttycargaywsnaayggnaaytayytnaargaygaraayggngarytnytnwsngtnaartayacnmgnmgnatgathgaraaytayggnccngarcargtngcngcngtnathacngargtnwsncarggngtnggnwsnacnatgccnccntaygartaygtnccncarathmgnaaratgacnaargarytnggngtnytntggathwsngaygargtnytnacnggnttyggnmgnacnggnaartggttyggntaycarcaytayggngtncarccngayathathacnatgggnaarggnytnwsnwsnwsnwsnytnccngcnggngcngtngtngtnwsnaargarathgcngcnttyatggayaarcaymgntgggarwsngtnwsnacntaygcnggncayccngtngcnatggcngcngtntgygcnaayytngargtnatgatggargaraayytngtngarcargcnaaraaywsnggngartayathmgnwsnaarytngarytnytncargaraarcayaarwsnathggnaayttygayggntayggnytnytntggathgtngayathgtnaaygcnaaracnaaracnccntaygtnaarytngaymgnaayttymgncayggnatgaayccnaaycarathccnacncarathathatggaraargcnytngaraarggngtnytnathggnggngcnatgccnaayacnatgmgnathggngcnwsnytnaaygtnwsnmgnggngayathgayaargcnatggaygcnytngaytaygcnytngaytayytngarwsnggngartggcarcarwsngcnytngarcaycaycaycaycaycay

SEQ ID NO: 5 - DNA, 1458 bp, Artificial Sequence
  Created by back-translation of a protein sequence.
  CDS (1)..(1458): Cretaed by back-translation of the protein sequence shown under SEQ ID NO 6, derivable from NCBI under accession No. 5G2P.
atgggtctgaccgtgcagaagattaactgggaacaggtgaaggagtgggatcgcaaatatctgatgcgcacgttctctacccagaacgaataccagcctgttccgattgagtcaaccgaaggtgactaccttattacacccggcggtactcgcttgctcgatttcttcaatcagctctgctgcgtgaaccttggtcaaaagaaccagaaagtaaatgccgcgatcaaagaagccctggatcgttatggctttgtgtgggacacctatgccaccgattacaaagcgaaagctgctaaaatcatcattgaggacattctgggcgatgaagattggccaggcaaagtgcgctttgtcagtaccggtagcgaagccgtggaaacggcattgaacatcgcacgcttatacaccaatcgtccacttgtggtgacacgtgagcacgattaccatggatggacgggtggagccgctactgttacacgcctgcgttcttttcggtctggcctggttggggaaaacagcgagtccttttccgctcaaattccgggcagcagctgttcgtcagcagttcttatggcgccatccagtaacacgttccaggatagcaatggcaattacctgaaggatgagaatggggaactgctgtcggtgaaatatacgcgtcgtatgatcgagaactatgggccggaacaagtggcagcggtcattaccgaagtatcccagggcgttggctcaacgatgcctccgtatgaatatgtcccgcaaatccgcaaaatgaccaaagaactgggagtactctggatttccgacgaagtactgactggctttggtcgcaccggcaaatggttcggttatcagcactacggagtgcaacccgacatcattacaatgggtaaaggtctgagtagcagcagcttaccggcaggggccgtagtcgtttcgaaagaaattgctgcgtttatggataaacaccgctgggaatcggttagcacttatgccggtcatccggttgcgatggcagcggtctgtgctaacctcgaagtgatgatggaagagaatctggttgagcaagccaagaactctggtgaatacatccgctcaaaactggaattactgcaggaaaagcacaaatcgattgggaactttgatggctatgggctgttatggatcgtagacattgtcaacgcgaaaaccaaaaccccgtatgtgaaattggaccgcaatttccgtcatggcatgaatccgaaccagattcctactcagatcattatggagaaagcgttggagaagggcgtcttgattggcggtgcaatgccgaatacgatgcggatcggtgccagtctgaatgtctctcgtggcgatattgataaagcgatggatgcgctggactatgcgctggactacctggaaagcggcgaatggcaacagagtgcgttagaacatcaccatcatcaccattaa

SEQ ID NO: 6 - protein, 485 aa, Artificial Sequence
  Synthetic Construct
MGLTVQKINWEQVKEWDRKYLMRTFSTQNEYQPVPIESTEGDYLITPGGTRLLDFFNQLCCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSFRSGLVGENSESFSAQIPGSSCSSAVLMAPSSNTFQDSNGNYLKDENGELLSVKYTRRMIENYGPEQVAAVITEVSQGVGSTMPPYEYVPQIRKMTKELGVLWISDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLELLQEKHKSIGNFDGYGLLWIVDIVNAKTKTPYVKLDRNFRHGMNPNQIPTQIIMEKALEKGVLIGGAMPNTMRIGASLNVSRGDIDKAMDALDYALDYLESGEWQQSALEHHHHHH

SEQ ID NO: 7 - DNA, 1428 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (141)..(141): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (192)..(192): n is a, c, g, or t
  misc_feature (195)..(195): n is a, c, g, or t
  misc_feature (207)..(207): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (495)..(495): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (561)..(561): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (603)..(603): n is a, c, g, or t
  misc_feature (609)..(609): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (804)..(804): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (966)..(966): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1428)..(1428): n is a, c, g, or t
atgwsnytnacngtncaraarathaaytgggarcargtnaargartgggaymgnaartayytnatgmgnacnttywsnacncaraaygartaycarccngtnccnathgarwsnacngarggngaytayytnathatgccngayggnacnmgnytnytngayttyttyaaycarytntaytgygtnaayytnggncaraaraayccnaargtnaaygcngcnathaargargcnytngaymgntayggnttygtntgggayacntaywsnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcnacngtnacnmgnytnmgnwsntaymgnwsnggnytngtnggngaraaywsngarwsnttywsngcncarathccnggnwsnwsntayaaywsngcngtnytnatggcnccnwsnccnaayatgttycargaywsnaayggnaaytgyytnaargaygaraayggngarytnytnwsngtnaartayacnmgnmgnatgathgaraaytayggnccngarcargtngcngcngtnathacngargtnwsncarggngcnggnwsngcnatgccnccntaygartayathccncarathmgnaaratgacnaargarytnggngtnytntggathacngaygargtnytnacnggnttyggnmgnacnggnaartggttyggntaycarcaytayggngtncarccngayathathacnatgggnaarggnytnwsnwsnwsnwsnytnccngcnggngcngtnytngtnwsnaargarathgcngarttyatggaymgncaymgntgggarwsngtnwsnacntaygcnggncayccngtngcnatggcngcngtntgygcnaayytngargtnatgatggargaraayttygtngarcargcnaaraaywsnggngartayathmgnwsnaarytngarytnytncargaraarcayaarwsnathggnaayttygayggntayggnytnytntggathgtngayathgtnaaygcnaaracnaaracnccntaygtnaarytngaymgnaayttyacncayggnatgaayccnaaycarathccnacncarathathatgaaraargcnytngaraarggngtnytnathggnggngtnatgccnaayacnatgmgnathggngcnwsnytnaaygtnwsnmgngargayathgayaargcnatggaygcnytngaytaygcnytngaytayytngarwsnggngartggcarcarwsn

SEQ ID NO: 8 - DNA, 1431 bp, Bacillus spec. Soil768D1
  CDS (1)..(1428)
  ref <308>: DATABASE ACCESSION NUMBER: LMTA01000079.1
  ref <309>: DATABASE ENTRY DATE: 2015-11-02
  ref <313>: RELEVANT RESIDUES IN SEQ ID NO: (118099)..(119529)
atgagtttaacagtgcaaaaaataaactgggagcaagttaaagaatgggatcgtaaatacctgatgaggacgtttagtacccaaaatgagtatcaaccagtaccgattgaatcaacagaaggtgactacttgattatgcctgatggcacaagattattagatttcttcaatcaactttattgtgtcaatttaggtcagaaaaatccaaaagttaatgctgcaatcaaggaggctttagaccgatatggttttgtatgggatacttattcaactgattataaagccaaagcagcaaagataattattgaggatattttaggtgatgaagactggccaggaaaagttaggttcgtatcaacaggaagtgaagcagtggaaacggcactgaacatcgcgaggttatatacaaatcgcccactagtggttacacgagaacacgattatcatggttggactggtggagctgctactgttactcgattaaggtcatatcgaagtggtttagttggggaaaattcagaatctttttcagcacaaatacccggttcatcatataatagtgctgttttgatggcgccatcccctaacatgtttcaggattcgaacggcaactgcctaaaagatgaaaacggggaattgttgagtgtaaagtatacacgtcgtatgatcgaaaactatggtccggaacaagtggcagcagtgataactgaagtatcgcaaggtgcaggctctgctatgcccccatatgaatacattccacagatccgaaaaatgacaaaagaactaggtgtcctttggattactgatgaagttcttactggctttgggcggacagggaagtggtttggatatcagcattatggggtacagccagatataatcactatgggtaaaggactctccagttcctcactccctgctggcgctgtcttagttagtaaggaaattgcagagtttatggataggcaccgatgggagtcagtatccacctatgccggtcatccagttgcgatggctgcggtctgtgcaaatttagaagtgatgatggaagaaaactttgttgagcaagcgaagaacagcggcgagtatataaggagtaaacttgaacttctgcaagaaaaacataaaagtataggtaattttgacggatatggccttttatggatagtagatattgttaatgccaagactaagactccttacgtgaaattggaccggaactttacgcacgggatgaatccaaatcaaatcccaacacaaatcattatgaaaaaagcgctagaaaaaggagtgctaattggtggagtaatgcctaatacaatgagaattggcgcatctttgaatgttagtcgcgaagacatcgataaagcaatggatgcactggattatgcacttgactatttggaaagtggagaatggcagcaatcctaa

SEQ ID NO: 9 - protein, 476 aa, Bacillus spec. Soil768D1
MSLTVQKINWEQVKEWDRKYLMRTFSTQNEYQPVPIESTEGDYLIMPDGTRLLDFFNQLYCVNLGQKNPKVNAAIKEALDRYGFVWDTYSTDYKAKAAKIIIEDILGDEDWPGKVRFVSTGSEAVETALNIARLYTNRPLVVTREHDYHGWTGGAATVTRLRSYRSGLVGENSESFSAQIPGSSYNSAVLMAPSPNMFQDSNGNCLKDENGELLSVKYTRRMIENYGPEQVAAVITEVSQGAGSAMPPYEYIPQIRKMTKELGVLWITDEVLTGFGRTGKWFGYQHYGVQPDIITMGKGLSSSSLPAGAVLVSKEIAEFMDRHRWESVSTYAGHPVAMAAVCANLEVMMEENFVEQAKNSGEYIRSKLELLQEKHKSIGNFDGYGLLWIVDIVNAKTKTPYVKLDRNFTHGMNPNQIPTQIIMKKALEKGVLIGGVMPNTMRIGASLNVSREDIDKAMDALDYALDYLESGEWQQS

SEQ ID NO: 10 - DNA, 1428 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (138)..(138): n is a, c, g, or t
  misc_feature (141)..(141): n is a, c, g, or t
  misc_feature (144)..(144): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (192)..(192): n is a, c, g, or t
  misc_feature (195)..(195): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (495)..(495): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (558)..(558): n is a, c, g, or t
  misc_feature (561)..(561): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (591)..(591): n is a, c, g, or t
  misc_feature (603)..(603): n is a, c, g, or t
  misc_feature (609)..(609): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (756)..(756): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (804)..(804): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (954)..(954): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1059)..(1059): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1356)..(1356): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1428)..(1428): n is a, c, g, or t
atgggnytnacngtncaraarathaaytgggarcargtnaargartgggaymgnaartayytnatgmgnacnttywsnacncaraaygartaycarccngtnccnathgarwsnacngarggngaytayytnathacnccnggnggnacnmgnytnytngayttyttyaaycarytntgytgygtnaayytnggncaraaraaycaraargtnaaygcngcnathaargargcnytngaymgntayggnttygtntgggayacntaygcnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcnacngtnacnmgnytnmgnwsnttymgnwsnggnytngtnggngaraaywsngarwsnttywsngcncarathccnggnwsnwsntgywsnwsngcngtnytnatggcnccnwsnwsnaayacnttycargaywsnaayggnaaytayytnaargaygaraayggngarytnytnwsngtnaartayacnmgnmgnatgathgaraaytayggnccngarcargtngcngcngtnathacngargtnwsncarggngtnggnwsnacnatgccnccntaygartaygtnccncarathmgnaaratgacnaargarytnggngtnytntggathwsngaygargtnytnacnggnttyggnmgnacnggnaartggttyggntaycarcaytayggngtncarccngayathathacnatgggnaarggnytnwsnwsnwsnwsnytnccngcnggngcngtngtngtnwsnaargarathgcngcnttyatggayaarcaymgntgggarwsngtnwsnacntaygcnggncayccngtngcnatggcngcngtntgygcnaayytngargtnatgatggargaraayytngtngarcargcnaaraaywsnggngartayathmgnwsnaarytngarytnytncargaraarcayaarwsnathggnaayttygayggntayggnytnytntggathgtngayathgtnaaygcnaaracnaaracnccntaygtnaarytngaymgnaayttymgncayggnatgaayccnaaycarathccnacncarathathatggaraargcnytngaraarggngtnytnathggnggngcnatgccnaayacnatgmgnathggngcnwsnytnaaygtnwsnmgnggngayathgayaargcnatggaygcnytngaytaygcnytngaytayytngarwsnggngartggcarcarwsn

SEQ ID NO: 11 - DNA, 1431 bp, Artificial Sequence
  Mutated wild-type sequence.
  CDS (1)..(1431)
  ref <308>: DATABASE ACCESSION NUMBER: NRP_ABN37900
  ref <309>: DATABASE ENTRY DATE: 2006-10-12
  ref <313>: RELEVANT RESIDUES IN SEQ ID NO: (1)..(1431)
  ref <302>: TITLE: Thermostable omega-tranaminase
  ref <308>: DATABASE ACCESSION NUMBER: NRP_ABN37900
  ref <309>: DATABASE ENTRY DATE: 2006-10-12
  ref <310>: PATENT DOCUMENT NUMBER: WO 2006/063336 A2
  ref <311>: PATENT FILING DATE: 2005-12-12
  ref <313>: RELEVANT RESIDUES IN SEQ ID NO: (1)..(1431)
atgggtctgaccgtgcagaaaattaattgggaacaggtgaaagaatgggatcgcaaatatctgatgcgtacctttagcacccagaatgaatatcagccggttccgattgaaagcaccgaaggcgattatctgattacaccgggtggtacacgtctgctggatttttttaatcagctgtgctgtgttaatctgggtcagaaaaatcagaaagtgaatgcagccattaaagaagcactggatcgttacggttttgtgtgggatacctatgccaccgattataaagcaaaagccgcaaaaattattattgaagatattctgggagatgaagattggcctggtaaagttcgttttgttagcaccggtagcgaagcagttgaaaccgcactgaatattgcacgtctgtataccaatcgtccgctggttgttacccgtgaacatgattatcatggttggaccggtggtgcagcaaccgttacccgtctgcgtagctttcgtagcggtctggttggtgaaaatagcgaaagctttagcgcacagattccgggtagcagctgtagcagcgcagttctgatggcaccgagcagcaatacctttcaggatagcaatggcaattatctgaaagatgaaaacggtgaactgctgtctgttaaatatacccgtcgcatgattgaaaattatggtccggaacaggttgcagcagttattaccgaagttagccagggtgttggtagcaccatgcctccgtatgaatatgttccgcagattcgcaaaatgaccaaagaactgggtgttctgtggatttctgatgaagttctgaccggttttggtcgtaccggtaaatggtttggctatcagcattatggtgttcagccggatattattaccatgggtaaaggtctgagcagcagcagcctgcctgcaggtgcagttgttgtgagcaaagaaatcgcagcctttatggataaacatcgttgggaaagcgttagcacctatgcaggtcatcctgtggctatggctgccgtttgtgcaaatctggaagtgatgatggaagaaaatctggttgaacaggccaaaaatagcggtgaatatatccgtagcaaactggaactgctgcaggaaaaacataaaagcattggcaattttgatggttatggcctgctgtggattgttgatattgtgaatgccaaaaccaaaaccccgtatgttaaactggatcgcaattttcgtcatggcatgaatccgaatcagattccgacccagatcattatggaaaaagccctggaaaaaggtgttctgattggtggtgcaatgccgaataccatgcgtattggtgcaagcctgaatgttagccgtggcgatattgataaagcaatggatgcactggattatgccctggattatctggaaagcggtgaatggcagcagagctaa

SEQ ID NO: 12 - protein, 476 aa, Artificial Sequence
  Synthetic Construct
MGLTVQKINWEQVKEWDRKYLMRTFSTQNEYQPVPIESTEGDYLITPGGTRLLDFFNQLCCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSFRSGLVGENSESFSAQIPGSSCSSAVLMAPSSNTFQDSNGNYLKDENGELLSVKYTRRMIENYGPEQVAAVITEVSQGVGSTMPPYEYVPQIRKMTKELGVLWISDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLELLQEKHKSIGNFDGYGLLWIVDIVNAKTKTPYVKLDRNFRHGMNPNQIPTQIIMEKALEKGVLIGGAMPNTMRIGASLNVSRGDIDKAMDALDYALDYLESGEWQQS

SEQ ID NO: 13 - DNA, 1428 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (141)..(141): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (192)..(192): n is a, c, g, or t
  misc_feature (195)..(195): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (495)..(495): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (561)..(561): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (603)..(603): n is a, c, g, or t
  misc_feature (609)..(609): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (954)..(954): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1059)..(1059): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1356)..(1356): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1428)..(1428): n is a, c, g, or t
atgggnytnacngtncaraarathaaytgggarcargtnaargartgggaymgnaartayytnatgmgnacnttywsnacncaraaygartaycarccngtnccnathgarwsnacngarggngaytayytnathatgccngayggnacnmgnytnytngayttyttyaaycarytntaytgygtnaayytnggncaraaraaycaraargtnaaygcngcnathaargargcnytngaymgntayggnttygtntgggayacntaygcnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcnacngtnacnmgnytnmgnwsntaymgnwsnggnytngtnggngaraaywsngarwsnttywsngcncarathccnggnwsnwsntayaaywsngcngtnytnatggcnccnwsnccnaayatgttycargaywsnaayggnaaytgyytnaargaygaraayggngarytnytnwsngtnaartayacnmgnmgnatgathgaraaytayggnccngarcargtngcngcngtnathacngargtnwsncarggngcnggnwsngcnatgccnccntaygartayathccncarttymgnaaratgacnaargarytnggngtnytntggathaaygaygargtnytnacnggnttyggnmgnacnggnaartggttyggntaycarcaytayggngtncarccngayathathacnatgggnaarggnytnwsnwsnwsnwsnytnccngcnggngcngtngtngtnwsnaargarathgcngcnttyatggayaarcaymgntgggarwsngtnwsnacntaygcnggncayccngtngcnatggcngcngtntgygcnaayytngargtnatgatggargaraayytngtngarcargcnaaraaywsnggngartayathmgnwsnaarytngarytnytncargaraarcayaarwsnathggnaayttygayggntayggnytnytntggathgtngayathgtnaaygcnaaracnaaracnccntaygtnaarytngaymgnaayttyacncayggnatgaayccnaaycarathccnacncarathathatgaaraargcnytngaraarggngtnytnathggnggngtnatgccnaayacnatgmgnathggngcnwsnytnaaygtnwsnmgnggngayathgayaargcnatggaygcnytngaytaygcnytngaytayytngarwsnggngartggcarcarwsn

SEQ ID NO: 14 - DNA, 1431 bp, Arthrobacter spec.
  CDS (1)..(1431)
  ref <302>: TITLE: Thermostable omega-transaminase
  ref <308>: DATABASE ACCESSION NUMBER: NRN_EA065097
  ref <309>: DATABASE ENTRY DATE: 2006-10-12
  ref <310>: PATENT DOCUMENT NUMBER: WO 2006/063336 A2
  ref <311>: PATENT FILING DATE: 2005-12-12
  ref <313>: RELEVANT RESIDUES IN SEQ ID NO: (1)..(1431)
atgggtttaacagtgcaaaaaataaactgggagcaagttaaagaatgggatcgtaaatacctgatgaggacgtttagtacccaaaatgagtatcaacctgtaccgattgaatcaacagaaggtgactatttgattatgcctgatggcacaagattattagatttcttcaatcaactttattgtgtcaatttaggccagaaaaatcaaaaagttaatgctgcaatcaaggaggctttagaccgatatggttttgtatgggatacttatgcaactgattataaagccaaagcagccaagataattattgaggatattttaggtgatgaagactggccaggaaaagttaggttcgtatcaacaggaagtgaagcagtggaaacggcactgaatatcgcgaggttatatacaaatcgcccactagtggttacacgagaacacgattatcatggttggactggtggagctgctactgttactcgattaaggtcatatcgaagtggtttagttggggaaaattcagaatctttttcagcacaaatacccggttcatcatataatagtgctgttttgatggcgccatcccctaacatgtttcaggattcgaacggcaactgcctaaaagatgaaaacggggaattgttgagtgtaaagtatacacgtcgtatgatcgaaaactatggtccggaacaagtggcagcagtgataactgaagtatcgcaaggtgcaggctctgctatgcccccatatgaatacattccacaattccgaaaaatgacaaaagaactaggtgtcctttggattaatgatgaagttcttactggctttgggcgtacagggaagtggtttggatatcagcattatggggtacagccagatataatcactatgggtaaaggactctccagttcctcactccctgctggcgctgtcgtagttagtaaggaaattgcagcgtttatggataagcaccgatgggagtcagtatccacctatgctggtcatccagttgcgatggctgcggtttgtgcaaatttagaagtaatgatggaagaaaaccttgttgagcaagcgaagaacagcggcgagtatataaggagtaaacttgaacttctgcaagaaaagcataaaagtataggcaatttcgacggatatggccttttatggatagtagatattgtgaatgccaagactaagactccttacgtaaaattggacaggaactttacgcacgggatgaatccaaatcaaatcccaacacaaatcattatgaaaaaagcgctagaaaaaggagtgctgattggtggagtaatgcctaatacaatgagaattggcgcatctttgaatgttagtcgcggagacatcgataaagcaatggatgcactggattatgcacttgactatttagaaagtggagaatggcagcaatcctaa

SEQ ID NO: 15 - protein, 476 aa, Arthrobacter spec.
MGLTVQKINWEQVKEWDRKYLMRTFSTQNEYQPVPIESTEGDYLIMPDGTRLLDFFNQLYCVNLGQKNQKVNAAIKEALDRYGFVWDTYATDYKAKAAKIIIEDILGDEDWPGKVRFVSTGSEAVETALNIARLYTNRPLVVTREHDYHGWTGGAATVTRLRSYRSGLVGENSESFSAQIPGSSYNSAVLMAPSPNMFQDSNGNCLKDENGELLSVKYTRRMIENYGPEQVAAVITEVSQGAGSAMPPYEYIPQFRKMTKELGVLWINDEVLTGFGRTGKWFGYQHYGVQPDIITMGKGLSSSSLPAGAVVVSKEIAAFMDKHRWESVSTYAGHPVAMAAVCANLEVMMEENLVEQAKNSGEYIRSKLELLQEKHKSIGNFDGYGLLWIVDIVNAKTKTPYVKLDRNFTHGMNPNQIPTQIIMKKALEKGVLIGGVMPNTMRIGASLNVSRGDIDKAMDALDYALDYLESGEWQQS

SEQ ID NO: 16 - DNA, 1446 bp, Artificial Sequence
  Created by back-translation of a protein sequence due to degeneracy of genetic code.
  misc_feature (6)..(6): n is a, c, g, or t
  misc_feature (9)..(9): n is a, c, g, or t
  misc_feature (12)..(12): n is a, c, g, or t
  misc_feature (15)..(15): n is a, c, g, or t
  misc_feature (39)..(39): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (72)..(72): n is a, c, g, or t
  misc_feature (75)..(75): n is a, c, g, or t
  misc_feature (78)..(78): n is a, c, g, or t
  misc_feature (81)..(81): n is a, c, g, or t
  misc_feature (99)..(99): n is a, c, g, or t
  misc_feature (102)..(102): n is a, c, g, or t
  misc_feature (105)..(105): n is a, c, g, or t
  misc_feature (114)..(114): n is a, c, g, or t
  misc_feature (117)..(117): n is a, c, g, or t
  misc_feature (123)..(123): n is a, c, g, or t
  misc_feature (132)..(132): n is a, c, g, or t
  misc_feature (141)..(141): n is a, c, g, or t
  misc_feature (144)..(144): n is a, c, g, or t
  misc_feature (147)..(147): n is a, c, g, or t
  misc_feature (150)..(150): n is a, c, g, or t
  misc_feature (153)..(153): n is a, c, g, or t
  misc_feature (156)..(156): n is a, c, g, or t
  misc_feature (159)..(159): n is a, c, g, or t
  misc_feature (177)..(177): n is a, c, g, or t
  misc_feature (186)..(186): n is a, c, g, or t
  misc_feature (195)..(195): n is a, c, g, or t
  misc_feature (213)..(213): n is a, c, g, or t
  misc_feature (219)..(219): n is a, c, g, or t
  misc_feature (222)..(222): n is a, c, g, or t
  misc_feature (234)..(234): n is a, c, g, or t
  misc_feature (237)..(237): n is a, c, g, or t
  misc_feature (243)..(243): n is a, c, g, or t
  misc_feature (249)..(249): n is a, c, g, or t
  misc_feature (255)..(255): n is a, c, g, or t
  misc_feature (264)..(264): n is a, c, g, or t
  misc_feature (270)..(270): n is a, c, g, or t
  misc_feature (273)..(273): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (318)..(318): n is a, c, g, or t
  misc_feature (321)..(321): n is a, c, g, or t
  misc_feature (336)..(336): n is a, c, g, or t
  misc_feature (339)..(339): n is a, c, g, or t
  misc_feature (345)..(345): n is a, c, g, or t
  misc_feature (348)..(348): n is a, c, g, or t
  misc_feature (354)..(354): n is a, c, g, or t
  misc_feature (357)..(357): n is a, c, g, or t
  misc_feature (360)..(360): n is a, c, g, or t
  misc_feature (363)..(363): n is a, c, g, or t
  misc_feature (366)..(366): n is a, c, g, or t
  misc_feature (372)..(372): n is a, c, g, or t
  misc_feature (375)..(375): n is a, c, g, or t
  misc_feature (381)..(381): n is a, c, g, or t
  misc_feature (384)..(384): n is a, c, g, or t
  misc_feature (387)..(387): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (408)..(408): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (423)..(423): n is a, c, g, or t
  misc_feature (426)..(426): n is a, c, g, or t
  misc_feature (429)..(429): n is a, c, g, or t
  misc_feature (432)..(432): n is a, c, g, or t
  misc_feature (450)..(450): n is a, c, g, or t
  misc_feature (456)..(456): n is a, c, g, or t
  misc_feature (459)..(459): n is a, c, g, or t
  misc_feature (462)..(462): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (480)..(480): n is a, c, g, or t
  misc_feature (483)..(483): n is a, c, g, or t
  misc_feature (486)..(486): n is a, c, g, or t
  misc_feature (489)..(489): n is a, c, g, or t
  misc_feature (498)..(498): n is a, c, g, or t
  misc_feature (501)..(501): n is a, c, g, or t
  misc_feature (504)..(504): n is a, c, g, or t
  misc_feature (507)..(507): n is a, c, g, or t
  misc_feature (510)..(510): n is a, c, g, or t
  misc_feature (519)..(519): n is a, c, g, or t
  misc_feature (522)..(522): n is a, c, g, or t
  misc_feature (525)..(525): n is a, c, g, or t
  misc_feature (531)..(531): n is a, c, g, or t
  misc_feature (534)..(534): n is a, c, g, or t
  misc_feature (543)..(543): n is a, c, g, or t
  misc_feature (546)..(546): n is a, c, g, or t
  misc_feature (549)..(549): n is a, c, g, or t
  misc_feature (552)..(552): n is a, c, g, or t
  misc_feature (564)..(564): n is a, c, g, or t
  misc_feature (567)..(567): n is a, c, g, or t
  misc_feature (570)..(570): n is a, c, g, or t
  misc_feature (576)..(576): n is a, c, g, or t
  misc_feature (579)..(579): n is a, c, g, or t
  misc_feature (582)..(582): n is a, c, g, or t
  misc_feature (585)..(585): n is a, c, g, or t
  misc_feature (591)..(591): n is a, c, g, or t
  misc_feature (603)..(603): n is a, c, g, or t
  misc_feature (609)..(609): n is a, c, g, or t
  misc_feature (618)..(618): n is a, c, g, or t
  misc_feature (633)..(633): n is a, c, g, or t
  misc_feature (639)..(639): n is a, c, g, or t
  misc_feature (642)..(642): n is a, c, g, or t
  misc_feature (645)..(645): n is a, c, g, or t
  misc_feature (648)..(648): n is a, c, g, or t
  misc_feature (657)..(657): n is a, c, g, or t
  misc_feature (660)..(660): n is a, c, g, or t
  misc_feature (663)..(663): n is a, c, g, or t
  misc_feature (681)..(681): n is a, c, g, or t
  misc_feature (684)..(684): n is a, c, g, or t
  misc_feature (693)..(693): n is a, c, g, or t
  misc_feature (696)..(696): n is a, c, g, or t
  misc_feature (699)..(699): n is a, c, g, or t
  misc_feature (702)..(702): n is a, c, g, or t
  misc_feature (708)..(708): n is a, c, g, or t
  misc_feature (714)..(714): n is a, c, g, or t
  misc_feature (717)..(717): n is a, c, g, or t
  misc_feature (723)..(723): n is a, c, g, or t
  misc_feature (726)..(726): n is a, c, g, or t
  misc_feature (729)..(729): n is a, c, g, or t
  misc_feature (732)..(732): n is a, c, g, or t
  misc_feature (735)..(735): n is a, c, g, or t
  misc_feature (741)..(741): n is a, c, g, or t
  misc_feature (744)..(744): n is a, c, g, or t
  misc_feature (759)..(759): n is a, c, g, or t
  misc_feature (768)..(768): n is a, c, g, or t
  misc_feature (777)..(777): n is a, c, g, or t
  misc_feature (786)..(786): n is a, c, g, or t
  misc_feature (789)..(789): n is a, c, g, or t
  misc_feature (792)..(792): n is a, c, g, or t
  misc_feature (795)..(795): n is a, c, g, or t
  misc_feature (813)..(813): n is a, c, g, or t
  misc_feature (816)..(816): n is a, c, g, or t
  misc_feature (819)..(819): n is a, c, g, or t
  misc_feature (822)..(822): n is a, c, g, or t
  misc_feature (828)..(828): n is a, c, g, or t
  misc_feature (831)..(831): n is a, c, g, or t
  misc_feature (834)..(834): n is a, c, g, or t
  misc_feature (837)..(837): n is a, c, g, or t
  misc_feature (849)..(849): n is a, c, g, or t
  misc_feature (864)..(864): n is a, c, g, or t
  misc_feature (867)..(867): n is a, c, g, or t
  misc_feature (873)..(873): n is a, c, g, or t
  misc_feature (885)..(885): n is a, c, g, or t
  misc_feature (891)..(891): n is a, c, g, or t
  misc_feature (897)..(897): n is a, c, g, or t
  misc_feature (900)..(900): n is a, c, g, or t
  misc_feature (903)..(903): n is a, c, g, or t
  misc_feature (906)..(906): n is a, c, g, or t
  misc_feature (909)..(909): n is a, c, g, or t
  misc_feature (912)..(912): n is a, c, g, or t
  misc_feature (915)..(915): n is a, c, g, or t
  misc_feature (918)..(918): n is a, c, g, or t
  misc_feature (921)..(921): n is a, c, g, or t
  misc_feature (924)..(924): n is a, c, g, or t
  misc_feature (927)..(927): n is a, c, g, or t
  misc_feature (930)..(930): n is a, c, g, or t
  misc_feature (933)..(933): n is a, c, g, or t
  misc_feature (936)..(936): n is a, c, g, or t
  misc_feature (939)..(939): n is a, c, g, or t
  misc_feature (951)..(951): n is a, c, g, or t
  misc_feature (954)..(954): n is a, c, g, or t
  misc_feature (972)..(972): n is a, c, g, or t
  misc_feature (981)..(981): n is a, c, g, or t
  misc_feature (984)..(984): n is a, c, g, or t
  misc_feature (987)..(987): n is a, c, g, or t
  misc_feature (990)..(990): n is a, c, g, or t
  misc_feature (996)..(996): n is a, c, g, or t
  misc_feature (999)..(999): n is a, c, g, or t
  misc_feature (1005)..(1005): n is a, c, g, or t
  misc_feature (1008)..(1008): n is a, c, g, or t
  misc_feature (1011)..(1011): n is a, c, g, or t
  misc_feature (1017)..(1017): n is a, c, g, or t
  misc_feature (1020)..(1020): n is a, c, g, or t
  misc_feature (1023)..(1023): n is a, c, g, or t
  misc_feature (1029)..(1029): n is a, c, g, or t
  misc_feature (1035)..(1035): n is a, c, g, or t
  misc_feature (1041)..(1041): n is a, c, g, or t
  misc_feature (1059)..(1059): n is a, c, g, or t
  misc_feature (1062)..(1062): n is a, c, g, or t
  misc_feature (1071)..(1071): n is a, c, g, or t
  misc_feature (1080)..(1080): n is a, c, g, or t
  misc_feature (1083)..(1083): n is a, c, g, or t
  misc_feature (1095)..(1095): n is a, c, g, or t
  misc_feature (1098)..(1098): n is a, c, g, or t
  misc_feature (1104)..(1104): n is a, c, g, or t
  misc_feature (1110)..(1110): n is a, c, g, or t
  misc_feature (1113)..(1113): n is a, c, g, or t
  misc_feature (1131)..(1131): n is a, c, g, or t
  misc_feature (1137)..(1137): n is a, c, g, or t
  misc_feature (1149)..(1149): n is a, c, g, or t
  misc_feature (1155)..(1155): n is a, c, g, or t
  misc_feature (1158)..(1158): n is a, c, g, or t
  misc_feature (1161)..(1161): n is a, c, g, or t
  misc_feature (1167)..(1167): n is a, c, g, or t
  misc_feature (1170)..(1170): n is a, c, g, or t
  misc_feature (1179)..(1179): n is a, c, g, or t
  misc_feature (1185)..(1185): n is a, c, g, or t
  misc_feature (1191)..(1191): n is a, c, g, or t
  misc_feature (1197)..(1197): n is a, c, g, or t
  misc_feature (1200)..(1200): n is a, c, g, or t
  misc_feature (1206)..(1206): n is a, c, g, or t
  misc_feature (1212)..(1212): n is a, c, g, or t
  misc_feature (1218)..(1218): n is a, c, g, or t
  misc_feature (1227)..(1227): n is a, c, g, or t
  misc_feature (1230)..(1230): n is a, c, g, or t
  misc_feature (1233)..(1233): n is a, c, g, or t
  misc_feature (1242)..(1242): n is a, c, g, or t
  misc_feature (1254)..(1254): n is a, c, g, or t
  misc_feature (1257)..(1257): n is a, c, g, or t
  misc_feature (1278)..(1278): n is a, c, g, or t
  misc_feature (1281)..(1281): n is a, c, g, or t
  misc_feature (1290)..(1290): n is a, c, g, or t
  misc_feature (1293)..(1293): n is a, c, g, or t
  misc_feature (1296)..(1296): n is a, c, g, or t
  misc_feature (1302)..(1302): n is a, c, g, or t
  misc_feature (1305)..(1305): n is a, c, g, or t
  misc_feature (1308)..(1308): n is a, c, g, or t
  misc_feature (1314)..(1314): n is a, c, g, or t
  misc_feature (1320)..(1320): n is a, c, g, or t
  misc_feature (1326)..(1326): n is a, c, g, or t
  misc_feature (1332)..(1332): n is a, c, g, or t
  misc_feature (1335)..(1335): n is a, c, g, or t
  misc_feature (1338)..(1338): n is a, c, g, or t
  misc_feature (1341)..(1341): n is a, c, g, or t
  misc_feature (1347)..(1347): n is a, c, g, or t
  misc_feature (1350)..(1350): n is a, c, g, or t
  misc_feature (1353)..(1353): n is a, c, g, or t
  misc_feature (1356)..(1356): n is a, c, g, or t
  misc_feature (1371)..(1371): n is a, c, g, or t
  misc_feature (1380)..(1380): n is a, c, g, or t
  misc_feature (1383)..(1383): n is a, c, g, or t
  misc_feature (1392)..(1392): n is a, c, g, or t
  misc_feature (1395)..(1395): n is a, c, g, or t
  misc_feature (1404)..(1404): n is a, c, g, or t
  misc_feature (1410)..(1410): n is a, c, g, or t
  misc_feature (1413)..(1413): n is a, c, g, or t
  misc_feature (1428)..(1428): n is a, c, g, or t
atgggnytnacngtncaraarathaaytgggarcargtnaargartgggaymgnaartayytnatgmgnacnmgnwsnacncaraaygartaycarccngtnccnathgarwsnacngarggngaytayytnathatgccnggnggnacnmgnytnytngayttyttyaaycarytntaytgygtnaayathggncaraaraaycaraargtnaaygcngcnathaargargcnytngaymgntayggnttygtntgggaygcntaygcnacngaytayaargcnaargcngcnaarathathathgargayathytnggngaygargaytggccnggnaargtnmgnttygtnwsnacnggnwsngargcngtngaracngcnytnaayathgcnmgnytntayacnaaymgnccnytngtngtnacnmgngarcaygaytaycayggntggacnggnggngcngcngcngtnacnmgnytnmgnwsnttycarwsnggnytngcnggngaraaywsnggnwsnttywsngcncarathccnggnwsnwsntayaayaaygcngtnytnatggcnccnwsnccnaaygcnttycargaywsnaayggnaaytgyytnaargaygaraayggngarytnytnwsngtnaartayacnmgnmgnatgathgaraaytayggnccngarcargtngcngcngtnathacngargtnccncarggngtnggnwsnacnatgccnccntaygartayathccncarathmgnaaratgacnaargarytnggngtnytntggathaaygaygargtnytnacnggnttyggnmgnacnggnaartggttyggntaycarcaytayggngtncarccngayathathacnatgggnaarggnytnwsnwsnwsnwsnytnccngcnggngcngtngtngtnwsnaargarathgcngcnttyatggayaarcaymgntgggaracnggnwsnacntaygcnggncayccngtngcnatggcngcngtntgygcnaayytngargtnatgatggargaraayytngtngarcargcnaaraaywsnggngartayathmgnwsnaarytngarytnytncargaraarcayaarwsnathggnaayttygayggntgyggnytnytntggytngtngarathgtnaaygcngaracnaaracnccntaygtnaarytngaymgnaayttyacnmgnggnatgaayytnaaycarathccnacncarathathatggaraargcnytngaraarggngtnytnathggnggngtnatgccnaayacnatgmgnathggngcnwsnytnaaygtnwsnmgnggngayathgayaargcnatggaygcnytngaytaygcnytngaytayytngarwsnggngartggcarcarwsncaycaycaycaycaycay

SEQ ID NO: 17 - DNA, 1449 bp, Artificial Sequence
  Mutation of wild-type sequence.
  CDS (1)..(1449)
atgggcctgaccgtgcagaaaattaattgggaacaggtaaaagaatgggatcggaaatacctgatgcgcacacggtcaacccagaacgaatatcaaccggtcccgatcgaatcgaccgagggcgattacctgattatgccaggagggactcgcctcctggatttctttaaccagctgtattgcgtcaacatcggtcagaagaatcagaaggtgaatgcagcaattaaggaggcgttggaccgctatggctttgtgtgggatgcttatgcgacggactataaagccaaagcagcgaaaatcatcatcgaagatattctgggtgatgaagattggcccggtaaagtgcgtttcgtttcgactggttctgaagccgtagaaaccgcgctgaatatcgcccgtctttacacgaatcgccccttagtggttacacgcgaacatgactatcacggctggacgggaggtgctgctgccgtgacgcgcttacgcagctttcagagcggccttgccggcgaaaactcaggttcctttagtgcgcagattccagggtctagctacaataatgcggttctgatggcgccaagccctaatgcctttcaagatagcaacggcaactgcctgaaagacgagaacggtgaactgttaagcgtgaaatatacgcgtcgtatgatcgaaaactatgggcctgagcaagtagcggcagtcatcactgaagtaccgcaaggtgtcggctcaacaatgccgccgtacgaatatatcccgcaaattcgcaaaatgaccaaagaattaggcgtgttgtggatcaacgacgaagtcctgaccggatttggccgcaccggtaaatggttcggctatcagcactatggggttcagccagatatcattaccatgggtaaaggtctgtctagttcgtcactgcctgctggtgcagttgtggtgtccaaagagattgcggcattcatggacaaacatcgttgggagacaggctctacgtatgccggccatccggttgcgatggctgccgtttgtgcgaatctcgaagtaatgatggaagaaaatctggtggaacaagcgaagaacagtggggaatacattcgctccaaactggaattgctgcaagagaaacacaagtcgattggcaatttcgatggctgtggcttgctctggttggttgagattgtgaatgccgaaaccaaaactccgtacgtgaaactggaccgtaactttacccgtggtatgaacttaaaccagattccgacccagattatcatggagaaagcccttgagaaaggggttctgattggaggtgtcatgccgaacacgatgcgcattggagcatccctgaacgtcagccgtggcgatattgacaaggcaatggatgcgctcgattacgctctggattaccttgagagcggtgaatggcagcagagtcatcatcaccatcatcactaa

SEQ ID NO: 18 - protein, 482 aa, Artificial Sequence
  Synthetic Construct
MGLTVQKINWEQVKEWDRKYLMRTRSTQNEYQPVPIESTEGDYLIMPGGTRLLDFFNQLYCVNIGQKNQKVNAAIKEALDRYGFVWDAYATDYKAKAAKIIIEDILGDEDWPGKVRFVSTGSEAVETALNIARLYTNRPLVVTREHDYHGWTGGAAAVTRLRSFQSGLAGENSGSFSAQIPGSSYNNAVLMAPSPNAFQDSNGNCLKDENGELLSVKYTRRMIENYGPEQVAAVITEVPQGVGSTMPPYEYIPQIRKMTKELGVLWINDEVLTGFGRTGKWFGYQHYGVQPDIITMGKGLSSSSLPAGAVVVSKEIAAFMDKHRWETGSTYAGHPVAMAAVCANLEVMMEENLVEQAKNSGEYIRSKLELLQEKHKSIGNFDGCGLLWLVEIVNAETKTPYVKLDRNFTRGMNLNQIPTQIIMEKALEKGVLIGGVMPNTMRIGASLNVSRGDIDKAMDALDYALDYLESGEWQQSHHHHHH

SEQ ID NO: 19 - DNA, 1107 bp, Rhodosporidium toruloides
  CDS (1)..(1107)
  ref <308>: DATABASE ACCESSION NUMBER: GenBank / U60066.1
  ref <309>: DATABASE ENTRY DATE: 2001-10-16
  ref <313>: RELEVANT RESIDUES IN SEQ ID NO: (1)..(1107)
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgaattggacgcctttcatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgatggactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtag

SEQ ID NO: 20 - protein, 368 aa, Rhodosporidium toruloides
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 21 - DNA, 1107 bp, Artificial Sequence
  DAO1 from Rhodosporidium toruloides comprising mutations in the codons identified by the nucleotides at positions 160-162 and the codons identified by the nucleotides at positions 172-174 and the codons identified by the nucleotides at positions 637-639
  CDS (1)..(1107)
  misc_feature (639)..(639): n is a, c, g, or t
atgcactcgcagaagcgcgtcgttgtcctcggatcaggcgttatcggtctgagcagcgccctcatcctcgctcggaagggctacagcgtgcatattctcgcgcgcgacttgccggaggacgtctcgagccagactttcgcttcaccatgggctggcgcgtgytggacgcctcayatgacgcttacagacggtcctcgacaagcaaaatgggaagaatcgactttcaagaagtgggtcgagttggtcccgacgggccatgccatgtggctcaaggggacgaggcggttcgcgcagaacgaagacggcttgctcgggcactggtacaaggacatcacgccaaattaccgccccctcccatcttccgaatgtccacctggcgctatcggcgtaacctacgacaccctctccgtccacgcaccaaagtactgccagtaccttgcaagagagctgcagaagctcggcgcgacgtttgagagacggaccgttacgtcgcttgagcaggcgttcgacggtgcggatttggtggtcaacgctacgggacttggcgccaagtcgattgcgggcatcgacgaccaagccgccgagccaatccgcgggcaaaccgtcctcgtcaagtccccatgcaagcgatgcacgwsngactcgtccgaccccgcttctcccgcctacatcattccccgaccaggtggcgaagtcatctgcggcgggacgtacggcgtgggagactgggacttgtctgtcaacccagagacggtccagcggatcctcaagcactgcttgcgcctcgacccgaccatctcgagcgacggaacgatcgaaggcatcgaggtcctccgccacaacgtcggcttgcgacctgcacgacgaggcggaccccgcgttgaggcagaacggatcgtcctgcctctcgaccggacaaagtcgcccctctcgctcggcaggggcagcgcacgagcggcgaaggagaaggaggtcacgcttgtgcatgcgtatggcttctcgagtgcgggataccagcagagttggggcgcggcggaggatgtcgcgcagctcgtcgacgaggcgttccagcggtaccacggcgcggcgcgggagtcgaagttgtag

SEQ ID NO: 22 - protein, 368 aa, Artificial Sequence
  Synthetic Construct
MHSQKRVVVLGSGVIGLSSALILARKGYSVHILARDLPEDVSSQTFASPWAGACWTPHMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTSDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

SEQ ID NO: 23 - DNA, 1467 bp, Listeria seeligeri
  CDS (1)..(1467)
  ref <308>: DATABASE ACCESSION NUMBER: GenBank / NC_013891.1
  ref <309>: DATABASE ENTRY DATE: 2017-02-26
  ref <313>: RELEVANT RESIDUES IN SEQ ID NO: (2760202)..(2761668)
atgaccgatagaagaaacttaacgacgaatcaaggagtgccgattggggacaaccaaaattcgatgacagcgggattaaaaggaccaactttgttagaagattatgtgttaattgagaaattggcgcattttgatagagaacgtgttccagaacgtgtggtgcatgctcgtggtgctggtgcgcacggcaaatttgtaacgaaaaaaagcatgaaaaaatatacgaaagcacaatttttacaagaagaaggaacagagacagaggtttttgcgcgtttttctacagtaatccatggtcaacattcaccagaaacgcttcgtgatccacgtggtttttcggttaaattttatacggaagaaggtaactatgactttgtcggtaacaacttacctgtattctttatccgtgatgctatcaagtttcctgacgtaattcactccttgaagccagatccacgtactaacattcaagatggcaatcgttactgggacttctttagtttaactccggaagcgacgacgatgattacttatttatttagcgatgaggggactccggcatcttaccgcgaaattcgtggttcaagcgtacatgcctttaaatggataaacgaagaaggtaagactgtttatgtaaaactacgctgggttccaaaagcaggaatcgtcaatctttcaactgatcaagcagcacaaattcaagcaaaagaatttaaccatgctagtcgcgatttgtatgaagcaattgagaatggtgattatccagagtgggatttatatgtgcaagtgctagatccaaaagacttggataattacgacttcaatccgcttgatgcaaccaaagactggtttgaagatgtatttccatacgagcatgttggaacaatgacattaaatcgtaatccggataatatttttgctgaaacagaatcagttggctttaatccaggtgtgcttgtgccggggatgttaccttctgaggaccgtttactacaggggcgattgttctcttactctgatacgcaaagacaccgcgttggacctaactacttacaattaccaatcaacagcccaaaaactcctgttgataacaaccaacgtgatggacagatgccgtttaaacagcaaacaagttcgattaattatgaaccaaatagttatgatacagaaccaaaagaaaaccctgcatatatcgagcctgagcaagaaattcgtggggatatctctggccgactagtggcagaaaagccaaataactttggtcatgctaaagaagtttggaagcgttactcagatgcagaacgtgcggctcttgtgaaaaatattgtagacgattgggaaggtgtgcgcgaagatattaagattcgcaacttgcgcaatttctatcaagtagagccggaatttgcagaacgtgtggctgctggaactggaattaaccttgctgaacatgtgatagatttaaaataa

SEQ ID NO: 24 - protein, 488 aa, Listeria seeligeri
MTDRRNLTTNQGVPIGDNQNSMTAGLKGPTLLEDYVLIEKLAHFDRERVPERVVHARGAGAHGKFVTKKSMKKYTKAQFLQEEGTETEVFARFSTVIHGQHSPETLRDPRGFSVKFYTEEGNYDFVGNNLPVFFIRDAIKFPDVIHSLKPDPRTNIQDGNRYWDFFSLTPEATTMITYLFSDEGTPASYREIRGSSVHAFKWINEEGKTVYVKLRWVPKAGIVNLSTDQAAQIQAKEFNHASRDLYEAIENGDYPEWDLYVQVLDPKDLDNYDFNPLDATKDWFEDVFPYEHVGTMTLNRNPDNIFAETESVGFNPGVLVPGMLPSEDRLLQGRLFSYSDTQRHRVGPNYLQLPINSPKTPVDNNQRDGQMPFKQQTSSINYEPNSYDTEPKENPAYIEPEQEIRGDISGRLVAEKPNNFGHAKEVWKRYSDAERAALVKNIVDDWEGVREDIKIRNLRNFYQVEPEFAERVAAGTGINLAEHVIDLK

SEQ ID NO: 25 - DNA, 1464 bp, Artificial Sequence
  codon adapted sequence encoding protein having catalase activity
atgaccgatcgccgcaacctgaccaccaaccagggcgtgccgattggcgataaccagaacagcatgaccgcgggcctgaaaggcccgaccctgctggaagattatgtgctgattgaaaaactggcgcattttgatcgcgaacgcgtgccggaacgcgtggtgcatgcgcgcggcgcgggcgcgcatggcaaatttgtgaccaaaaaaagcatgaaaaaatataccaaagcgcagtttctgcaggaagaaggcaccgaaaccgaagtgtttgcgcgctttagcaccgtgattcatggccagcatagcccggaaaccctgcgcgatccgcgcggctttagcgtgaaattttataccgaagaaggcaactatgattttgtgggcaacaacctgccggtgttttttattcgcgatgcgattaaatttccggatgtgattcatagcctgaaaccggatccgcgcaccaacattcaggatggcaaccgctattgggatttttttagcctgaccccggaagcgaccaccatgattacctatctgtttagcgatgaaggcaccccggcgagctatcgcgaaattcgcggcagcagcgtgcatgcgtttaaatggattaacgaagaaggcaaaaccgtgtatgtgaaactgcgctgggtgccgaaagcgggcattgtgaacctgagcaccgatcaggcggcgcagattcaggcgaaagaatttaaccatgcgagccgcgatctgtatgaagcgattgaaaacggcgattatccggaatgggatctgtatgtgcaggtgctggatccgaaagatctggataactatgattttaacccgctggatgcgaccaaagattggtttgaagatgtgtttccgtatgaacatgtgggcaccatgaccctgaaccgcaacccggataacatttttgcggaaaccgaaagcgtgggctttaacccgggcgtgctggtgccgggcatgctgccgagcgaagatcgcctgctgcagggccgcctgtttagctatagcgatacccagcgccatcgcgtgggcccgaactatctgcagctgccgattaacagcccgaaaaccccggtggataacaaccagcgcgatggccagatgccgtttaaacagcagaccagcagcattaactatgaaccgaacagctatgataccgaaccgaaagaaaacccggcgtatattgaaccggaacaggaaattcgcggcgatattagcggccgcctggtggcggaaaaaccgaacaactttggccatgcgaaagaagtgtggaaacgctatagcgatgcggaacgcgcggcgctggtgaaaaacattgtggatgattgggaaggcgtgcgcgaagatattaaaattcgcaacctgcgcaacttttatcaggtggaaccggaatttgcggaacgcgtggcggcgggcaccggcattaacctggcggaacatgtgattgatctgaaa

SEQ ID NO: 26 - DNA, 21 bp, Escherichia coli
aattgtgagcggataacaatt

SEQ ID NO: 27 - DNA, 31 bp, Artificial Sequence
  Synthetic promoter comprising sequnces from E. coli trp-promoter and E. coli lacUV5 promoter
tgttgacaattaatcatccggctcgtataat

SEQ ID NO: 28 - DNA, 70 bp, Artificial Sequence
  Rho-independent terminator
cgccgctgagaaaaagcgaagcggcactgctctttaacaatttatcagacaatctgtgtgggcactcgac

SEQ ID NO: 29 - DNA, 27 bp, Artificial Sequence
  Mini-cistron
atgtatcgattaaataaggaggaataa

SEQ ID NO: 30 - DNA, 158 bp, Artificial Sequence
  Transcription termination signal
tgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctt